(19) United States
(12) Patent Application Publication
(10) Pub. No.: US 2026/0021297 A1
(43) Pub. Date: Jan. 22, 2026

(54) SYSTEMS AND METHODS RELATED TO NEUROMODULATION OF GASTROINTESTINAL DYSMOTILITY

(71) Applicant: Duke University, Durham, NC (US)

(72) Inventors: Warren Grill, Durham, NC (US); Bradley Barth, Durham, NC (US)

(21) Appl. No.: 19/275,099

(22) Filed: Jul. 21, 2025

Related U.S. Application Data
(60) Provisional application No. 63/674,118, filed on Jul. 22, 2024.

(51) Int. Cl. A61N 1/36

(52) U.S. Cl. CPC A61N 1/36007; A61N 1/36178

(57) ABSTRACT

The present disclosure provides systems and methods relating to the neuromodulation of gastrointestinal dysmotility. In particular, the present disclosure provides systems and methods for delivering temporal patterns of electrical stimulation comprising burst-patterned stimulation according to various stimulation parameters to treat gastrointestinal dysmotility disorders in a subject. The system may be implantable.

### CROSS REFERENCE TO RELATED APPLICATIONS

This application claims priority to and the benefit of U.S. Provisional Patent Application Ser. No. 63/674,118 filed Jul. 22, 2024, which is incorporated herein by reference in its entirety and for all purposes.

### STATEMENT REGARDING FEDERALLY SPONSORED RESEARCH OR DEVELOPMENT

This invention was made with Government support under Federal Grant No. R01 DK119795 awarded by the National Institutes of Health National Institute of Diabetes & Digestive & Kidney Diseases (NIH/NIDDK). The Federal Government has certain rights to the invention.

### FIELD

The present disclosure provides systems and methods relating to the neuromodulation of gastrointestinal dysmotility. In particular, the present disclosure provides systems and methods for delivering temporal patterns of electrical stimulation comprising burst-patterned stimulation according to various stimulation parameters to treat gastrointestinal dysmotility disorders in a subject.

### BACKGROUND

Electrical stimulation of peripheral nerves is one approach to restore communication and control of gastrointestinal organs and can provide relief from various gastrointestinal dysmotility disorders, such as gastroparesis, fecal incontinence, and inflammatory bowel disease. Activity in the sacral nerves can both increase and decrease motility, but the same stimulation parameters are used to treat constipation and fecal incontinence, conditions with overlapping and opposite motility symptoms. Further, although electrical stimulation evokes propagating contractions, continuous or tonic stimulation of the sacral nerve has failed to treat slow-transit constipation more effectively than sham stimulation in a randomized, double-blind, placebo-controlled, crossover study. Therefore, there is a need to develop alternatives to continuous, tonic stimulation used in clinical sacral nerve stimulation for the treatment of gastrointestinal dysmotility.

### SUMMARY

Embodiments of the present disclosure include methods of treating a gastrointestinal dysmotility disorder in a subject in need thereof. In accordance with these embodiments, the method includes applying a temporal pattern of electrical stimulation comprising burst-patterned stimulation to a target nerve or a set of target nerves in a subject having at least one symptom of a gastrointestinal dysmotility disorder, such that the application of the temporal pattern of electrical stimulation modulates gastrointestinal motility in the subject.

In some embodiments, the temporal pattern of electrical stimulation is applied according to one or more stimulation parameters comprising stimulation pulse amplitude, stimulation pulse width, stimulation pulse frequency, stimulation pulse waveform shape, and/or ramp time.

In some embodiments, the burst-patterned stimulation is applied according to one or more stimulation parameters comprising pulse amplitude, pulse width, pulse shape, pulse repetition frequency, burst duration, interburst interval, and ramp time.

In some embodiments, the burst-patterned stimulation comprises at least two identical bursts. In some embodiments, the burst-patterned stimulation comprises at least two non-identical bursts.

In some embodiments, the pulse repetition frequency is from about 0.1 Hz to about 30 Hz. In some embodiments, the pulse repetition frequency is from about 8 Hz to about 25 Hz.

In some embodiments, the burst duration is from about 10 seconds to about 60 seconds. In some embodiments, the burst duration is from about 15 seconds to about 45 seconds.

In some embodiments, the interburst interval is from about 10 seconds to about 120 seconds. In some embodiments, the interburst interval is from about 40 seconds to about 90 seconds.

In some embodiments, the pulse shape comprises a rectangular shape, a sinusoidal shape, a ramp, an exponential rise, and/or an exponential fall. In some embodiments, the pulse shape is monophasic or biphasic.

In some embodiments, the gastrointestinal motility disorder comprises a hypermotility disorder. In some embodiments, the gastrointestinal motility disorder comprises a hypomotility disorder. In some embodiments, the gastrointestinal motility disorder comprises colonic dysmotility. In some embodiments, the gastrointestinal motility disorder comprises constipation.

In some embodiments, the target nerve or set of target nerves comprise an extrinsic nerve or set of extrinsic nerves, or intrinsic (enteric) nerves. In some embodiments, the extrinsic nerve or set of extrinsic nerves comprise vagal afferent or vagal efferent nerves, splanchnic nerves, pelvic nerves, rectal nerves, lumbar colonic nerves, hypogastric verves, and/or sacral nerves. In some embodiments, the intrinsic nerves comprise nerves that lie within the wall of the gastrointestinal tract. In some embodiments, the extrinsic nerve or set of extrinsic nerves, or the intrinsic (enteric) nerves innervate the gastrointestinal tract. In some embodiments, the target nerve or a set of target nerves comprises the sacral nerve, and wherein the gastrointestinal dysmotility disorder comprises constipation. In some embodiments, the target nerve or a set of target nerves comprises the sacral nerve, and wherein the gastrointestinal dysmotility disorder comprises disorders of gut-brain interactions.

In some embodiments, the at least one symptom of a gastrointestinal dysmotility disorder comprises early satiety, nausea, vomiting, bloating, diarrhea, constipation, involuntary weight loss, abdominal pain, abdominal swelling (distention), and/or intrarectal pressure.

In some embodiments, the subject is a human, and wherein application of the temporal pattern of electrical stimulation comprising burst-patterned stimulation treats the gastrointestinal dysmotility disorder in the subject.

In some embodiments, the temporal pattern of electrical stimulation comprising the burst-patterned stimulation comprises at least one additional temporal pattern of electrical stimulation. In some embodiments, the additional temporal pattern of electrical stimulation comprises a second burst-patterned stimulation.

Embodiments of the present disclosure also include a method of treating a gastrointestinal dysmotility disorder in a human subject in need thereof. In accordance with these embodiments, the method includes programming a pulse generator to output a temporal pattern of electrical stimulation comprising burst-patterned stimulation to a target nerve or set of target nerves in a subject having at least one symptom of a gastrointestinal dysmotility disorder, and delivering the temporal pattern of electrical stimulation to the subject, such that delivering the temporal pattern of electrical stimulation modulates motility in the subject and thereby treats the gastrointestinal dysmotility disorder.

In some embodiments, the at least one temporal pattern of electrical stimulation comprising the burst-patterned stimulation is delivered according to stimulation parameters determined to treat the gastrointestinal dysmotility disorder. In some embodiments, the burst-patterned stimulation is applied according to one or more stimulation parameters comprising pulse amplitude, pulse width, pulse shape, pulse repetition frequency, burst duration, interburst interval, and ramp time.

In some embodiments, the gastrointestinal motility disorder comprises a hypermotility disorder. In some embodiments, the gastrointestinal motility disorder comprises a hypomotility disorder. In some embodiments, the gastrointestinal motility disorder comprises colonic dysmotility. In some embodiments, the gastrointestinal motility disorder comprises constipation.

In some embodiments, the at least one symptom of a gastrointestinal dysmotility disorder comprises early satiety, nausea, vomiting, bloating, diarrhea, constipation, involuntary weight loss, abdominal pain, abdominal swelling (distention), and/or intrarectal pressure.

In some embodiments, the pulse generator is configured to output the temporal pattern of electrical stimulation comprising the burst-patterned stimulation and at least one additional temporal pattern of electrical stimulation. In some embodiments, the pulse generator is configured to allow the subject to alternate between delivering the temporal pattern of electrical stimulation comprising the burst-patterned stimulation and the at least one additional temporal pattern of electrical stimulation. In some embodiments, the additional temporal pattern of electrical stimulation comprises a second burst-patterned stimulation.

Embodiments of the present disclosure also include a method of selecting a temporal pattern of electrical stimulation to treat a gastrointestinal dysmotility disorder in a human subject in need thereof. In accordance with these embodiments, the method includes delivering a first burst-patterned stimulation to a target nerve or a set of target nerves in a subject having at least one symptom of a gastrointestinal dysmotility disorder and assessing efficacy of stimulation and/or a degree of relief of the at least one symptom; determining a second burst-patterned stimulation by adjusting a stimulation parameter of the first burst-patterned stimulation; delivering the second burst-patterned stimulation to the target nerve or the set of target nerves in the subject and reassessing the efficacy of stimulation and/or the degree of relief of the at least one symptom; and selecting for treatment one of the first burst-patterned stimulation or the second burst-patterned stimulation based on the efficacy of stimulation and/or the degree of relief.

In some embodiments, assessing and reassessing the efficacy of stimulation and/or the degree of relief involves measuring intrarectal pressure (e.g., using anorectal manometry).

In some embodiments, the stimulation parameter comprises one or more of pulse amplitude, pulse width, pulse shape, pulse repetition frequency, burst duration, interburst interval, and/or ramp time.

In some embodiments, the method further comprises adjusting a second stimulation parameter. In some embodiments, the method further comprises readjusting the adjusted stimulation parameter.

In some embodiments, the at least one symptom comprises early satiety, nausea, vomiting, bloating, diarrhea, constipation, involuntary weight loss, abdominal pain, abdominal swelling (distention), and/or intrarectal pressure. In some embodiments, assessment of the at least one symptom comprises measurement of intrarectal pressure. In some embodiments, intrarectal pressure is measured using anorectal manometry. In some embodiments, assessing the efficacy of stimulation and/or the degree of relief of the at least one symptom comprises determining a paired-burst response ratio corresponding to quantification of the first response to the first burst-patterned stimulation as compared to quantification of the second response of the second burst-patterned stimulation.

In some embodiments, the method selects a first treatment for at least one symptom of a first gastrointestinal dysmotility disorder, and the method is repeated to select at least a second treatment for at least one symptom of a second gastrointestinal disorder.

Embodiments of the present disclosure also include a system for treating gastrointestinal dysmotility disorder in a subject in need thereof. In accordance with these embodiments, the system includes a pulse generator that includes a processor; a lead electrically coupled to the device; and an electrode electrically coupled to the lead and positioned to transmit an electrical stimulation signal to a target nerve or set of target nerves in the subject. In some embodiments, the processor is configured to control the pulse generator to provide the electrical stimulation signal to the target nerve or the set of target nerves in the subject in a first temporal pattern comprising burst-patterned stimulation. In some embodiments, the application of the first temporal pattern modulates gastrointestinal motility in the subject, thereby treating the gastrointestinal dysmotility disorder.

In some embodiments, the pulse generator is implantable.

In some embodiments, the gastrointestinal motility disorder comprises constipation.

In some embodiments, the system further comprises a remote control device that is configured to control the implantable pulse generator and the electrical stimulation signal being provided to the target nerve or set of target nerves. In some embodiments, the remote control device is in wireless communication with the pulse generator.

In some embodiments, the processor is configured to adjust one or more stimulation parameters of the electrical stimulation signal in response to user input received at the remote control device.

In some embodiments, the pulse generator is cycled off in response to user input received at the remote control device.

In some embodiments, the system further includes a remote control device that is configured to control the pulse generator. In some embodiments, the remote control device is configured to receive a user input to select the first temporal pattern or the second temporal pattern for the electrical stimulation signal being provided to the target nerve or set of target nerves.

In some embodiments, the system further includes a programmer in communication with the pulse generator and configured to control the processor to modify one or more stimulation parameters of the electrical stimulation signal.

In some embodiments, the first temporal pattern includes an interburst interval between burst durations during which the burst-patterned stimulation is provided, and the interburst interval is not dependent on the length of a refractory period of the subject's colon.

In some embodiments, the interburst interval is from about 10 seconds to about 70 seconds. In some embodiments, the interburst interval is from about 30 seconds to about 50 seconds.

In some embodiments, the pulse repetition frequency during each burst duration is from about 1 Hz to about 30 Hz. In some embodiments, the pulse repetition frequency during each burst duration is from about 5 Hz to about 25 Hz. In some embodiments, the pulse repetition frequency during each burst duration is from about 10 Hz to about 20 Hz.

In some embodiments, the burst duration is from about 10 seconds to about 60 seconds. In some embodiments, the burst duration is from about 15 seconds to about 45 seconds.

### BRIEF DESCRIPTION OF THE DRAWINGS

FIG. 1: Representative schematic diagram illustrating burst-patterned stimulation. As with continuous stimulation, burst-patterned stimulation can be turned on and off, for example, using a real-time controller and/or by a patient or medical professional. Continuous stimulation involves the application of pulses at a cadence defined by the pulse repetition frequency, whereas burst stimulation involves the application of pulses at a cadence defined by both the pulse repetition frequency and the overarching burst frequency. In burst stimulation, pulses are delivered in bursts for a defined burst duration, separated by interburst intervals during which no pulses are applied. The combination of the burst duration and the interburst interval equals the inverse of the burst frequency. As shown in the schematic diagram, burst-patterned stimulation delivers a programmed waveform according to various stimulation parameters, including but not limited to, pulse amplitude, pulse width, pulse shape, pulse repetition frequency, burst duration, interburst interval, and ramp time. The pulses are repeated at a pulse repetition frequency for a period of time defined by the burst duration, which can include on ramp time and off ramp time; and, the interburst interval defines the period of time between bursts when no pulses are being applied. The pulse shape can include, but is not limited to, a rectangular shape, a sinusoidal shape, a ramp, an exponential rise, and/or an exponential fall. As shown in the schematic diagram, the neuromodulation systems of the present disclosure are configured to deliver therapeutically effective neuromodulation treatment on a patient-by-patient basis.

FIGS. 2A-2E: Representative drawings related to the use of burst pelvic nerve stimulation to evoke propagating contractions in the isolated mouse colon. Isolated colon preparation with smooth muscle calcium imaging and pelvic nerve stimulation is shown in FIG. 2A. FIGS. 2Bi and 2Bii show spatiotemporal maps of calcium waves evoked by fluid distension (FIG. 2Bi), and pelvic nerve stimulation (FIG. 2Bii). The rate of calcium waves per minute before, during, and after pelvic nerve stimulation delivered tonically (gray triangle) and in a 20 Hz, 5 s burst pattern with 60 s between bursts (red circles) are shown in FIG. 2C. Each symbol represents unique mouse colons (n=11, t ratio=2.9, p<0.01). Representative calcium waves evoked by (FIG. 2D) tonic and (FIG. 2E) burst-patterned pelvic nerve stimulation are shown in FIGS. 2D and 2E. Colorimetric scale is consistent FIGS. 2B-2D. The vertical and horizontal scale bars in FIGS. 2Bi and 2Bii are consistent, as are scale bars in FIGS. 2C and 2D.

FIGS. 3A-3I: Representative data related to the evaluation of parameters of burst-patterned stimulation in a computational model. A schematic of the computational model of colonic motility including a biophysical cellular network (blue) and computational fluid dynamics (red) is shown in FIG. 3A. Simulated pellet propagation in the virtual colon superimposed on a spatiotemporal map of the colon diameter is shown in FIG. 3B. The average velocity of fecal pellets in the computational model and the isolated mouse colon is shown in FIG. 3C. Individual points in the first two rows are distinct random instances of the model and unique mouse colons (see, e.g., M. Costa et al., Motor patterns in the proximal and distal mouse colon which underlie formation and propulsion of feces. Neurogastroenterology and motility: The official journal of the European Gastrointestinal Motility Society 33, c14098 (2021)). Mean and standard error of the mean are shown in black bars. Simulated colonic emptying of four pellets in a model is shown in FIGS. 3D-3G. FIG. 3D shows normal transit, FIG. 3E shows slow transit, FIG. 3F shows slow transit with burst-patterned stimulation, and FIG. 3G shows slow transit with tonic stimulation. Lines and shaded regions indicate the mean position and standard error of the mean position across 4-10 random instances of the model. The mean pellet velocity from 10 random instances of the model for normal transit, slow transit, slow transit with burst-patterned stimulation, and slow transit with tonic stimulation is shown in FIG. 3H. The fecal output in pellets per minute from 10 random instances of the model for normal transit, slow transit, slow transit with burst-patterned stimulation, and slow transit with tonic stimulation is shown in FIG. 3I.

FIGS. 4A-4F: Representative data related to the response of the model to various burst stimulation parameters. For example, the response rate across 10 random instances of the model for varying combinations of burst pattern parameters is shown in FIG. 4A. Four candidate patterns (a, b, c & d) increased fecal output by at least 50% in at least 7 of 10 instances of the model. Each circle represents one pattern of stimulation, and the size and color of the circle indicates the percentage of trials in which fecal output increased by at least 50%. FIG. 4B is an illustration of anesthetized rat with lumbosacral nerve stimulation and anorectal manometry. FIG. 4C is a schematic of the lumbosacral plexus and the evoked pressure responses from up to 5 rats. Scale bars, 5 mmHg and 10 s, are consistent across the traces shown in FIG. 4C. Each trace is a representative recording from one rat, with line colors corresponding to different rats. Colonic pressures evoked by candidate patterns of sacral nerve stimulation are shown in FIGS. 4D-4F. The figures are annotated with the following burst stimulation parameters: pulse repetition frequency, burst duration, and interburst interval (where 2 Hz 20/60 s refers to pattern with 2 Hz bursts lasting 20 s in duration with 60 s between bursts). FIG. 4D shows anorectal pressures in urethane-anesthetized rats comparing candidate patterns of the burst type sacral nerve stimulation identified in the model to conventional, 14 Hz tonic sacral nerve stimulation. Lines and shaded regions indicate mean pressures and standard errors of the mean pressure in n=5 rats. FIG. 4E shows the magnitude of contractions during stimulation expressed as the full width at half amplitude pressure-time integral (mmHg s) for patterns of sacral nerve stimulation (n=5, ANOVA F ratio=8.6, p<0.001) (FIG. 4E). FIG. 4F shows the mean pressure of contractions during different patterns of the burst type sacral nerve stimulation (n=5, ANOVA F ratio=8.4, p<0.001).

FIGS. 5A-5F: Representative data related to the use of burst stimulation to increase fecal output of awake, behaving rats with slow-transit constipation. FIG. 5A shows both a schematic and an x-ray of bipolar electrode nerve cuff surgically placed on L6-S1 nerve trunk and tunneled subcutaneously to percutaneous headcap. FIG. 5B is an illustration of post-operative rat with electrical stimulation delivered by tethered connection. The drawings are representative recordings of fecal mass over four nights. In FIG. 5C, fecal events are denoted by blue triangles, and the sum of fecal mass between 3:00 pm and 9:00 am the following day are reported for each night. FIG. 5D shows the percent change from baseline fecal output (night 1) and constipated fecal output (night 2) in control and slow-transit constipation (STC) experimental groups. n=7, t ratio=−10.8, p<0.0001. FIG. 5E shows the fecal output measured over four consecutive nights in seven rats. Each dot represents one rat, and each rat underwent each condition: negative control (gray circle), loperamide control (light blue diamond), burst sacral nerve stimulation (dark blue square), and tonic sacral nerve stimulation (red triangle). Loperamide was delivered to STC groups (light blue, dark blue, red) at noon preceding nights 2, 3, and 4. Sacral nerve stimulation was delivered overnight 3 from noon to noon in sacral nerve stimulation groups (dark blue, red). FIG. 5F shows the percent change in fecal output from constipated state (night 2) to constipated with stimulation (night 3). FIG. 5F shows the same groups as described in FIG. 5E (n=7, ANOVA F ratio=5.6, p<0.005).

FIGS. 6A-6F: Representative data related to the sensitivity to parameters of burst-patterned stimulation in anesthetized rat. FIG. 6A shows the representative anorectal pressures evoked by 5 s bursts with varying pulse repetition frequencies. FIG. 6B shows normalized area-under-the-curve of anorectal pressure response to 5 s burst of sacral nerve stimulation with increasing pulse repetition frequency. Representative anorectal pressures evoked by 20 Hz bursts with varying burst durations are shown in FIG. 6C. Normalized area-under-the-curve of anorectal pressure response to 20 Hz burst of sacral nerve stimulation with increasing burst duration are shown in FIG. 6D. Representative pressures evoked by one pair of bursts delivered at 20 Hz for 40 s each with varying interburst intervals are shown in FIG. 6E. The paired-burst ratio, quantified as the area-under-the-curve of the anorectal pressure response of the second burst normalized to the response of the first burst, with increasing interburst interval is shown in FIG. 6F. The response variables within rats are normalized to the asymptote, and the summary data are fit with a three-parameter sigmoid function across all data normalized within rats. The mean and standard error of the mean are shown in black. Lines and shaded regions indicate the sigmoid fit and the standard error of the fit. n=4-7 rats.

FIGS. 7A-7E: Representative data related to the minimum threshold to evoke CMCs varied with pulses per burst and frequency of pelvic nerve stimulation. FIG. 7A is a schematic of the isolated colon preparation with pelvic nerve stimulation and myoelectric recording. FIG. 7B is a representative myoclectric recording of a colonic motor complex evoked by pelvic nerve stimulation. FIGS. 7C-7E show the threshold current (mA) to evoke a CMC with varying stimulation parameters. FIG. 7C shows the threshold current while holding constant frequency. FIG. 7D shows the threshold current while varying frequency and holding burst duration constant. FIG. 7E shows the threshold current while holding pulses per burst constant while varying burst duration. As shown in FIGS. 7D and 7E, the threshold to evoke CMCs for varying burst duration and frequency was not normally distributed (n=10, W=0.93, p-value=0.001).

FIG. 8: Representative analysis of the distribution of neuron positions in the computational model by population along the length of colon.

FIGS. 9A-9H: Representative data regarding the components of the integrated computational model of colonic motility. Connectivity in the computational network model. FIG. 9A is a graphical representation of the network model with neurons (circles), smooth muscle fibers (squares), and interstitial cells of Cajal (triangles). Connections include intrinsic neuron projections (blue), gap junctions (red), and pelvic innervation (gold). Lines terminating in arrowheads and dots represent excitatory and inhibitory connections, respectively. The modeling of the colonic motility may use the immersed boundary method, as shown in FIG. 9B. The colon and a 2-mm pellet are modeled in the Lagrangian field (Γ) in the fixed Cartesian grid of the Eulerian domain (Ω), as can be seen in FIG. 9B. The Lagrangian points of colon are connected to adjacent points on the same boundary by Hookcan springs (k_{SC}), torsional springs (k_{BC}, ϕ°), and longitudinal muscle fibers (k_{L}), as shown in FIG. 9C. The Lagrangian points of the colon on opposite walls are connected by circular muscle fibers (k_{C}). The Lagrangian points of the pellet are connected to adjacent points by Hookcan springs (k_{SP}) and torsional springs (k_{BP}, θ°). FIG. 9D is a graph showing the active force coefficient as a function of intracellular Ca²⁺. The negative force was used to model smooth muscle active relaxation. As used in the model, active (blue), passive (gray), and total tension (red) are shown in FIG. 9E as a function of the length of muscle fibers to replicate the tension-length relationship. Calcium transients from slowly adapting and rapidly adapting myenteric neurons were compared to the membrane potential of simulated mechanosensitive neurons in FIGS. 9F and 9G, respectively. The mucosal reflex was modeled as serotonin release from artificial point cells with varying firing rate based on the circumferential strain in the colon and whether a pellet was brushing against a given cell. In the graph of firing rate v. strain shown in FIG. 9H, each point represents a randomly generated firing rate with 25% noise when brushed (gray) and unbrushed (blue). In FIG. 9H, the expected values for brushed (gray) and unbrushed (black) conditions are plotted as solid lines as a function of strain. The drawings use the following abbreviations: IPAN, intrinsic primary afferent neuron; AC, ascending cholinergic interneuron; DN, descending nitrergic interneuron; DS, descending somatostatinergic interneuron; CI, circular inhibitory motor neuron; LI, longitudinal inhibitory motor neuron; CE, circular excitatory motor neuron; LE, longitudinal excitatory motor neuron; CM, circular muscle fiber; LM, longitudinal muscle fiber; ICC, interstitial cell of Cajal; MY, myenteric ICC; SMP, submucosal plexus ICC.

FIG. 10: Representation of a five-state Markovian kinetic gating model of Nav1.5. The channel started in the closed state I) and was activated by voltage entering the closed activated state (CA). The channel could proceed to inactivation (IA) or open (O). The channel inactivated (IO) and could recover from the inactive state without opening (IO→IA→CA). (Adapted from A. Beyder et al., Mechanosensitivity of Nav1.5, a voltage-sensitive sodium channel. 588, 4969-4985 (2010).)

FIGS. 11A-11I: Representative data pertaining to the process of calibrating biophysically realistic model of the Interstitial cells of Cajal (ICC).parameters of a mechanosensitive Nav1.5 kinetic model are determined. Performance cost for 10 random seeds of particle swarm optimization is shown as a function of iteration for pressure-independent calibration in FIG. 11A, and as a function of pressure-sensitive calibration in FIG. 11D. Each line in FIGS. 11A, 11D, and 11G represents the best performance within each independent swarm. Distribution of parameter values (normalized within their minimal and maximal values) for the parameters in the pressure-independent calibration is shown in FIG. 11B, and for the parameters in the pressure-sensitive calibration is shown in FIG. 11E. The distributions shown in FIGS. 11B, 11E, and 11H are drawn from 10 independent swarms. Voltage-dependent deactivation and activation of the peak Nav1.5 current is shown in FIGS. 11C and 11F, respectively. The model includes calibrating the parameters of the ICC model to replicate the Lees-Green model. Performance cost for 10 random seeds of particle swarm optimization as a function of iteration for ICC model calibration is shown in FIG. 11G. The distribution of calibrated parameter values I shown in FIG. 11H. Intracellular Ca²⁺ and transmembrane potential (Vₘ) from the calibrated model of ICC (solid, black traces) and the target responses from the Lees-Green model (thin, gray traces) is shown in FIG. 11I.

FIGS. 12A-12L: Representative data of mechanosensitive mechanisms and the optimization of model parameters. Data and information relating to mechanosensation in neurons is shown in FIGS. 12A to 12G. Performance cost for 10 random seeds of particle swarm optimization as a function of iteration for rapidly-adapting AH-type neurons, rapidly-adapting S-type neurons, and slowly-adapting S-type neurons is shown in FIGS. 12A, 12B and 12C, respectively. Each line represents the best performance within each independent swarm. Transmembrane potential (Vm) of the best-performing parameters in (a-c) in response to 20% deformation over time is shown in FIG. 12D. Distribution of optimal parameter values from 10 independent swarms (normalized within their minimal and maximal values) for the optimized parameters in rapidly-adapting AH-type neurons, rapidly-adapting S-type neurons, and slowly-adapting S-type neurons is shown in FIGS. 12E, 12F and 12G, respectively. Data and information relating to mechanosensation in ECC is shown in FIGS. 12H to 12J. ECC firing rate in response to pulsed deformation is shown in FIG. 12H. ECC firing rate in response to ramped deformation is shown in FIG. 12I. Firing rate for brushed (gray) and unbrushed (black) mucosa as a function of strain in FIG. 12J. The point cloud for each trace represents random samples of the firing rate for a given strain and brushed state. Data and information relating to mechanosensation in SMF is shown in FIGS. 12K and 12L. Transmembrane potential and stretch-dependent potassium current in SMF in response to tension is shown in FIG. 12K. Peak stretch-dependent potassium current in SMF as a function of applied tension is shown in FIG. 12L. In the drawings, transmembrane potential (voltage) is abbreviated as Vm.

FIGS. 13A-13Z: Representative data related to biophysical mechanisms underlying network connections. FIG. 13A shows nicotinic cholinergic receptor Excitatory Post Synaptic potential (EPSP). Muscarinic cholinergic receptor Excitatory Post Synaptic Current (EPSC) is shown in FIG. 13B. Muscarinic cholinergic receptor EPSC dose response is shown in FIG. 13C. P2_{X2} EPSP is shown in FIG. 13D. Cholecystokinin mediated EPSP is shown in FIG. 13E. Summed 5-HT3 and 5-HT_{1P} EPSP is shown in FIG. 13F. Somatostatin-mediated inhibitory post synaptic potential (IPSP) amidst depolarizing pulses at 600 nA every 9 s and 25 nA every 2 s are shown in FIGS. 13G and 13H, respectively. CGRP-evoked postsynaptic depolarization and spiking is shown in FIG. 13I. Substance P EPSP is shown in FIG. 13J. Vasoactive intestinal peptide EPSP is shown in FIG. 13K. Enkephalin-mediated postsynaptic inhibition amidst 5 Hz nicotinic EPSPs, intrinsic spiking, and mean firing rate normalized to baseline spiking are shown in FIGS. 13L, 13M and 13N, respectively. The percent inhibition from norepinephrine demonstrating time constants, the inhibition of 5 Hz nicotinic EPSPs, and the dose response curve of inhibition mediated by norepinephrine are shown in FIGS. 13O, 13P and 13Q, respectively. Inhibitory junction potentials in smooth muscle by 1 Hz are shown in FIGS. 13R, 13T, and 13V. Inhibitory junction potentials in smooth muscle by 5 Hz are shown in FIGS. 13S, 13U, and 13W. Inhibitory junction potentials with only purinergic mechanisms are shown in FIGS. 13R and 13S., Inhibitory junction potentials with only nitrergic mechanisms are shown in FIGS. 13T and 13U. Inhibitory junction potentials with both purinergic and nitrergic mechanisms are shown in FIGS. 13V and 13W. Excitatory junction potentials in smooth muscle evoke off-responses (black) in transmembrane potential as shown in FIG. 13X and evoke contractile force as shown in FIG. 13Y. The contractile force is occluded by atropine (gray) as shown in FIG. 13Z. The following abbreviations are used in the drawings: Carbachol (CCh); Encephalin (Enk); Norepinephrine (NE); Electric Field Stimulation (EFS).

FIGS. 14A and 14B: Representative data related to potassium conductance reduction by G-protein coupled receptors. Metabotropic reaction kinetics that contribute to reduced potassium conductance are shown in FIG. 14A. Normalized maximum potassium conductance as a function of GR concentration is shown in FIG. 14B. The following abbreviations are used in the drawings: R, receptor; pG, G-protein precursor; GR, G-protein coupled receptor complex. K1s, K1c, & K1v refer to forward reaction rates for substance P, CGRP, and VIP, respectively, and K2s, K2c, & K2v refer to the respective reverse reaction rates. K3 and K4 are the forward and reverse reaction rates producing the G-protein coupled receptor complex.

FIGS. 15A-15B: Representative data related to the fluid distension external forcing function. FIG. 15A shows the maximum amplitude of the forcing function against time. The time has been offset such that t=0 indicates when the applied force reaches the proximal end of the colon. FIG. 15B is a spatiotemporal heatmap of the external forcing function along the length of colon. The vertical lines in FIGS. 15A and 15B correspond to the moment in time when the applied force reaches the proximal end of the colon.

FIGS. 16A-16B: Representative data related to the topology of the particle swarm optimization (PSO). Star topology and neighborhoods of the PSO for 65 particles is shown in FIG. 16A. The contribution of different components in the particle velocity equation as a function of iteration is shown in FIG. 16B. The decay value, d, for FIGS. 16B is 15 iterations.

FIGS. 17A-17H: Representative data related to the optimization process to determine the model parameters to myogenic activity. Cost function for 10 random seeds of particle swarm optimization as a function of iteration count is shown in FIG. 17A. Each line in FIGS. 17A represents the best performance within each independent swarm. FIG. 17B shows the distribution of optimal parameter values (normalized within their minimal and maximal values). The distributions are drawn from 10 independent swarms. A raster plot of smooth muscle action potentials of the best-performing parameters from the optimization with the lowest cost is shown in FIG. 17C. The representative transmembrane potential (Vm) of cyclic bursting behavior in the model (red) and from Yoneda, Takano, Takaki and Suzuki (C. Li, M.-A. Micci, K. S. Murthy, P. J. Pasricha, Substance P is essential for maintaining gut muscle contractility: a novel role for coneurotransmission revealed by botulinum toxin. American Journal of Physiology-Gastrointestinal and Liver Physiology 306, G839-G848 (2014)) (gray) is shown in FIG. 17D. The distribution of mean firing rate (Hz), spikes per burst, interburst frequency (min-1) of bursting myogenic activity, and resting transmembrane potential (Vm) are shown in FIGS. 17E, 17F, 17G and 17H, respectively. Distributions labeled H indicate targets based on extracellular recordings in the mouse colon in the presence of tetrodotoxin, and distributions labeled Y indicate expected values from intracellular recordings in the mouse colon in the presence of tetrodotoxin.

FIGS. 18A-18B: Representative data related to spatiotemporal heatmap segmentation workflow.orkflow for filtering and image preprocessing to segment out the background (red), midground (yellow), and foreground (blue) of the raw, spatiotemporal heatmaps (the example shown is fluorescent calcium). Each heatmap plots the data with the colon length on the y axis (scale 10 mm) and time on the x axis (scale 10 s). Representative calcium fluorescence spatiotemporal heatmap shown as the raw image (left) and the segmented result (right) is shown in FIG. 18B.

FIGS. 19A-19C: Representative data describing how parameters were estimated by particle swarm optimization based on the response to fluid distension. Performance measured as a cost function over iteration is shown in FIG. 19A. Bold lines are the global performance; faint lines indicate the best neighbor performance for the same color-associated swarm. Final cost for each particle, best neighbor, and best global performance for each random seed is shown in FIG. 19B. A circular dendrogram of connections between populations in the model is shown in FIG. 19C. The size of each circle indicates the number of cells in a given population, and the width and color of each connection indicates the number of connections and the mechanism. The drawings use the following abbreviations: ECC, enterochromaffin cell; IPAN, intrinsic primary afferent neuron; ACI, ascending cholinergic interneuron; DNI, descending nitrergic interneuron; DSI, descending somatostatinergic interneuron; EMN, excitatory motor neuron; IMN, inhibitory motor neuron; EGC, enteric glial cell; LMF, longitudinal muscle fiber; CMF, circular muscle fiber; ICC, interstitial cell of Cajal; SMP, submucosal plexus ICC; IML, intramuscular longitudinal ICC; IMC intramuscular circular ICC; MY, myenteric ICC.

FIGS. 20A-20H: Representative data demonstrating how model parameters were estimated to reproduce pellet velocity for increasing pellet diameter. PSO performance for 3 independent swarms (001, 002, & 003) for at least 20 iterations is shown in FIG. 20A. Bold lines depict the best performance for each swarm, and each thin line indicates the best performance for all particles within a given neighborhood (5 neighborhoods per swarm). The cost at PSO termination for each particle (gray dots) is shown in FIG. 20B. The best-performing particles for each neighborhood and the swarm overall are shown in yellow and red, respectively. FIG. 20C shows pellet velocity (mm s⁻¹) as a function of pellet diameter (mm) for the wild-type model (normal serotonin release; red circles) and Tph-/-model (enterochromaffin cells do not release serotonin; yellow circles). For reference, the velocity reported by Heredia et al. (2013) are shown as mean±SEM (raw data were unavailable) for wild-type (red) and Tph-/-mice (yellow). Pellet trajectory for wild-type (red) and Tph-/-(yellow) models with each model repeated with 10 random seeds. The pellet diameter used in each simulation shown in FIGS. 20D-20H was 1.0 mm, 1.5 mm, 2.0 mm, 2.5 mm, and 3.0 mm in FIGS. 20D, 20E, 20F, 20G and 20H, respectively.

FIGS. 21A-21E: Representative data for calcium waves in the isolated mouse colon evoked by pelvic nerve stimulation. Latency, propagation distance and propagation speed of calcium waves evoked by fluid distension and pelvic nerve stimulation in the voided colon and pelvic nerve stimulation in the distended colon are shown in FIGS. 21A, 21B and 21C, respectively. Each color represents independent biological replicates. The rate of calcium waves in the voided and distended colon at baseline (gray), during stimulation (red), and after stimulation (blue) are shown in FIG. 21D (voided) and FIG. 21E (distended). Points connected by faint lines represent one biological replicate. The difference between the frequency of calcium waves during stimulation and the frequency at baseline was normally distributed for both distended and voided colons (n=37, W=0.96, p-value=0.15 and n=45, W=0.97, p-value=0.21).

FIGS. 22A-22D: Representative data demonstrating that the effect of stimulation was dependent on the ongoing rate of calcium waves at baseline. The change in rate as a function of the baseline rate across all conditions, measured as cycles per minute (cpm) is shown in FIG. 22A. Non-negative (blue) and negative (red) changes in the ongoing rate are shown using different colors. Line and shaded region indicate linear regression (y=0.7996−0.4747x, R²=0.192, F-ratio=19.0, p-value<0.0001) and 95% confidence interval of the regression. Logistic fit (blue) demonstrates the likelihood of increasing the rate of calcium waves with stimulation decreased as the baseline rate increased, as shown in FIG. 22B. The ROC curve for predicting the outcome of increasing the rate of calcium waves with stimulation from the baseline rate is shown in FIG. 22C. The red circle indicates the point at which the true positive and false positive rates are maximized and minimized, respectively. The likelihood of successfully increasing the rate of calcium waves with stimulation, as in FIG. 22B, using the threshold criteria identified by the red circle in FIG. 22C is shown in FIG. 22D. The threshold is 0.8 [0.33, 1.25] cpm for predicting the outcome as increasing the frequency from baseline or not.

FIGS. 23A-23D: Representative data of the comparison of fluid distension and pellet propagation in the isolated mouse colon and the computational model. The heatmap of the diameter along the length of colon (vertical scale bar 5 mm) over time (horizontal scale bar 5 s) in the isolated mouse colon and the computational model of colonic motility is shown in FIGS. 23A and 23B, respectively. The heatmap from the model represents the average pixel intensity across five simulations. The stimulus was delivered at the time indicated by the blue line. Grayscale value indicates diameter in mm, consistent across FIGS. 23A and 23B. A heatmap of the change in calcium-associated fluorescence along the length of colon over time in the isolated mouse colon and the computational model is shown in FIGS. 23C and 23D, respectively. The heatmap from the model represents the average pixel intensity across five simulations. The stimulus was delivered at the time indicated by the blue line. Vertical and horizontal axes are consistent with scale bars in FIG. 23D. Colorimetric scale indicates change in fluorescence, consistent between FIGS. 23C and 23D.

FIGS. 24A-24C: Representative data of simulated nerve stimulation in the computational model of colonic motility. Mean pressure response in the rat compared to the pressure response of the computational model is shown in FIG. 24A. The blue horizontal bar indicates the onset and duration of stimulation (100 pulses delivered at 20 Hz). Representative calcium spatiotemporal map from the isolated mouse colon in response to pelvic nerve stimulation (blue vertical line) is shown in FIG. 24B. The calcium spatiotemporal map in response to nerve stimulation in the computational model is shown in FIG. 24C. The heatmaps represent the average pixel intensity across five simulations. The scale bars and colorimetric scale for FIGS. 24A and 24B are consistent.

FIG. 25: Representative data demonstrating that average pellet velocity decreased as the normalized strength of junction potentials decreased.

FIG. 26: Representative data of the pressure time integral of the evoked contraction in the anesthetized rat for increasing burst frequency using 5 s (red) and 40 s (blue) burst durations (n=3, ANOVA F ratio=6.5, p<0.05).

FIG. 27: Representative data of the pressure time integral of the evoked contraction in the computational model for increasing burst duration using 20 Hz stimulation (n=10 model instantiations, F ratio=82.7, p<0.0001).

FIG. 28: Representative data of the pressure time integral of the evoked contraction in the computational model for increasing burst frequency using 5 s (red) and 40 s (blue) burst durations (n=10, F ratio−32.5, p<0.0001).

FIGS. 29A-29C: Representative data of an exemplary neurostimulation system, according to one embodiment of the present disclosure. The neuromodulation system is fully implantable and adapted for sacral nerve stimulation treatment (FIG. 29A) and can include a patient remote and clinician programmer (FIG. 29B). FIG. 29C shows a representative schematic of the architecture of the implantable pulse generator (IPG), according to one embodiment of the present disclosure.

### DETAILED DESCRIPTION

The present disclosure provides systems and methods relating to the bioelectronic modulation of gastrointestinal dysmotility. In particular, the present disclosure provides systems and methods for delivering temporal patterns of electrical stimulation comprising burst-patterned stimulation according to various stimulation parameters to treat gastrointestinal dysmotility disorders in a subject. For example, the present disclosure includes neuromodulation systems and methods that include temporal patterns of electrical stimulation that increase colonic motility. These temporal patterns were validated in an awake rat model of constipation. Further, as described herein, the mechanisms and fundamental limits of colon motor patterns were codified, and this information was used to develop stimulation parameter selection methods. The testing results described in the present disclosure clearly and directly support burst-patterned sacral nerve stimulation with particular parameters as an effective treatment for various gastrointestinal dysmotility disorders (e.g., constipation).

As described further herein, in the isolated mouse colon, burst pelvic nerve stimulation generated substantially more colonic motility than tonic pelvic nerve stimulation. Consistent with the effect of burst-patterned stimulation observed ex vivo, the computational model of colonic motility was also validated, and the computational model was used to demonstrate that burst stimulation of the sacral nerve increased pellet velocity and fecal pellet output more than tonic stimulation of the sacral nerve. Under urethane anesthesia and in a chronic constipation model, burst-patterned sacral nerve stimulation evoked larger and more frequent contractions in the anorectum and increased fecal output when compared to tonic sacral nerve stimulation. These observations confirmed that sacral nerve stimulation delivered in bursts increases colonic motility.

The computational model of motility is the most comprehensive representation of neural control of colonic motility to date. It uniquely incorporates neuronal biophysics and the nuances of neurotransmission mechanisms responsible for fast and slow time constants of colonic motility. The model integrates populations defined by single-cell transcriptomics, mechanosensation among neurons, epithelial cells, pacemaker cells, and muscle fibers, as well as colonic biomechanics, and fluid dynamics to seamlessly recapitulate the sophisticated control system underlying colonic motility. The computational model can also be used to further the optimization of model parameters. The exploration coefficients, swarm size, and decay rate of particle swarm optimization affect when and whether the algorithm converges to a global solution. Future iterations of the computational model can be expanded to other applications, as the outcomes of the model were strongly supported by ex vivo, acute, and chronic in vivo evidence.

Additionally, and as described further herein, anorectal contractions were directly measured and the magnitude of such contractions were compared while systematically varying stimulation frequency, the duration of stimulation, and interburst interval. The minimum effective stimulation frequency, stimulation duration, and interburst interval were identified to maximize anorectal contractions. Previous methods for parameter optimization compared one or two parameters and were limited to varying one parameter at a time, which fail to capture non-linear interactions between parameters. Variations of sacral nerve stimulation parameters have been explored in treating fecal incontinence and urinary incontinence, primarily to prolong battery life. Almost all clinical studies of burst sacral nerve stimulation were conducted in patients with bladder dysfunction. Four studies reported no significant differences in leaks or voids per day between burst and conventional sacral nerve stimulation. One study evaluated the efficacy of burst sacral nerve stimulation (20 s on; 8 s off) in patients with fecal incontinence and found no differences between conventional and burst sacral nerve stimulation. However, there have been no studies to date on the efficacy of burst stimulation of the sacral nerve for the treatment of constipation.

Results of the testing disclosed in the present disclosure demonstrated that burst-patterned nerve stimulation increases prokinetic motility compared to tonic nerve stimulation in the validated computational model, ex vivo mouse colon, and in vivo in rats. For example, burst-patterned sacral nerve stimulation is an effective treatment for slow-transit constipation, and can be applied for either constipation and/or fecal incontinence in a single patient, as these symptoms often present concurrently in disorders of gut-brain interaction. As described further herein, embodiments of the neurostimulation systems and methods disclosed in the present disclosure include real-time control between burst and tonic electrical stimulation as a means for providing dual treatment for both constipation and fecal incontinence, as a bidirectional switch to treat colonic dysmotility. The computational model and test results confirm the application of the testing results described above to human patients.

The computational model and test results support successful translation to human patients where similar results are anticipated. The physiology is highly conserved between rats and humans (Corsetti et al. 2019). Specifically, the duration of the colonic motor complex is consistent between species (Li et al. 2016, Spencer et al. 2012). Both rats and humans exhibit two myogenic pacemaker frequencies, with contractions occurring at high and low frequencies (Rac et al. 1998, Plujá et al. 2001). The similar temporal dynamic properties of colonic motility in the rat and human indicate that similar effects of temporal patterns of stimulation will occur in the human as in the rat.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “temporal pattern” with respect to the application of electrical stimulation (e.g., neuromodulation) generally refers to the timing between stimulation pulses of waveforms. A temporal pattern of electrical stimulation can be applied as part of neuromodulation therapy to a subject in need thereof using various stimulation parameters, including but not limited to, stimulation pulse amplitude, stimulation pulse width, stimulation pulse frequency, stimulation pulse waveform shape, and/or ramp time.

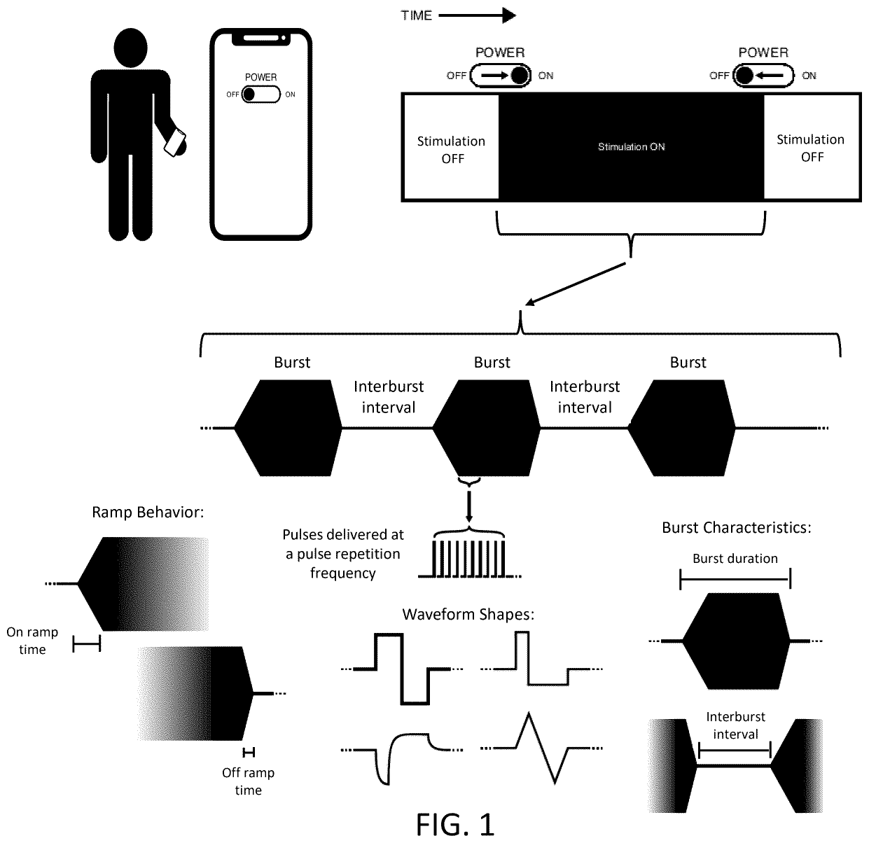

As used herein, “burst-patterned electrical stimulation,” and “a burst pattern of electrical stimulation” generally refers to a type of temporal pattern of electrical stimulation that alternates between delivering for a first amount of time electrical current according to the shape of a programmed waveform and various stimulation parameters, and delivering no current for a second amount of time (e.g., application of bursts of electrical current as part of neuromodulation therapy). As described further herein, burst-patterned stimulation delivers a programmed waveform according to various stimulation parameters, including but not limited to, pulse amplitude, pulse width, pulse shape, pulse repetition frequency, burst duration, interburst interval, and ramp time. In some aspects, the pulse is repeated at a pulse repetition frequency for a period of time defined by the burst duration, which can include on ramp time and off ramp time; and, the interburst interval defines the period of time when no current is being delivered (see, e.g., FIG. 1). In some aspects, the pulse repetition frequency can be considered the inverse of the period of time between the start of individual pulses. And, similarly, the burst repetition frequency can be considered the inverse of the period of time between the start of individual bursts. The pulse shape can include, but is not limited to, a rectangular shape, a sinusoidal shape, a ramp, an exponential rise, and/or an exponential fall. For example, the waveform shape can be a biphasic square wave with a programmed amplitude and pulse width. In some embodiments, the amplitude of a waveform may be scaled up when transitioning from delivering no current, to delivering current for a period of time defined by the programmed on ramp time; and the amplitude of the waveform may be scaled down when transitioning from delivering current, to delivering no current for a period of time defined by the programmed off ramp time.

Burst-patterned stimulation is distinct from continuous stimulation because during burst-patterned stimulation there is a period of time during which no current is delivered. This period is defined by the interburst interval. Although both burst-patterned stimulation and continuous stimulation can be cycled on and off, burst-patterned stimulation includes at least one interburst interval that is repeated in each cycle, which is distinct from continuous stimulation.

As used herein, “gastrointestinal motility” and “gastrointestinal dysmotility” as used herein generally refer to the motility and contractions of the digestive system and the transit of the contents within it. Accordingly, when nerves and/or muscles in any portion of the digestive tract do not function normally (e.g., a hypermotility or a hypomotility disorder), a subject can develop one or more symptoms related to gut dysmotility. Symptoms of gastrointestinal dysmotility disorder include, but are not limited to, early satiety, nausea, vomiting, bloating, diarrhea, constipation, involuntary weight loss, abdominal pain, abdominal swelling (distention), and/or intrarectal pressure. As described further herein, a gastrointestinal dysmotility disorder can include colonic dysmotility (e.g., constipation or chronic constipation).

As used herein, “subject” and “patient” generally refer to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject is a human. The subject or patient may be undergoing various forms of treatment (e.g., neuromodulation therapy).

As used herein, “treat,” “treating,” and “treatment” generally refer to reversing, alleviating, or inhibiting the progress of a disease and/or disorder, or one or more symptoms of such disease or disorder, to which such terms apply. Depending on the condition of the subject, the term also refers to preventing a disease or disorder and includes preventing the onset of a disease or disorder, or preventing the symptoms associated with a disease or disorder. A treatment may be either performed in an acute or chronic way. Treatment and related terms can also refer to reducing the severity of a disease or disorder, or symptoms associated with such disease or disorder, prior to manifestation of the disease or disorder. In some aspects, treating one or more symptoms of a disease or disorder includes providing a degree of relief of one or more symptoms of the disease or disorder.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities, and plural terms shall include the singular.

### 2. Methods of Treatment

The present disclosure provides systems and methods relating to the neuromodulation of gastrointestinal dysmotility. In particular, the present disclosure provides systems and methods for delivering temporal patterns of electrical stimulation comprising burst-patterned stimulation according to various stimulation parameters to treat gastrointestinal dysmotility disorders in a subject. In accordance with these embodiments, the present disclosure includes methods and systems for treating a gastrointestinal dysmotility disorder in a subject in need thereof. In some embodiments, the method includes applying a temporal pattern of electrical stimulation comprising burst-patterned stimulation to a target nerve or a set of target nerves in a subject having at least one symptom of a gastrointestinal dysmotility disorder, such that the application of the temporal pattern of electrical stimulation modulates gastrointestinal motility in the subject. The methods of the present disclosure include application of a temporal pattern of electrical stimulation to a subject according to one or more general stimulation parameters. These stimulation patterns include, but are not limited to, stimulation pulse amplitude, stimulation pulse width, stimulation pulse frequency, stimulation pulse waveform shape, and/or ramp time. As described further herein, a temporal pattern with respect to the application of electrical stimulation (e.g., neuromodulation) generally refers to the timing between stimulation pulses of waveforms. A temporal pattern of electrical stimulation can be applied as part of neuromodulation therapy to a subject in need thereof using various stimulation parameters, including but not limited to, stimulation pulse amplitude, stimulation pulse width, stimulation pulse frequency, stimulation pulse waveform shape, and ramp time.

In some embodiments, burst-patterned electrical stimulation refers to a type of temporal pattern of electrical stimulation that alternates between delivering for a first amount of time electrical current according to the shape of a programmed waveform and various stimulation parameters, and delivering no current for a second amount of time (e.g., application of bursts of electrical current as part of neuromodulation therapy). As described further herein, burst-patterned stimulation delivers a programmed waveform according to various stimulation parameters, including but not limited to, pulse amplitude, pulse width, pulse shape, pulse repetition frequency, burst duration, interburst interval, and ramp time. In some aspects, the pulse is repeated at a pulse repetition frequency for a period of time defined by the burst duration, which can include on ramp time and off ramp time; and, the interburst interval defines the period of time when no current is being delivered (see, e.g., FIG. 1). The pulse shape can include, but is not limited to, a rectangular shape, a sinusoidal shape, a ramp, an exponential rise, and/or an exponential fall. For example, the waveform shape can be a biphasic square wave with a programmed amplitude and pulse width. In some embodiments, the amplitude of a waveform may be scaled up when transitioning from delivering no current, to delivering current for a period of time defined by the programmed on ramp time; and the amplitude of the waveform may be scaled down when transitioning from delivering current, to delivering no current for a period of time defined by the programmed off ramp time. Burst-patterned stimulation is distinct from continuous stimulation because during burst-patterned stimulation there is a period of time during which no current is delivered. This period is defined as the interburst interval (see, e.g., FIG. 1). Although both burst-patterned stimulation and continuous stimulation can be cycled on and off, burst-patterned stimulation includes at least one interburst interval that is repeated in each cycle, which is distinct from continuous stimulation.

In accordance with these embodiments, methods of applying neuromodulation treatment to a subject includes, but is not limited to, applying burst-patterned stimulation comprising at least two identical bursts. In some embodiments, neuromodulation treatment includes applying burst-patterned stimulation comprising at least two non-identical bursts. Various combinations of identical and non-identical bursts can be applied to a subject as part of neuromodulation treatment based on an assessment of the subject's symptoms and determining an efficacious stimulation pattern to treat those symptoms.

In some embodiments, the neuromodulation methods of the present disclosure include applying burst-patterned stimulation at a certain pulse repetition frequency. Pulse repetition frequency (the number of pulses of a repeating signal per unit of time) for each burst duration can range from about 0.1 Hz to about 30 Hz. In some embodiments, the pulse repetition frequency ranges from about 1.0 Hz to about 30 Hz. In some embodiments, the pulse repetition frequency ranges from about 5.0 Hz to about 30 Hz. In some embodiments, the pulse repetition frequency ranges from about 10 Hz to about 30 Hz. In some embodiments, the pulse repetition frequency ranges from about 15 Hz to about 30 Hz. In some embodiments, the pulse repetition frequency ranges from about 20 Hz to about 30 Hz. In some embodiments, the pulse repetition frequency ranges from about 25 Hz to about 30 Hz. In some embodiments, the pulse repetition frequency ranges from about 0.1 Hz to about 25 Hz. In some embodiments, the pulse repetition frequency ranges from about 0.1 Hz to about 20 Hz. In some embodiments, the pulse repetition frequency ranges from about 0.1 Hz to about 15 Hz. In some embodiments, the pulse repetition frequency ranges from about 0.1 Hz to about 10 Hz. In some embodiments, the pulse repetition frequency ranges from about 0.1 Hz to about 5 Hz. In some embodiments, the pulse repetition frequency ranges from about 5 Hz to about 25 Hz. In some embodiments, the pulse repetition frequency ranges from about 6 Hz to about 25 Hz. In some embodiments, the pulse repetition frequency ranges from about 7 Hz to about 25 Hz. In some embodiments, the pulse repetition frequency ranges from about 8 Hz to about 25 Hz. In some embodiments, the pulse repetition frequency ranges from about 9 Hz to about 25 Hz. In some embodiments, the pulse repetition frequency ranges from about 10 Hz to about 25 Hz. In some embodiments, the pulse repetition frequency ranges from about 10 Hz to about 20 Hz. In some embodiments, the pulse repetition frequency ranges from about 15 Hz to about 25 Hz. In some embodiments, the pulse repetition frequency ranges from about 5 Hz to about 24 Hz. In some embodiments, the pulse repetition frequency ranges from about 5 Hz to about 23 Hz. In some embodiments, the pulse repetition frequency ranges from about 5 Hz to about 22 Hz. In some embodiments, the pulse repetition frequency ranges from about 5 Hz to about 21 Hz. In some embodiments, the pulse repetition frequency ranges from about 5 Hz to about 20 Hz. In some embodiments, the pulse repetition frequency ranges from about 8 Hz to about 20 Hz. In some embodiments, the pulse repetition frequency ranges from about 8 Hz to about 16 H2.

In some embodiments, the neuromodulation methods of the present disclosure include applying burst-patterned stimulation for a certain burst duration. In some embodiments, a pulse (or waveform) is repeated at a repetition frequency for a period of time defined by the burst duration, which can include on ramp time and off ramp time (see, e.g., FIG. 1). Burst duration can range from about 10 seconds to about 60 seconds. In some embodiments, the burst duration ranges from about 15 seconds to about 60 seconds. In some embodiments, the burst duration ranges from about 20 seconds to about 60 seconds. In some embodiments, the burst duration ranges from about 25 seconds to about 60 seconds. In some embodiments, the burst duration ranges from about 30 seconds to about 60 seconds. In some embodiments, the burst duration ranges from about 35 seconds to about 60 seconds. In some embodiments, the burst duration ranges from about 40 seconds to about 60 seconds. In some embodiments, the burst duration ranges from about 45 seconds to about 60 seconds. In some embodiments, the burst duration ranges from about 50 seconds to about 60 seconds. In some embodiments, the burst duration ranges from about 55 seconds to about 60 seconds. In some embodiments, the burst duration ranges from about 10 seconds to about 55 seconds. In some embodiments, the burst duration ranges from about 10 seconds to about 50 seconds. In some embodiments, the burst duration ranges from about 10 seconds to about 45 seconds. In some embodiments, the burst duration ranges from about 10 seconds to about 40 seconds. In some embodiments, the burst duration ranges from about 10 seconds to about 35 seconds. In some embodiments, the burst duration ranges from about 10 seconds to about 30 seconds. In some embodiments, the burst duration ranges from about 10 seconds to about 25 seconds. In some embodiments, the burst duration ranges from about 10 seconds to about 20 seconds. In some embodiments, the burst duration ranges from about 10 seconds to about 15 seconds. In some embodiments, the burst duration ranges from about 10 seconds to about 50 seconds. In some embodiments, the burst duration ranges from about 15 seconds to about 45 seconds. In some embodiments, the burst duration ranges from about 20 seconds to about 40 seconds. In some embodiments, the burst duration ranges from about 15 seconds to about 30 seconds. In some embodiments, the burst duration ranges from about 25 seconds to about 50 seconds. In some embodiments, the burst duration ranges from about 30 seconds to about 50 seconds.

In some embodiments, the neuromodulation methods of the present disclosure include applying burst-patterned stimulation comprising a certain interburst interval. In some embodiments, the interburst interval defines the period of time when no current is being delivered (see, e.g., FIG. 1). In some embodiments, burst-patterned stimulation includes an interburst interval between burst durations, and the interburst interval is not dependent on the length of a refractory period of a subject's colon. In some embodiments, the interburst interval ranges from about 10 seconds to about 120 seconds. In some embodiments, the interburst interval ranges from about 20 seconds to about 120 seconds. In some embodiments, the interburst interval ranges from about 30 seconds to about 120 seconds. In some embodiments, the interburst interval ranges from about 40 seconds to about 120 seconds. In some embodiments, the interburst interval ranges from about 50 seconds to about 120 seconds. In some embodiments, the interburst interval ranges from about 60 seconds to about 120 seconds. In some embodiments, the interburst interval ranges from about 70 seconds to about 120 seconds. In some embodiments, the interburst interval ranges from about 80 seconds to about 120 seconds. In some embodiments, the interburst interval ranges from about 90 seconds to about 120 seconds. In some embodiments, the interburst interval ranges from about 100 seconds to about 120 seconds. In some embodiments, the interburst interval ranges from about 110 seconds to about 120 seconds. In some embodiments, the interburst interval ranges from about 10 seconds to about 110 seconds. In some embodiments, the interburst interval ranges from about 10 seconds to about 100 seconds. In some embodiments, the interburst interval ranges from about 10 seconds to about 90 seconds. In some embodiments, the interburst interval ranges from about 10 seconds to about 80 seconds. In some embodiments, the interburst interval ranges from about 10 seconds to about 70 seconds. In some embodiments, the interburst interval ranges from about 10 seconds to about 60 seconds. In some embodiments, the interburst interval ranges from about 10 seconds to about 50 seconds. In some embodiments, the interburst interval ranges from about 10 seconds to about 40 seconds. In some embodiments, the interburst interval ranges from about 10 seconds to about 30 seconds. In some embodiments, the interburst interval ranges from about 10 seconds to about 20 seconds. In some embodiments, the interburst interval ranges from about 30 seconds to about 100 seconds. In some embodiments, the interburst interval ranges from about 40 seconds to about 90 seconds. In some embodiments, the interburst interval ranges from about 50 seconds to about 80 seconds. In some embodiments, the interburst interval ranges from about 20 seconds to about 80 seconds. In some embodiments, the interburst interval ranges from about 60 seconds to about 90 seconds. In some embodiments, the interburst interval ranges from about 30 seconds to about 50 seconds. In some embodiments, the interburst interval ranges from about 30 seconds to about 60 seconds.

In some embodiments, the neuromodulation methods of the present disclosure include applying burst-patterned stimulation according to a certain pulse or waveform shape. The waveform shape can include, but is not limited to, a rectangular shape, a sinusoidal shape, a ramp, an exponential rise, and/or an exponential fall (see, e.g., FIG. 1). In some embodiments, the pulse shape is monophasic or biphasic. For example, the waveform shape can be a biphasic square wave with a programmed amplitude and pulse width. In some embodiments, the amplitude of a waveform may be scaled up when transitioning from delivering no current, to delivering current for a period of time defined by the programmed on ramp time; and the amplitude of the waveform may be scaled down when transitioning from delivering current, to delivering no current for a period of time defined by the programmed off ramp time.

As described further herein, the neuromodulation methods of the present disclosure can be used to treat a gastrointestinal motility/dysmotility disorder in a subject. A gastrointestinal motility disorder or a gastrointestinal dysmotility disorder, as used herein, generally refer to the motility and contractions of the digestive system and the transit of the contents within it. Accordingly, when nerves and/or muscles in any portion of the digestive tract do not function normally (e.g., a hypermotility or a hypomotility disorder), a subject can develop one or more symptoms related to gut dysmotility. Symptoms of gastrointestinal dysmotility disorder include, but are not limited to, early satiety, nausea, vomiting, bloating, diarrhea, constipation, involuntary weight loss, abdominal pain, abdominal swelling (distention), and/or intrarectal pressure. In some embodiments, the neuromodulation methods of the present disclosure can be used to treat a hypomotility disorder. In some embodiments, the neuromodulation methods of the present disclosure can be used to treat a hypermotility disorder. In some embodiments, the neuromodulation methods of the present disclosure can be used to treat colonic dysmotility. In some embodiments, the neuromodulation methods of the present disclosure can be used to treat constipation.

In some embodiments, the methods of the present disclosure can be used to treat more than one gastrointestinal motility/dysmotility in a subject, for example, by alternating the type of temporal pattern of stimulation applied. In some embodiments, a temporal pattern of electrical stimulation can be applied to a subject that includes burst-patterned stimulation as well as at least one additional temporal pattern of electrical stimulation. For example, in some embodiments, the additional temporal pattern of electrical stimulation is a second burst-patterned stimulation (e.g., comprising different stimulation patterns as compared to the first burst-patterned stimulation).

As described further herein, the neuromodulation methods of the present disclosure target a nerve or set of nerves in a subject having or suspected of having a gastrointestinal dysmotility disorder. In some embodiments, the target nerve or set of target nerves comprises an extrinsic nerve or set of extrinsic nerves, or intrinsic (enteric) nerves. In some embodiments, the extrinsic nerve or set of extrinsic nerves comprise vagal afferent or vagal efferent nerves, splanchnic nerves, pelvic nerves, rectal nerves, lumbar colonic nerves, hypogastric verves, and/or sacral nerves. In some embodiments, the intrinsic nerves comprise nerves that lie within the wall of the gastrointestinal tract. In some embodiments, the extrinsic nerve or set of extrinsic nerves, or the intrinsic (enteric) nerves innervate the gastrointestinal tract. In some embodiments, the target nerve or a set of target nerves comprises the sacral nerve, and wherein the gastrointestinal dysmotility disorder comprises constipation. In some embodiments, the target nerve or a set of target nerves comprises the sacral nerve, and wherein the gastrointestinal dysmotility disorder comprises disorders of gut-brain interactions.

The neuromodulation methods of the present disclosure also include treating a gastrointestinal dysmotility disorder in a human subject in need thereof by programming a pulse generator to output a temporal pattern of electrical stimulation comprising burst-patterned stimulation to a target nerve or set of target nerves in the subject having at least one symptom of a gastrointestinal dysmotility disorder. In accordance with these methods, embodiments of the present disclosure include delivering the temporal pattern of electrical stimulation to the subject, such that delivering the temporal pattern of electrical stimulation modulates motility in the subject and thereby treats the gastrointestinal dysmotility disorder. In some embodiments, the at least one temporal pattern of electrical stimulation comprising the burst-patterned stimulation is delivered according to stimulation parameters determined to treat the gastrointestinal dysmotility disorder. In some embodiments, the burst-patterned stimulation is applied according to one or more stimulation parameters comprising pulse amplitude, pulse width, pulse shape, pulse repetition frequency, burst duration, interburst interval, and ramp time. In some embodiments, the pulse generator is configured to output the temporal pattern of electrical stimulation comprising the burst-patterned stimulation and at least one additional temporal pattern of electrical stimulation. In some embodiments, the pulse generator is configured to allow the subject to alternate between delivering the temporal pattern of electrical stimulation comprising the burst-patterned stimulation and the at least one additional temporal pattern of electrical stimulation. In some embodiments, the additional temporal pattern of electrical stimulation comprises a second burst-patterned stimulation.

Embodiments of the present disclosure also include a method of selecting a temporal pattern of electrical stimulation to treat a gastrointestinal dysmotility disorder in a human subject in need thereof. In accordance with these embodiments, the method includes delivering a first burst-patterned stimulation to a target nerve or a set of target nerves in a subject having at least one symptom of a gastrointestinal dysmotility disorder and assessing efficacy of stimulation and/or a degree of relief of the at least one symptom. In some embodiments, the method includes determining a second burst-patterned stimulation by adjusting a stimulation parameter of the first burst-patterned stimulation, and delivering the second burst-patterned stimulation to the target nerve or the set of target nerves in the subject and reassessing the efficacy of stimulation and/or the degree of relief of the at least one symptom. In some embodiments, the method includes selecting for treatment one of the first burst-patterned stimulation or the second burst-patterned stimulation based on the efficacy of stimulation and/or the degree of relief.

In some embodiments, assessing and reassessing the efficacy of stimulation and/or the degree of relief involves measuring intrarectal pressure (e.g., using anorectal manometry). In some embodiments, the stimulation parameter comprises one or more of pulse amplitude, pulse width, pulse shape, pulse repetition frequency, burst duration, interburst interval, and/or ramp time. In some embodiments, the method further comprises adjusting a second stimulation parameter. In some embodiments, the method further comprises readjusting the adjusted stimulation parameter.

In some embodiments, the at least one symptom comprises early satiety, nausea, vomiting, bloating, diarrhea, constipation, involuntary weight loss, abdominal pain, abdominal swelling (distention), and/or intrarectal pressure. In some embodiments, assessment of the at least one symptom comprises measurement of intrarectal pressure. In some embodiments, and intrarectal pressure is measured using anorectal manometry. In some embodiments, assessing the efficacy of stimulation and/or the degree of relief of the at least one symptom comprises determining a paired-burst response ratio corresponding to quantification of the first response to the first burst-patterned stimulation as compared to quantification of the second response of the second burst-patterned stimulation. In some embodiments, the method selects a first treatment for at least one symptom of a first gastrointestinal dysmotility disorder, and the method is repeated to select at least a second treatment for at least one symptom of a second gastrointestinal disorder.

### 3. Neuromodulation Systems

Embodiments of the present disclosure also include a system for treating gastrointestinal dysmotility disorder in a subject in need thereof. In accordance with these embodiments, the system includes a pulse generator that includes a processor, a lead electrically coupled to the device, and an electrode electrically coupled to the lead and positioned to transmit an electrical stimulation signal to a target nerve or set of target nerves in the subject. In some embodiments, the processor is configured to control the pulse generator to provide the electrical stimulation signal to the target nerve or the set of target nerves in the subject in a first temporal pattern comprising burst-patterned stimulation. In some embodiments, the application of the first temporal pattern modulates gastrointestinal motility in the subject, thereby treating the gastrointestinal dysmotility disorder. In some embodiments, the gastrointestinal motility disorder comprises constipation.

In some embodiments, the pulse generator is configured for implantation into a human subject. In some embodiments, the system further comprises a remote control device that is configured to control the implantable pulse generator and the electrical stimulation signal being provided to the target nerve or set of target nerves. In some embodiments, the remote control device is in wireless communication with the pulse generator. In some embodiments, the processor is configured to adjust one or more stimulation parameters of the electrical stimulation signal in response to user input received at the remote control device. In some embodiments, the pulse generator is cycled off in response to user input received at the remote control device. In some embodiments, the system further includes a remote control device that is configured to control the pulse generator.

In some embodiments, the remote control device is configured to receive a user input to select the first temporal pattern or the second temporal pattern for the electrical stimulation signal being provided to the target nerve or set of target nerves. In some embodiments, the system further includes a programmer in communication with the pulse generator and configured to control the processor to modify one or more stimulation parameters of the electrical stimulation signal. In some embodiments, the first temporal pattern includes an interburst interval between burst durations during which the burst-patterned stimulation is provided, and the interburst interval is not dependent on the length of a refractory period of the subject's colon.

In some embodiments, the neuromodulation systems of the present disclosure can be configured to apply burst-patterned stimulation at a certain pulse repetition frequency (see, e.g., FIG. 1). Pulse repetition frequency (the number of pulses of a repeating signal per unit of time) for each burst duration can range from about 0.1 Hz to about 30 Hz. In some embodiments, the pulse repetition frequency ranges from about 1.0 Hz to about 30 Hz. In some embodiments, the pulse repetition frequency ranges from about 5.0 Hz to about 30 Hz. In some embodiments, the pulse repetition frequency ranges from about 10 Hz to about 30 Hz. In some embodiments, the pulse repetition frequency ranges from about 15 Hz to about 30 Hz. In some embodiments, the pulse repetition frequency ranges from about 20 Hz to about 30 Hz. In some embodiments, the pulse repetition frequency ranges from about 25 Hz to about 30 Hz. In some embodiments, the pulse repetition frequency ranges from about 0.1 Hz to about 25 Hz. In some embodiments, the pulse repetition frequency ranges from about 0.1 Hz to about 20 Hz. In some embodiments, the pulse repetition frequency ranges from about 0.1 Hz to about 15 Hz. In some embodiments, the pulse repetition frequency ranges from about 0.1 Hz to about 10 Hz. In some embodiments, the pulse repetition frequency ranges from about 0.1 Hz to about 5 Hz. In some embodiments, the pulse repetition frequency ranges from about 5 Hz to about 25 Hz. In some embodiments, the pulse repetition frequency ranges from about 6 Hz to about 25 Hz. In some embodiments, the pulse repetition frequency ranges from about 7 Hz to about 25 Hz. In some embodiments, the pulse repetition frequency ranges from about 8 Hz to about 25 Hz. In some embodiments, the pulse repetition frequency ranges from about 9 Hz to about 25 Hz. In some embodiments, the pulse repetition frequency ranges from about 10 Hz to about 25 Hz. In some embodiments, the pulse repetition frequency ranges from about 10 Hz to about 20 Hz. In some embodiments, the pulse repetition frequency ranges from about 15 Hz to about 25 Hz. In some embodiments, the pulse repetition frequency ranges from about 5 Hz to about 24 Hz. In some embodiments, the pulse repetition frequency ranges from about 5 Hz to about 23 Hz. In some embodiments, the pulse repetition frequency ranges from about 5 Hz to about 22 Hz. In some embodiments, the pulse repetition frequency ranges from about 5 Hz to about 21 Hz. In some embodiments, the pulse repetition frequency ranges from about 5 Hz to about 20 Hz. In some embodiments, the pulse repetition frequency ranges from about 8 Hz to about 20 Hz. In some embodiments, the pulse repetition frequency ranges from about 8 Hz to about 16 Hz.

The neuromodulation systems of the present disclosure can be configured to apply burst-patterned stimulation for a certain burst duration. In some embodiments, a pulse (or waveform) is repeated at a repetition frequency for a period of time defined by the burst duration, which can include on ramp time and off ramp time (see, e.g., FIG. 1). Burst duration can range from about 10 seconds to about 60 seconds. In some embodiments, the burst duration ranges from about 15 seconds to about 60 seconds. In some embodiments, the burst duration ranges from about 20 seconds to about 60 seconds. In some embodiments, the burst duration ranges from about 25 seconds to about 60 seconds. In some embodiments, the burst duration ranges from about 30 seconds to about 60 seconds. In some embodiments, the burst duration ranges from about 35 seconds to about 60 seconds. In some embodiments, the burst duration ranges from about 40 seconds to about 60 seconds. In some embodiments, the burst duration ranges from about 45 seconds to about 60 seconds. In some embodiments, the burst duration ranges from about 50 seconds to about 60 seconds. In some embodiments, the burst duration ranges from about 55 seconds to about 60 seconds. In some embodiments, the burst duration ranges from about 10 seconds to about 55 seconds. In some embodiments, the burst duration ranges from about 10 seconds to about 50 seconds. In some embodiments, the burst duration ranges from about 10 seconds to about 45 seconds. In some embodiments, the burst duration ranges from about 10 seconds to about 40 seconds. In some embodiments, the burst duration ranges from about 10 seconds to about 35 seconds. In some embodiments, the burst duration ranges from about 10 seconds to about 30 seconds. In some embodiments, the burst duration ranges from about 10 seconds to about 25 seconds. In some embodiments, the burst duration ranges from about 10 seconds to about 20 seconds. In some embodiments, the burst duration ranges from about 10 seconds to about 15 seconds. In some embodiments, the burst duration ranges from about 10 seconds to about 50 seconds. In some embodiments, the burst duration ranges from about 15 seconds to about 45 seconds. In some embodiments, the burst duration ranges from about 20 seconds to about 40 seconds. In some embodiments, the burst duration ranges from about 15 seconds to about 30 seconds. In some embodiments, the burst duration ranges from about 25 seconds to about 50 seconds.

The neuromodulation systems of the present disclosure can be configured to apply burst-patterned stimulation comprising a certain interburst interval. In some embodiments, the interburst interval defines the period of time when no current is being delivered (see, e.g., FIG. 1). In some embodiments, burst-patterned stimulation includes an interburst interval between burst durations, and the interburst interval is not dependent on the length of a refractory period of a subject's colon. In some embodiments, the interburst interval ranges from about 10 seconds to about 120 seconds. In some embodiments, the interburst interval ranges from about 20 seconds to about 120 seconds. In some embodiments, the interburst interval ranges from about 30 seconds to about 120 seconds. In some embodiments, the interburst interval ranges from about 40 seconds to about 120 seconds. In some embodiments, the interburst interval ranges from about 50 seconds to about 120 seconds. In some embodiments, the interburst interval ranges from about 60 seconds to about 120 seconds. In some embodiments, the interburst interval ranges from about 70 seconds to about 120 seconds. In some embodiments, the interburst interval ranges from about 80 seconds to about 120 seconds. In some embodiments, the interburst interval ranges from about 90 seconds to about 120 seconds. In some embodiments, the interburst interval ranges from about 100 seconds to about 120 seconds. In some embodiments, the interburst interval ranges from about 110 seconds to about 120 seconds. In some embodiments, the interburst interval ranges from about 10 seconds to about 110 seconds. In some embodiments, the interburst interval ranges from about 10 seconds to about 100 seconds. In some embodiments, the interburst interval ranges from about 10 seconds to about 90 seconds. In some embodiments, the interburst interval ranges from about 10 seconds to about 80 seconds. In some embodiments, the interburst interval ranges from about 10 seconds to about 70 seconds. In some embodiments, the interburst interval ranges from about 10 seconds to about 60 seconds. In some embodiments, the interburst interval ranges from about 10 seconds to about 50 seconds. In some embodiments, the interburst interval ranges from about 10 seconds to about 40 seconds. In some embodiments, the interburst interval ranges from about 10 seconds to about 30 seconds. In some embodiments, the interburst interval ranges from about 10 seconds to about 20 seconds. In some embodiments, the interburst interval ranges from about 30 seconds to about 100 seconds. In some embodiments, the interburst interval ranges from about 40 seconds to about 90 seconds. In some embodiments, the interburst interval ranges from about 50 seconds to about 80 seconds. In some embodiments, the interburst interval ranges from about 20 seconds to about 80 seconds. In some embodiments, the interburst interval ranges from about 60 seconds to about 90 seconds. In some embodiments, the interburst interval ranges from about 30 seconds to about 50 seconds.

The neuromodulation systems of the present disclosure can be configured to apply burst-patterned stimulation according to a certain pulse or waveform shape. The waveform shape can include, but is not limited to, a rectangular shape, a sinusoidal shape, a ramp, an exponential rise, and/or an exponential fall (see, e.g., FIG. 1). In some embodiments, the pulse shape is monophasic or biphasic. For example, the waveform shape can be a biphasic square wave with a programmed amplitude and pulse width. In some embodiments, the amplitude of a waveform may be scaled up when transitioning from delivering no current, to delivering current for a period of time defined by the programmed on ramp time; and the amplitude of the waveform may be scaled down when transitioning from delivering current, to delivering no current for a period of time defined by the programmed off ramp time.

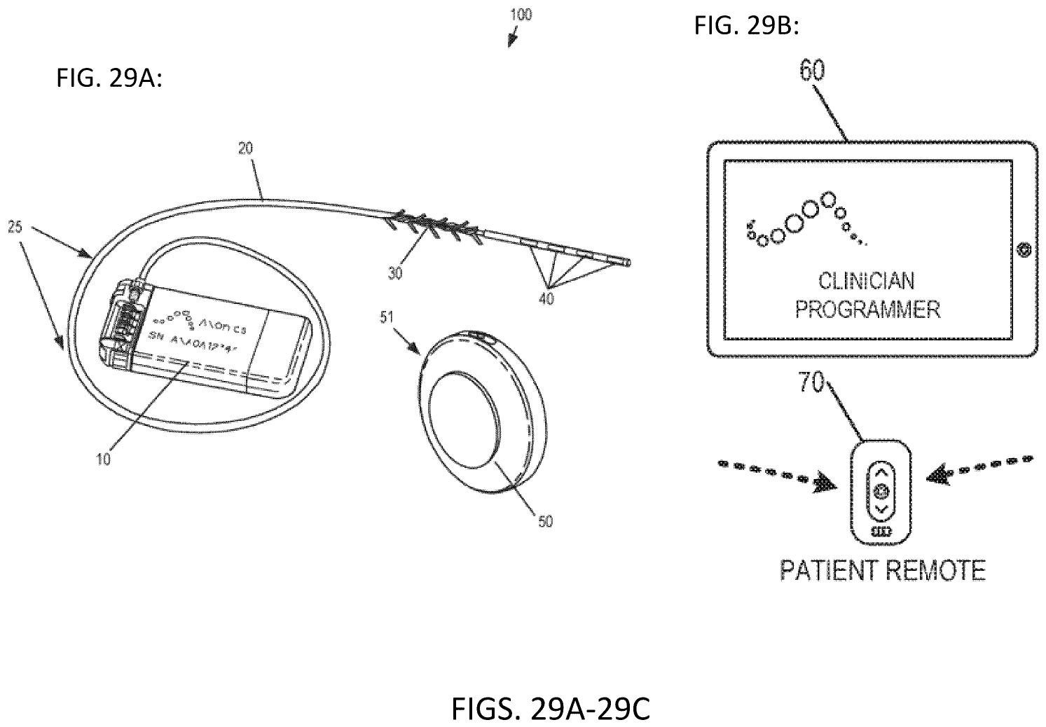

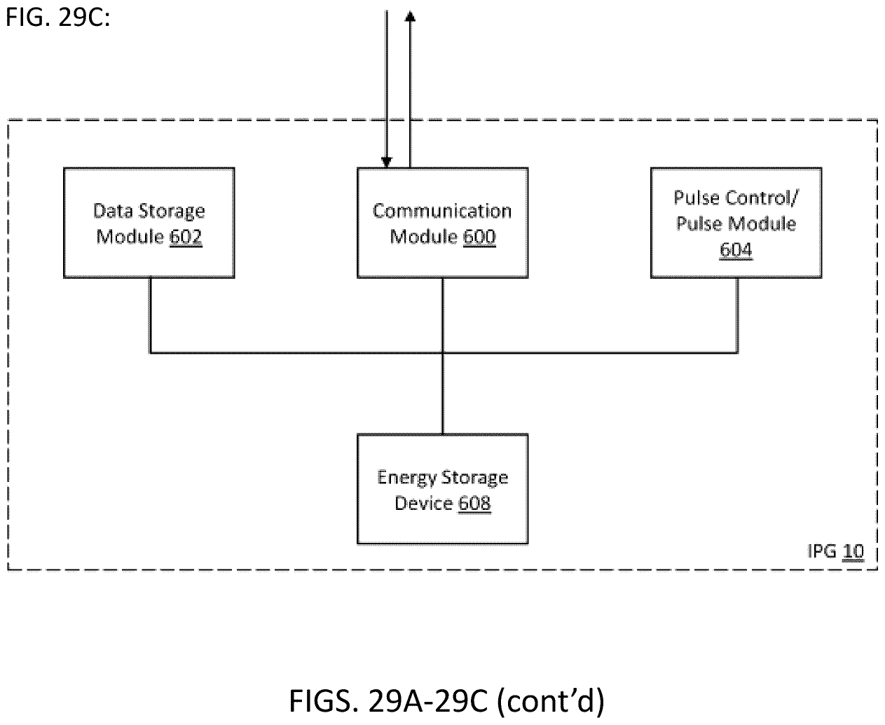

In accordance with the above embodiments, FIGS. 29A-29C provide one embodiment of a neurostimulation system of the present disclosure. FIG. 29A illustrates an example neurostimulation system 100 that is fully implantable and adapted for sacral nerve stimulation treatment. The implantable system 100 includes an implantable pulse generator (IPG) 10 that is coupled to a neurostimulation lead 20 that includes a group of neurostimulation electrodes 40 at a distal end of the lead (FIG. 29A). The lead includes a lead anchor portion 30 with a series of tines extending radially outward so as to anchor the lead and maintain a position of the neurostimulation lead 20 after implantation. The lead 20 may further include one or more radiopaque markers 25 to assist in locating and positioning the lead using visualization techniques such as fluoroscopy. In some embodiments, the IPG provides monopolar or bipolar electrical pulses that are delivered to the targeted nerves through one or more neurostimulation electrodes, typically four electrodes. In sacral nerve stimulation, the lead is typically implanted through the S3 foramen as described herein.

The IPG may be non-rechargeable or rechargeable. A rechargeable IPG may be configured to be rechargeable wirelessly through conductive coupling by use of a charging device 50 (CD), which is a portable device powered by a rechargeable battery to allow patient mobility while charging. The CD is used for transcutaneous charging of the IPG through RF induction. The CD can either be either patched to the patient's skin using an adhesive or can be held in place using a belt 53 or by an adhesive patch 52. The CD may be charged by plugging the CD directly into an outlet or by placing the CD in a charging dock or station 51 that connects to an AC wall outlet or other power source.

The system may further include a patient remote 70 and clinician programmer 60 (FIG. 29B), each configured to wirelessly communicate with the implanted IPG, or with the FPG during a trial. The clinician programmer 60 may be a tablet computer used by the clinician to program the IPG and the FPG. The device also has the capability to record stimulation-induced electromyograms (EMGs) to facilitate lead placement, programming, and/or re-programming. The patient remote may be a battery-operated, portable device that utilizes radio-frequency (RF) signals to communicate with the FPG and IPG and allows the patient to adjust the stimulation levels, check the status of the IPG battery level, and/or to turn the stimulation on or off.

Additionally, FIG. 29C shows a representative schematic of the architecture of the implantable pulse generator (IPG), according to one embodiment of the present disclosure. In some embodiments, each of the components of the architecture of the IPG 10 may be implemented using the processor, memory, and/or other hardware components of the IPG 10. In some embodiments, the components of the architecture of the IPG 10 may include software that interacts with the hardware of the IPG 10 to achieve a desired outcome, and the components of the architecture of the IPG 10 may be located within the housing.

In some embodiments, the IPG 10 may include, for example, a communication module 600. The communication module 600 may be configured to send data to and receive data from other components and/or devices of the exemplary nerve stimulation system including, for example, the clinician programmer 60 and/or the patient remote 70. In some embodiments, the communication module 600 may include one or several antennas and software configured to control the one or several antennas to send information to and receive information from one or several of the other components of the IPG 10.

In some embodiments, the communication module 600 may be configured to operate in a plurality of modes, which may include (but are not limited to) a detect mode, a receive mode, and a data transfer mode. In one embodiment, the communication module 600 may operate in detect mode by transmitting a detection burst. In one embodiment, this detection burst may be followed by a sleep period. In one embodiment, the detection burst may be included in a plurality of detection bursts, wherein the plurality of detection bursts is configured to provide detection bursts across a frequency spectrum. In one embodiment, the communication module 600 may operate in receive mode by confirming a detection of a communication channel, identifying the communication channel, and locking the preamble for subsequent data communications with the communication channel. The communication channel may be a communication channel between the communication module 600 and other components of the IPG 10, or between the communication module 600 and outside devices such as the clinician programmer 60 and/or the patient remote 70. In one embodiment, the communication module 600 may operate in data transfer mode by using at least one ON-period to receive discreet packets of data across the communication channel. The at least one ON-period may be interspersed with periodic reception bursts to ensure synchronization with the other end of the communication channel.

The IPG 10 may further include a data module 602. The data module 602 may be configured to manage data relating to the identity and properties of the IPG 10. In some embodiments, the data module 602 may include one or several databases that may, for example, include information relating to the IPG 10 such as, for example, the identification of the IPG 10, one or several properties of the IPG 10, or the like. In one embodiment, the data identifying the IPG 10 may include, for example, a serial number of the IPG 10 and/or other identifiers of the

IPG 10 includes, for example, a unique identifier of the IPG 10. In some embodiments, the information associated with the property of the IPG 10 may include, for example, data identifying the function of the IPG 10, data identifying the power consumption of the IPG 10, data identifying the charge capacity of the IPG 10 and/or power storage capacity of the IPG 10, data identifying potential and/or maximum rates of charging of the IPG 10, and/or the like.

The IPG 10 may include a pulse control 604. In some embodiments, the pulse control 604 may be configured to control the generation of one or several pulses by the IPG 10. In some embodiments, for example, this may be performed based on information that identifies one or several pulse patterns, programs, or the like. This information may further specify, for example, the frequency of pulses generated by the IPG 10, the duration of pulses generated by the IPG 10, the strength and/or magnitude of pulses generated by the IPG 10, or any other details relating to the creation of one or several pulses by the IPG 10. In some embodiments, this information may specify aspects of a pulse pattern and/or pulse program, such as, for example, the duration of the pulse pattern and/or pulse program, and/or the like. In some embodiments, information relating to and/or for controlling the pulse generation of the IPG 10 may be stored within the memory.

In some embodiments, the pulse module 604 may include stimulation circuitry. The stimulation circuitry may be configured to generate and deliver one or several stimulation pulses, and specifically may be configured to generate a voltage driving a current forming one or several stimulation pulses. This circuitry may include one or several different components that may be controlled to generate the one or several stimulation pulses, to control the one or several stimulation pulses, and/or to deliver the one or several stimulation pulses.

The IPG 10 may include an energy source, such as an energy storage device 608. The energy storage device 608, which may include the energy storage features, may be any device configured to store energy and may include, for example, one or several batteries, capacitors, fuel cells, or the like.

### 4. Materials and Methods

The following description describes testing that was performed to support the feasibility and efficacy of the various disclosed embodiments of sacral nerve stimulation for the treatment of gastrointestinal dysmotility.

Pelvic nerve stimulation. The left pelvic nerve, a sub-branch of the sacral nerve specifically innervating the colorectum, was stimulated electrically using current-controlled stimulation to deliver symmetric, biphasic pulses at varying amplitudes via a suction electrode. The stimulating current was isolated (Model 2200, A-M Systems, Sequim, WA, USA) DC-filtered and monitored across a 1 kΩ resistor. A closed-loop controller was used to detect the onset and cessation of CMCs and initiate electrical stimulation. The stimulation threshold was measured as the minimum current necessary to evoke CMCs using a binary search method.

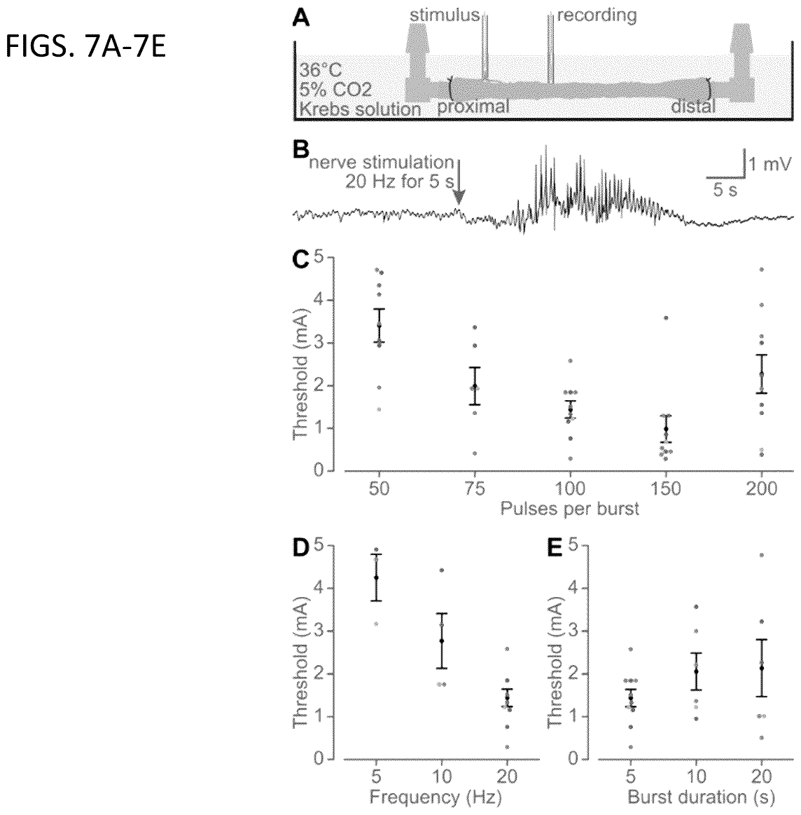

Myoelectric recording. Myoelectric activity (EMG) in the isolated mouse colon was recorded from the serosal surface opposite of the mesenteric border using one or two suction electrodes (FIG. 7).

Smooth muscle calcium imaging. Smooth muscle calcium activity was measured in acta2-RCaMP1.07 mice using a stereomicroscope (Zeiss SteREO Discovery V.20, Carl Zeiss Inc., White Plains, NY, USA).

Video analysis was carried out in MATLAB (Math Works, Natick, MA, USA). Each fluorescence video was converted to a black-and-white mask of the colon with a user-defined threshold. The diameter of the colon was approximated as the width of colon mask along the length in colon. The calcium activity was approximated as the average fluorescence intensity along the length of colon. These data were represented as colorimetric spatiotemporal maps of diameter and calcium, respectively.

The diameter spatiotemporal map was preprocessed as the first order temporal derivative of the 5-frame moving average, which represented the instantaneous change in diameter, \tilde{d}, at time i and position j (Equation 1). The calcium spatiotemporal map was preprocessed as the deviation from the mean in time normalized to the mean along the length of colon, which represented the normalized deviation, \tilde{f}, at time i and position j, where the baseline fluorescence at position j is given by f̅j (Equation 2).

$$\begin{matrix}Instantaneous change in diameter . & \\ {\tilde{d}}_{i , j} = - 1 \times ( moving mean ( {d}_{i , j} , 5 ) - moving mean ( {d}_{i - 1 , j} 5 ) ) & Equation 1.\end{matrix}$$

$$\begin{matrix}Normalized deviation of fluorescence . & \\ {\tilde{f}}_{i , j} = \frac{{f}_{i , j} - {\overline{f}}_{J}}{{\overline{f}}_{J}} & Equation 2.\end{matrix}$$

Contractile and calcium waves were identified in the instantaneous change in diameter and the normalized deviation of fluorescence, respectively, with a user-defined threshold and size exclusion. Wavefronts were identified as the leading edge of the detected waves. Wavefronts with fewer pixels than the size exclusion criteria were excluded from analysis.

Contractile and calcium waves were excluded from analysis if they were confined to the proximal or distal-most 3 cm region of the colon, to exclude cyclical activity evoked by mechanical stimulation by the barbed tubing connectors.

Computational model of colonic motility. The computational model of colonic motility consisted of a spatially distributed network of biophysically realistic point cells. Point cells were single-compartment variable-conductance models implemented using NEURON with Python, and connections between cells were constructed in NetPyNE. The cells of the network included neurons, categorized into 5 functional populations and 19 population subsets, enterochromaffin cells, enteric glial cells, 2 populations of smooth muscle fibers, and 4 populations of interstitial cells of Cajal. The parameters of the point cells and their connections were generated stochastically, unlike previous models that repeated identical units of a circuit. The transmembrane potential of each cell was defined as a function of membrane properties, membrane currents, and synaptic currents (Table 1).

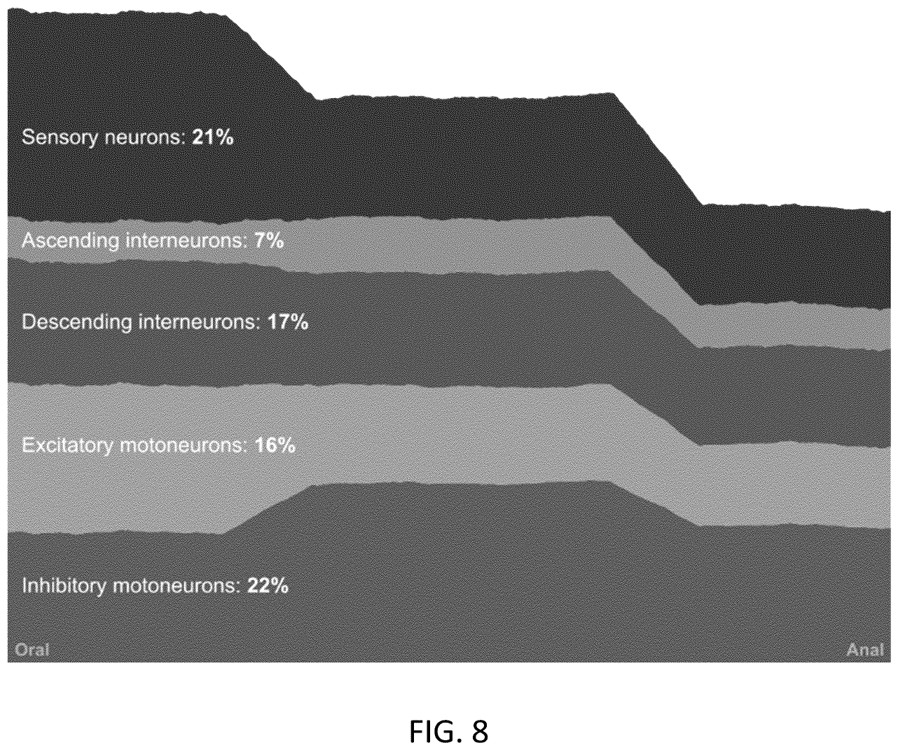

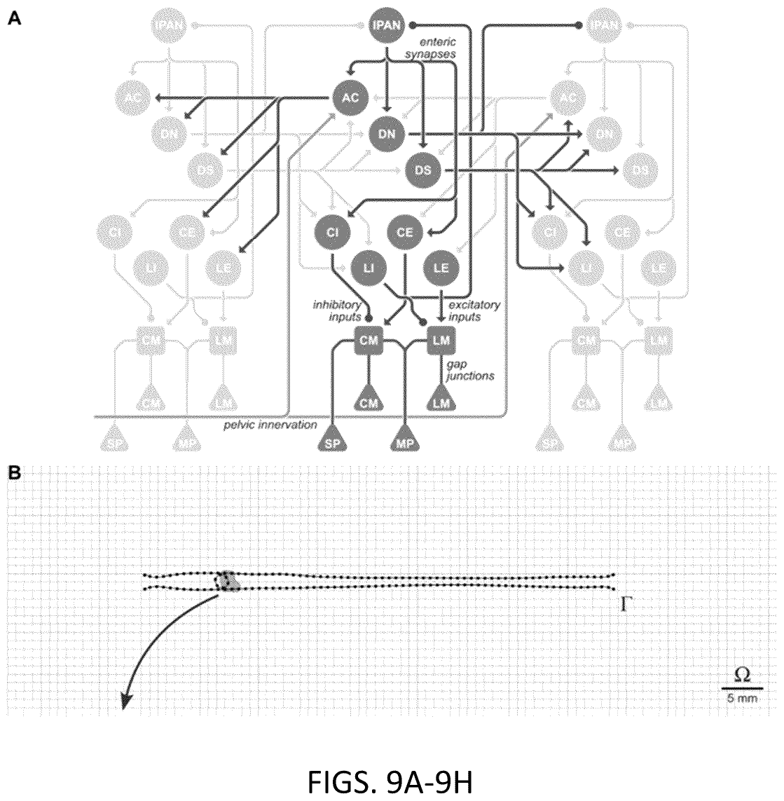

Computational models of neurons. Neurons made up 5 categories based on their functional role in the model: intrinsic primary afferent neurons (IPAN), ascending interneurons (AI), descending interneurons (DI), excitatory motor neurons (EMN), and inhibitory motor neurons (IMN). The 19 subsets, listed in Table 2, were derived from a single-cell atlas of the mouse enteric nervous system, which provided population data for each of the subsets, including abundance, prominent neurotransmitters, receptor profiles, and piezol expression. The abundance was the percentage of all neurons belonging to a given population and determined the number of cells generated for each population given a total number of neurons to model. Neurons were distributed stochastically along the length of the model based on the relative abundance in the proximal colon compared to the distal colon reported in the atlas (FIG. 8). Neuron subsets were assigned as mechanosensitive or mechano-insensitive based on Piezol expression reported in the atlas. The projection direction and distance were defined for each population based on available tracing data. Putative secretomotor-vasodilator neurons (pSVN) were excluded from the model because the model did not include mechanisms for blood flow or secretions.

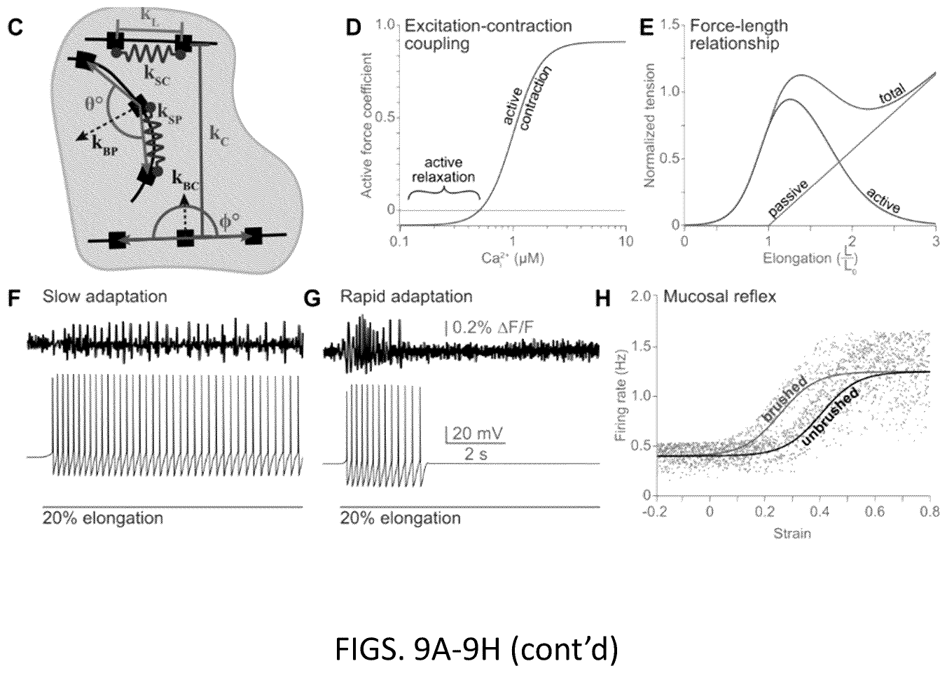

Computational models of enterochromaffin cells. Enterochromaffin cells (ECC) were modeled by independent, identical artificial spike generators evenly distributed along the colon with bistable firing rates based on distension at the position of each cell. The firing rate was defined by a sigmoidal function ranging from 0.4±0.16 Hz at baseline to 1.25±0.5 Hz during distension, and brushing the mucosa (i.e. friction between fecal pellet and colonic wall) drove the firing rate toward 1.25±0.5 Hz (FIG. 9).

Computational models of enteric glia cells. Enteric glial cells (EGC) were coupled electrically by gap junctions to adjacent glia, received synaptic input from neurons, and released slow postsynaptic inhibitory modulators to nearby cells. EGC were randomly, uniformly distributed along the colon.

Computational models of smooth muscle fibers. Smooth muscle fibers (SMF) were based on a model of colonic smooth muscle electrophysiology, and the intracellular Ca2+ concentration was translated into mechanical force based on uterine smooth muscle. SMF include circular muscle fibers (CMF) and longitudinal muscle fibers (LMF), and cells of each population were coupled electrically by gap junctions to adjacent fibers of the same population. The excitation-contraction coupling was especially important because it linked the biophysical network to the fluid dynamics components of the model; the coupling was modeled as ox in Equation 3, Equation 4, and Equation 5, where n is the Hill coefficient, Kd is the dissociation constant, and [Ca2+] i is the intracellular concentration of calcium of a given smooth muscle fiber, and the constants t1 and t2 are also defined in Table 12.

$$\begin{matrix}Excitation - contraction coupling . & \\ \frac{{δω}_{x}}{δ t} = \frac{{ω}_{x , ∞} - {ω}_{x}}{{ω}_{t}} & Equation 3\end{matrix}$$

$$\begin{matrix}Steady - state excitation - contraction coupling . & \\ {ω}_{x , ∞} = \frac{1}{1 + {\frac{{K}_{d}}{{[ {Ca}^{2 +} ]}_{i}}}^{n}} & Equation 4\end{matrix}$$

$$\begin{matrix}Excitation - contraction coupling time constant . & \\ {ω}_{t} = t 1 ( t 2 + \frac{1 - t 2}{1 + {\frac{{K}_{d}}{{[ {Ca}^{2 +} ]}_{i}}}^{n}} ) & Equation 5\end{matrix}$$

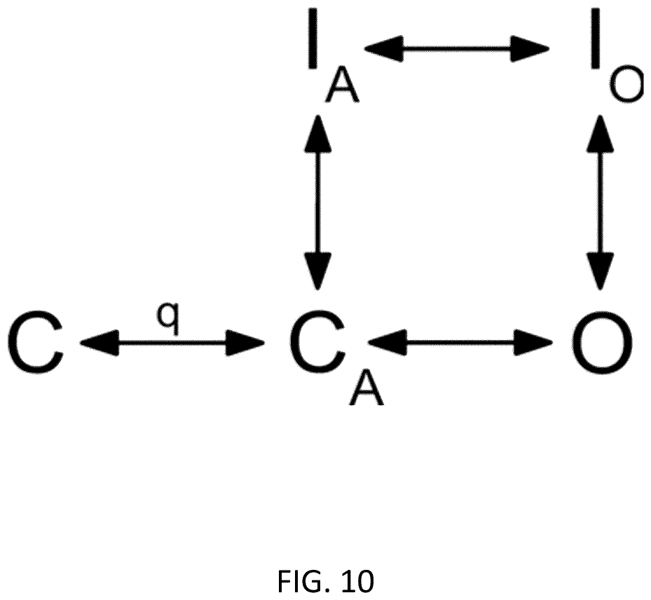

Computational models of interstitial cells of Cajal. Interstitial cells of Cajal (ICC) were based on a biophysically realistic point cell model with anoctamin 1 Ca²⁺-activated Cl⁻ channel as the pacemaking event. It was necessary to reinvent the ICC model because the Lees-Green et al. model did not match their published data. The model of ICC was adapted to include a mechanosensitive Nav1.5 channel, which was modeled as a five-state Markovian kinetic model (FIG. 10). Voltage-independent state transitions were first-order reactions with rate constant R. Voltage-dependent state transitions were three-parameter Hill equation models with maximum (b), slope (k), and EC50 (h), and minimum (c) equal to 0. The state transition from closed (C) to closed and activated (CA) was modeled with pressure sensitivity, where the slope and EC50 decreased linearly with increasing pressure amplitude, parameterized by the sensitivity to changes in pressure.

$$\begin{matrix}Hill equation . & \\ H ( x ) = \frac{b - c}{( 1 + exp ( \frac{x - h}{k} ) )} + c & Equation 6.\end{matrix}$$

The Nav1.5 model was calibrated using particle swarm optimization to replicate the activation and inactivation kinetics recorded by patch clamp electrophysiology by Beyder et al. The Nav1.5 model was calibrated in two parts: first, the pressure-independent parameters (Table 3) were calibrated to the activation and inactivation kinetics at 0 mmHg. Then, the pressure-sensitive parameters (Table 4) were calibrated to the activation and inactivation kinetics at −10, −20, −40, and −50 mmHg.

The kinetics were fit to the two-state Boltzmann model for activation and inactivation (Equation 7) where mx and mn were the minimum and maximum currents, and hB and kB were the half-point and slope, respectively. The cost function was equal to the sum of the squared errors for all activation and inactivation Boltzmann parameters (Equation 9), where \hat{θ}_{i,j}−θ_{i,j} represents the difference between the measured and target values for parameter i at pressure p. The target values for all parameters during activation and inactivation are given in (Table 5), except for mn, whose target value was 0 in all conditions.

$$\begin{matrix}Boltzmann Equation . & \\ B ( v ) = \frac{mx - mn}{( 1 + exp ( \frac{v - hB}{kB} ) )} + mn & Equation 7\end{matrix}$$

$$\begin{matrix}Nav1 \text{.5} optimization cost function . & \\ Cost = {\sum }_{\begin{matrix}p = 0 \\ - 10 \\ - 20 \\ - 40 \\ - 50\end{matrix}} {( {\sum }_{\begin{matrix}i = mx \\ mn \\ hB \\ kB\end{matrix}} {( {\hat{θ}}_{i , p} - {θ}_{i , p} )}^{2} )}_{activation} + {\sum }_{\begin{matrix}p = 0 \\ - 10 \\ - 20 \\ - 40 \\ - 50\end{matrix}} {( {\sum }_{\begin{matrix}i = mx \\ mn \\ hB \\ kB\end{matrix}} {( {\hat{θ}}_{i , p} - {θ}_{i , p} )}^{2} )}_{inactivation} & Equation 8\end{matrix}$$

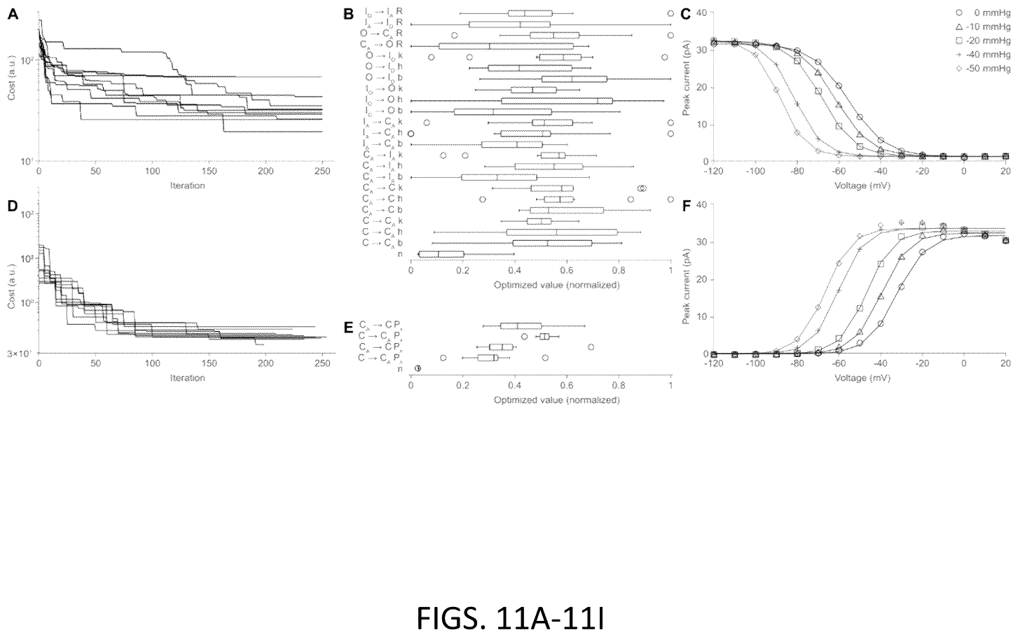

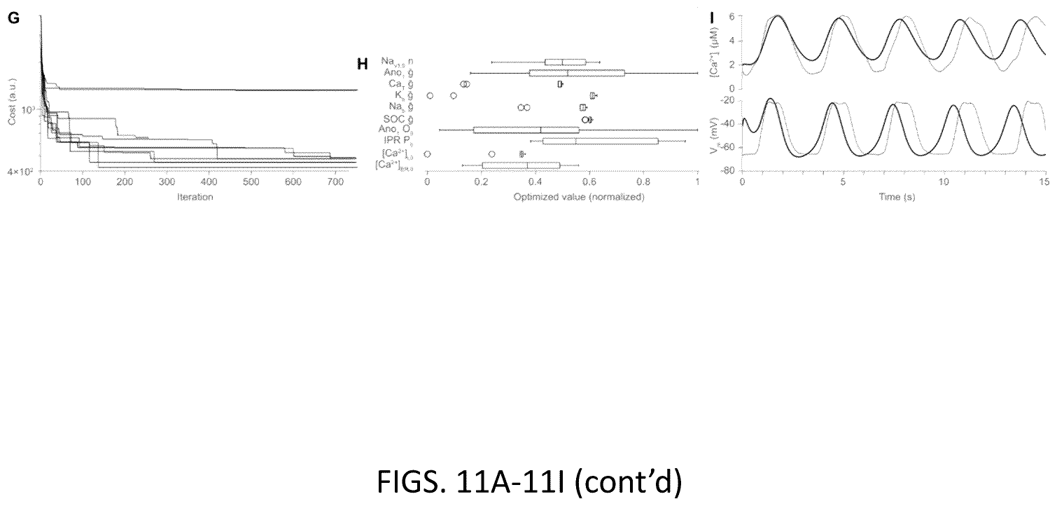

The parameters for the ICC model were determined by particle swarm optimization (FIG. 11; Table 5). The accuracy was quantified as the sum of the squares of the error of the intracellular Ca²⁺ and transmembrane potential between the original model described by Lees-Green et al., and the adapted model, where LG refers to values from the Lees-Green model. Submucosal and myenteric ICC were evenly distributed along the colon and coupled electrically by gap junctions to adjacent ICC of the same populations. Submucosal and circular intramuscular ICC were coupled electrically to adjacent CMF. Longitudinal intramuscular ICC were coupled electrically to adjacent LMF. Myenteric ICC were coupled electrically to adjacent SMF of both populations.

$$\begin{matrix}ICC model calibration . & \\ {Error}_{ICC} = {\sum }_{t = 0}^{t = 15 s} {( {V}_{{m}_{t , model}} - {V}_{{m}_{t , LG}} )}^{2} + 10 \times {\sum }_{t = 0}^{t = 15 s} {( {[ {Ca}^{2 +} ]}_{{i}_{t , model}} - {[ {Ca}^{2 +} ]}_{{i}_{t , LG}} )}^{2} & Equation 9.\end{matrix}$$

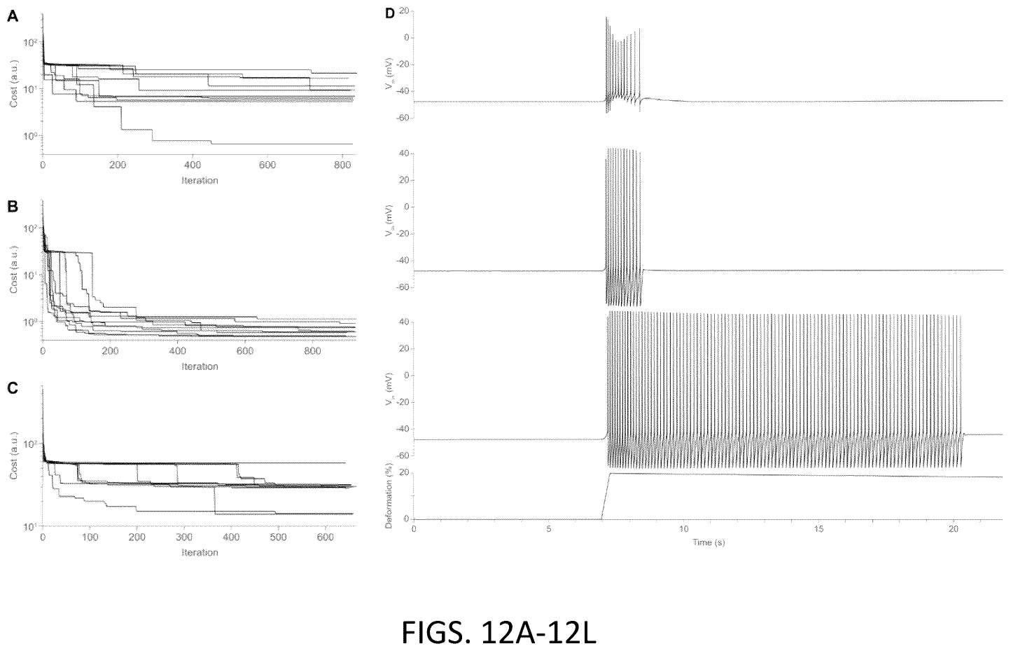

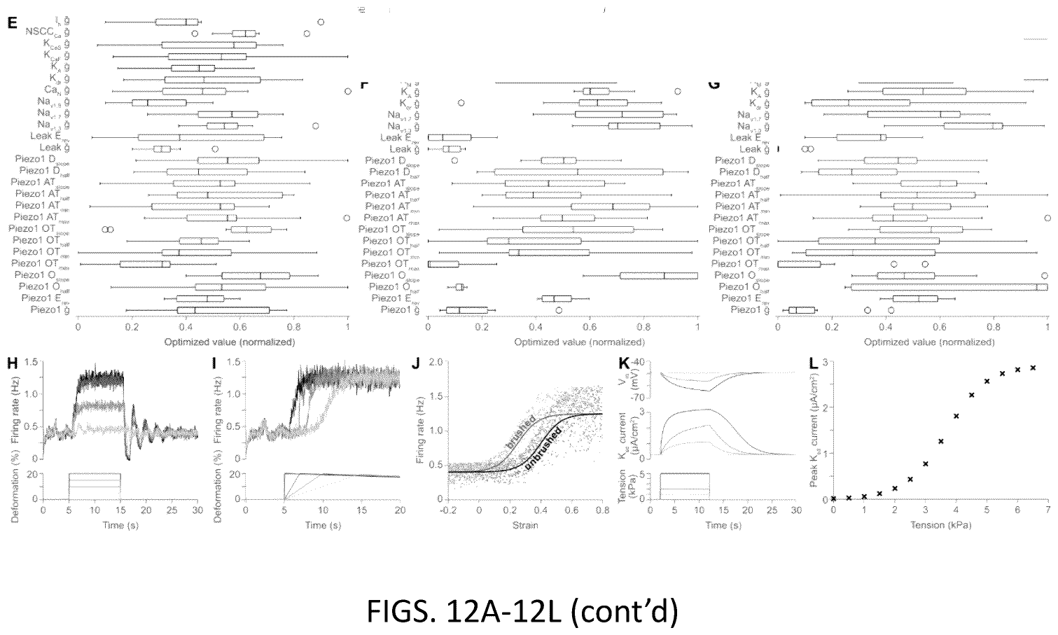

Mechanosensation. The parameters governing piezo response to tissue strain were determined by particle swarm optimization for both rapidly adapting (RAMEN) and slowly adapting (SAMEN) responses (FIG. 12). The accuracy of the ith MEN model was computed as a cost function (Equation 10), where Sx and θx refer to the actual and target values for parameter x, listed in Table 7, and QLF is the quadratic loss function between the actual, a, and target, t, values (Equation 11).

$$\begin{matrix}Cost function for mechanosensitive enteric neurons . & \\ {C}_{i} = QLF ( {S}_{b , i} , {θ}_{b , i} ) + QLF ( {S}_{d , i} , {θ}_{d , i} ) + QLF ( {S}_{s , i} , {θ}_{s , i} ) + 0.5 QLF ( {S}_{t , i} , {θ}_{t , i} ) + 0.1 QLF ( {S}_{V , i} , {θ}_{V , i} ) + 10 QLF ( {S}_{Δ , i} , {θ}_{Δ , i} ) & Equation 10\end{matrix}$$

$$\begin{matrix}Quadratic loss function . & \\ QLF ( a , t ) = {( a - t )}^{2} & Equation 11\end{matrix}$$

Network connections & mechanisms thereof. The neuronal connections in the model were determined stochastically from probability distributions for short-and long-distance projections, and gap junctions were pre-determined to connect to adjacent cells of the same population and the 3-nearest cells of a different population. The projection distance, ZA, for each neuron was drawn from a normal distribution with type-specific mean and standard deviation (Equation 12). Neurons with average projection distance less than 5 mm did not project directionally and innervated all cells of the target population with equal probability, PA→B, provided they were within the projection distance independent of direction (Equation 13). Neurons with projection distance greater than 5 mm innervated all cells of the target population with equal probability, PA→B, provided they were within the projection distance in direction of their projection (Equation 14).

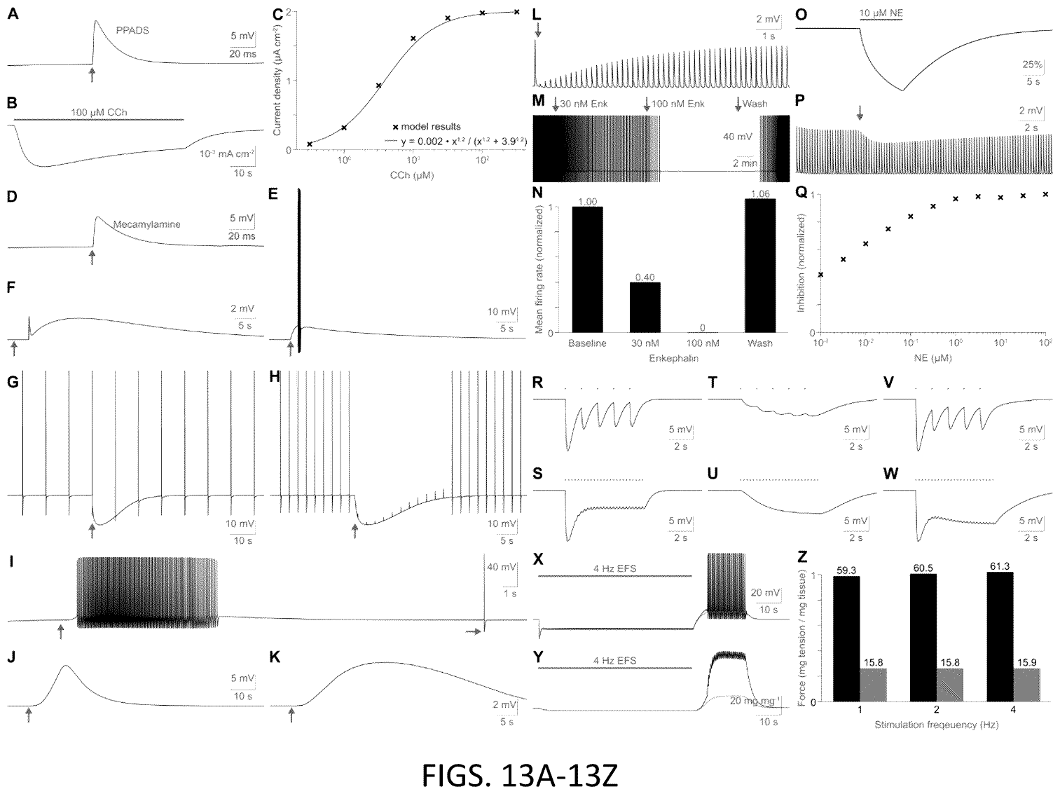

The projection probabilities, PA→B, for all synaptic connections were later optimized in the model. The mechanisms for synaptic and non-synaptic connections were biophysically defined (FIG. 13).

$$\begin{matrix}Projection distance for a cell of population A . & \\ {Z}_{A} = N ( {μ}_{A} , {σ}_{A} ) & Equation 12\end{matrix}$$

$$\begin{matrix}Short - distance projection probability . & \\ P ( A \to B ) = \{ \begin{matrix}{P}_{A \to B} & \vert {x}_{A \to B} \vert < {Z}_{A} \\ 0 & otherwise\end{matrix} & Equation 13\end{matrix}$$

$$\begin{matrix}Long - distance projection probability . & \\ P ( A \to B ) = \{ \begin{matrix}{P}_{A \to B} & 0 < {x}_{A \to B} < {Z}_{A} \\ {P}_{A \to B} & {Z}_{A} < {x}_{A \to B} < 0 \\ 0 & otherwise\end{matrix} & Equation 14\end{matrix}$$

The predominant neurotransmitter in the network was acetylcholine, acting on neurons via nicotinic receptors and the musculature via muscarinic receptors. Nicotinic transmission was modeled as a double exponential, event driven synapse. Nicotinic transmission depolarized resting postsynaptic neurons 16.4 mV with 3 ms 10-90% rise time and 9.5 ms time to half-decay. Muscarinic transmission was modeled as a four-state kinetic synapse including opened and desensitized states. It was calibrated in response to carbachol in a dose-dependent manner based on LMF from the murine small intestine.

Purinergic transmission contributed to fast excitatory postsynaptic potentials (EPSP) by acting on P2X2 receptors and was modeled as a double exponential.

Cholecystokinin was modeled by a nonspecific cation current in a double exponential, event driven synapse. Cholecystokinin depolarized resting postsynaptic neurons 13 mV, evokes one or two action potentials after 2 s, and returns to resting membrane potential after 45 s.

Serotonergic transmission was modeled as fast and slow depolarization by 5-HT3 and 5-HTIP, respectively, with double exponentials.

Somatostatin was an inhibitory neuropeptide that activated a potassium current in the postsynaptic cell, and it was modeled as a double exponential. The model for somatostatin hyperpolarized postsynaptic S-cells by 19 mV and was calibrated to intracellular recordings from cultured neurons of the rat locus coeruleus.

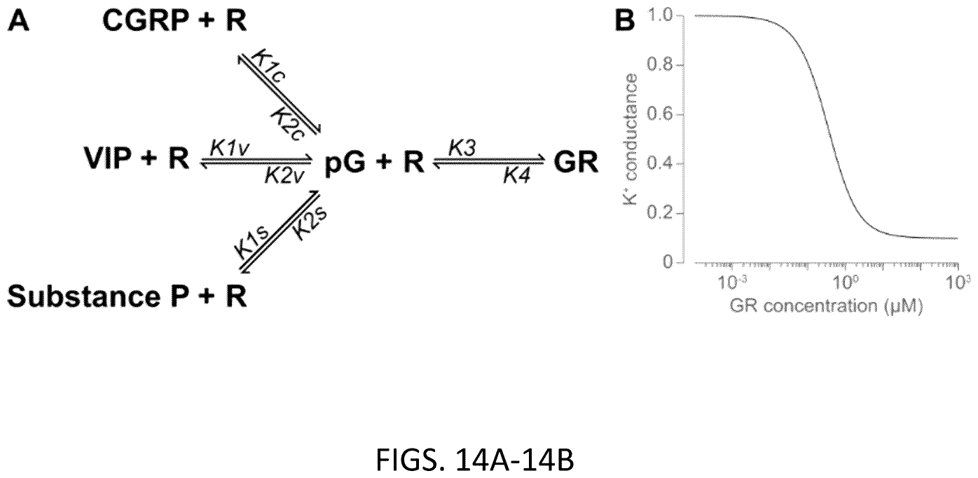

Slow excitatory postsynaptic currents were mediated by neuropeptides that reduce postsynaptic potassium conductance. This was characterized by an increase in input resistance, reversal potential equal to the potassium equilibrium potential, and reduced afterhyperpolarizing currents. Calcitonin gene-related peptide (CGRP), substance P, and vasoactive intestinal peptide (VIP) acted on the G-protein coupled-receptor pathways to reduce postsynaptic potassium conductance. In the model, each compound contributed to the rate coefficients in a three-state kinetic model (FIG. 14). The product of the kinetic model, (GR, the G-protein coupled receptor complex), reduced postsynaptic potassium conductance on a logarithmic scale in a sigmoidal relationship described by the Hill equation.

The CGRP model was based on patch clamp recordings in the isolated ileum of the guinea. In the model, CGRP evoked a train of action potentials lasting 9 s. The substance P model was based on patch clamp recordings in the isolated guinea pig myenteric plexus. In the model, substance P evoked a 6.1 mV depolarization in the postsynaptic cell 7.2 s after the synaptic event. The VIP model was based on patch clamp recordings in the guinea pig inferior mesenteric ganglion. In the model, VIP depolarized postsynaptic neurons by 5.5 mV, reaching maximum depolarization 44.6 s after the synaptic event.

Enkephalin and norepinephrine presynaptically inhibited transmitter release modeled by reducing the synaptic weight acting on the postsynaptic cell. Each mechanism involving presynaptic inhibition conveyed a blocking variable to all of the other postsynaptic mechanisms present in the postsynaptic cell, thus reducing the synaptic weight of incoming neurotransmission.

Enkephalin inhibited presynaptic release and hyperpolarized the postsynaptic cells through increased potassium channel conductance. The receptor was modeled as a four-state kinetic system: unbound, bound inactive, opened channel, and blocking states. The blocking state was referenced by other synaptic point process mechanisms present in the postsynaptic cell and decreased their conductivity accordingly. The open channel state determined the conductivity of a potassium current. The time constant and amplitude of the potassium current were based on opioid inhibition of neurons encoding the respiratory rate.

Norepinephrine acted through presynaptic inhibition. The timing constraints of the response were modeled based on analysis conducted in HEK-293 cells transfected with the α2A-adrenergic receptor.

Inhibitory junction potentials (IJP) included fast and slow components via purinergic and nitrergic transmission, respectively. Each component was modeled as double exponentials with inactivation and reactivation. The time constants and amplitude were calibrated to evoked IJP in the colon with a selective purinergic receptor antagonist and a nitric oxide synthase inhibitor.

Excitatory junction potentials (EJP) were mediated through acetylcholine acting on muscarinic receptors and included substance P. The amplitude of muscular response to muscarinic agonists and substance P were calibrated to the contractile force evoked by electrical field stimulation of the pyloric circular muscle excised from rats with and without atropine, a muscarinic antagonist.

Gap junctions between EGC, SMF, and ICC are mostly connexin-43 bidirectional hemichannels and modeled as such.

Immersed boundary model & fluid dynamics. The oral and anal end of the colon were open and fixed in the Eulerian grid, Q (FIG. 9B). On both upper and lower boundaries, adjacent points were tethered to one another with Hookcan springs, torsional springs, and longitudinal muscle fibers. Points opposite from one another on either boundary were tethered by circular muscle fibers. The parameters for the immersed boundary implementation are given in Table 18.

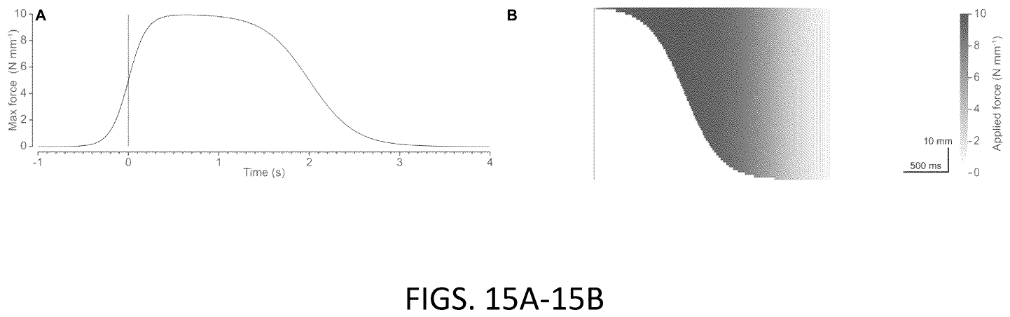

Fluid distension was modeled as an external forcing function applied to fluid in the Eulerian domain. The forcing function was applied on the central axis of the colon as a wave expanding for 2 s from proximal to distal ends (FIG. 15).

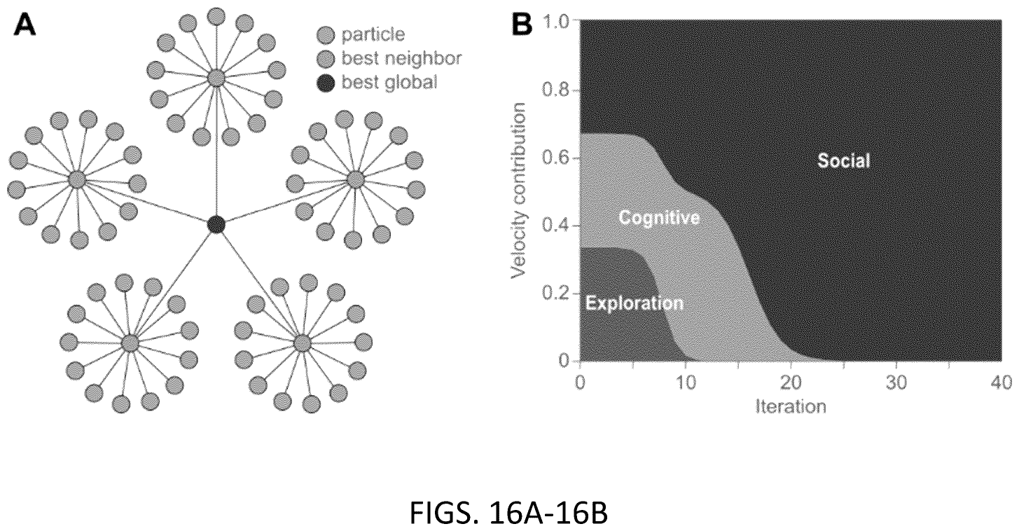

Particle swarm optimization. The model parameters for the maximum conductance of gap junctions and synapses, connection probability between populations, and Hookcan and torsional spring constants were determined by particle swarm optimization (PSO) using a modified Star topology. The swarm was divided into neighborhoods in which all particles in the neighborhood were connected with one another (FIG. 16). The PSO included inertia weight, w, and a random walk term to combat stagnation. The velocity of parameter j for particle i at time t+1 is given by Equation 15.

$$\begin{matrix}Particle velocity equation . & \\ {V}_{t + 1 , j}^{( i )} = {wV}_{t , j}^{( i )} + \frac{{S}_{t} ( {G}_{t , j}^{( i )} - {X}_{t , j}^{( i )} ) + {C}_{t} ( {P}_{t , j}^{( i )} - {X}_{t , j}^{( i )} ) + {R}_{t} {v}_{max} ( {U}_{j} - {L}_{j} )}{{S}_{t} + {C}_{t} + {R}_{t}} & Equation 15\end{matrix}$$

The previous value of parameter j is given by X(i)t,j, and the neighborhood and global best particle values for parameter j are given by P(i)t,j and G(i)t,j, respectively, across all generations. Uj and Lj were the upper and lower bounds of parameter j, respectively. Coefficients St, Ct, and Rt represented the social, cognitive, random walk (or exploration), respectively, and were determined as a function of the iteration (Equation 16, Equation 17, and Equation 18).

$$\begin{matrix}Social coefficient . & \\ {S}_{t} = 1 & Equation 16\end{matrix}$$

$$\begin{matrix}Cognitive coefficient . & \\ {C}_{t} = \frac{1}{1 + {e}^{\frac{20 t}{d} - 10}} & Equation 17\end{matrix}$$

$$\begin{matrix}Exploration coefficient . & \\ {R}_{t} = \frac{1}{1 + {e}^{\frac{t}{d} - 10}} & Equation 18\end{matrix}$$

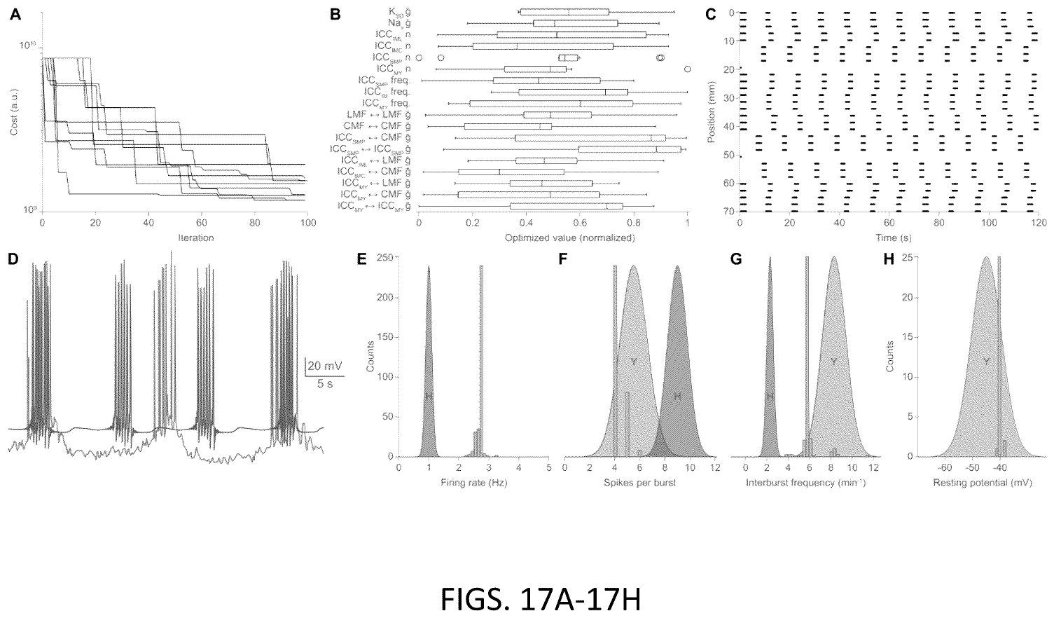

Calibrating model parameters to myogenic activity. Myogenic activity in the model was designed to replicate bursting activity recorded intracellularly in the isolated mouse colon. The PSO (Table 9) was used to calibrate the maximum conductance of gap junctions (indicated with ↔between populations) and mechanosensitive ion channels, pacemaker frequencies, and number of pacemaker cells within the electrical syncytium (FIG. 17; Table 10).

The upper and lower bounds (constraints) for each parameter were defined as the target mean±5 standard deviations. The distributions of values were compared from the model to the distribution for each parameter. The distribution bins were constant across all parameters as 50 bins between the upper and lower bounds. In the cost function, RMSE refers to the root mean square error between the observed and the predicted (caret) distributions.

Equation 19 was determined by the target resting membrane potential, v, bursts per minute, r, spikes per burst, k, spikes per minute, f, and bursting duration, d, of smooth muscle fibers (Table 11) as reported previously. The upper and lower bounds (constraints) for each parameter were defined as the target mean±5 standard deviations. The distributions of values were compared from the model to the distribution for each parameter. The distribution bins were constant across all parameters as 50 bins between the upper and lower bounds. In the cost function, RMSE refers to the root mean square error between the observed and the predicted (caret) distributions.

$$\begin{matrix}Myogenic PSO cost function . & \\ {Cost}_{myogenic} = {\sum }_{i = 1}^{s} ( {\sum }_{m = CM , LM} ( 0.1 \times RMSE ( {v}_{m} , {\hat{v}}_{m} ) + RMSE ( {r}_{m} , {\hat{r}}_{m} ) + RMSE ( {k}_{m} , {\hat{k}}_{m} ) + RMSE ( {f}_{m} , {\hat{f}}_{m} ) ) + RMSE ( d , \hat{d} ) ) & Equation 19\end{matrix}$$

Fluid distension. The probability of innervation and synaptic weights were estimated by replicating the diameter and calcium spatiotemporal responses to fluid distension. The precise parameters estimated in this optimization are given in Table 12 and Table 13.

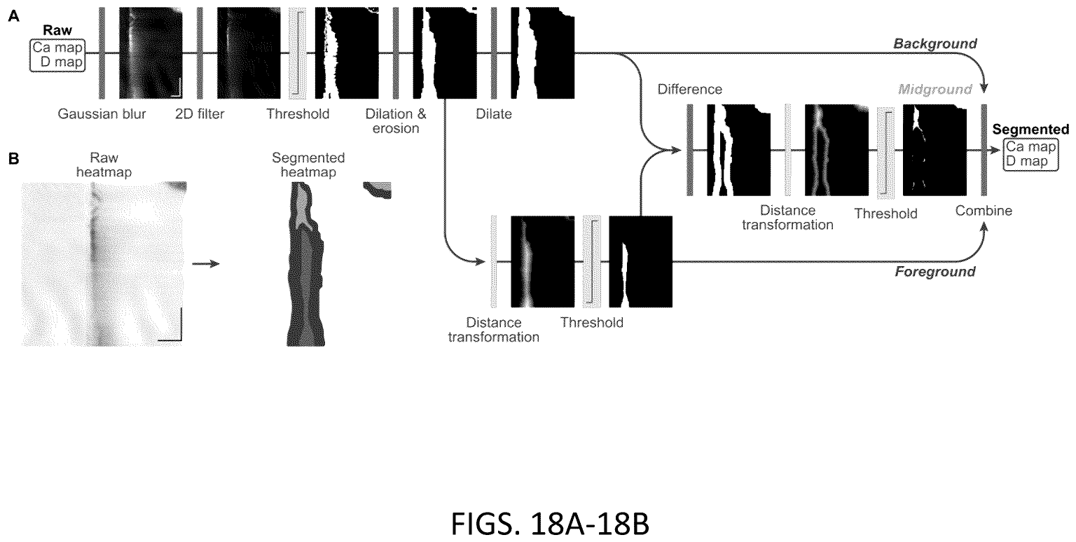

Cost function preprocessing. The cost function used to optimize the parameters of the model response to fluid distension is described in Equation 20, where M represents the root mean square error, calculated pixel-by-pixel, between the mouse and model for a given spatiotemporal map: calcium fluorescence (Ca) and diameter (D). The process for comparing the spatiotemporal maps is shown in FIG. 18, in which the spatiotemporal maps for diameter and calcium fluorescence from the computational and animal models were segmented identically.

$$\begin{matrix}Cost function for spatiotemporal maps . & \\ Cost = 2 \times {M}_{Ca} + {M}_{D} & Equation 20\end{matrix}$$

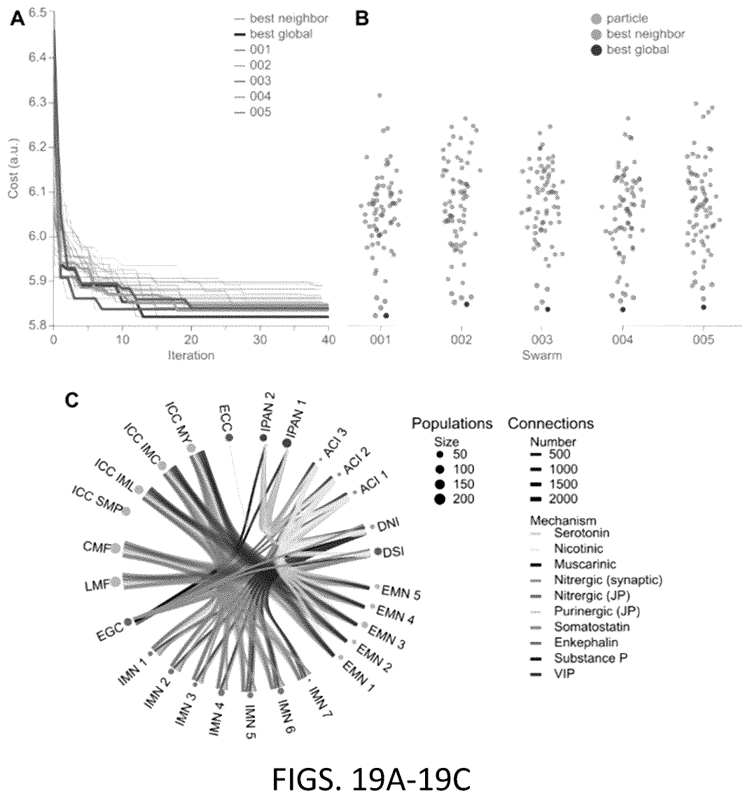

Calibrating model parameters to Ca2+ waves evoked by fluid distension. The particle swarm optimization continued until the termination criteria were met (Equation 21), where Q0.25 is the 25th percentile, p is the cost for each particle, and g15-are costs for the global-best particles in the last 15 iterations. The 5 independent swarms converged to the same cost (5.83 [5.82, 5.85]), and the coefficient of variation for the final performance across all particles for each swarm was 0.014 [0.012, 0.015] (FIG. 19).

$$\begin{matrix}Termination criteria . & \\ Terminate if ... \{ \begin{matrix}{Q}_{0.25} ( iteration ) \geq 41 \\ or \\ \frac{IQR ( p )}{median ( p )} \leq 0.005 and \frac{IQR ( {g}_{15 -} )}{median ( {g}_{15 -} )} \leq 0.001\end{matrix} & Equation 21\end{matrix}$$

Pellet propagation performance. The mechanical parameters of the immersed boundary model were tuned to reproduce the pellet trajectory in the isolated mouse colon (Table 14). The average velocity of 1-1.5-, and 2-mm-diameter fecal pellets in the isolated mouse colon was 0.29±0.10, 0.40±0.08, and 0.55±0.07 mm/s, respectively. The PSO used 50 particles, decaying maximum velocity, vmax′ (Equation 22), and decaying inertia weight, w′ (Equation 23). Each particle comprised nine simulations to examine three different pellet diameters and three different random seeds. The total cost of a particle for a given iteration was the sum of all nine simulations. Particles were updated after all nine simulations were completed, and new simulations for a given particle were not dependent on the completion status of simulations of other particles.

$$\begin{matrix}Maximum velocity decaying with iteration . & \\ {{v}_{max}}^{′} ( i ) = max \{ \begin{matrix}{v}_{max} \times {e}^{- 2 \times \frac{i}{d}} \\ 0.1 \times {v}_{max}\end{matrix} & Equation 22\end{matrix}$$

$$\begin{matrix}Inertia weight decaying with iteration . & \\ {w}^{′} ( i ) = \frac{w \times d}{{( i - 2 \times d )}^{2}} & Equation 23\end{matrix}$$

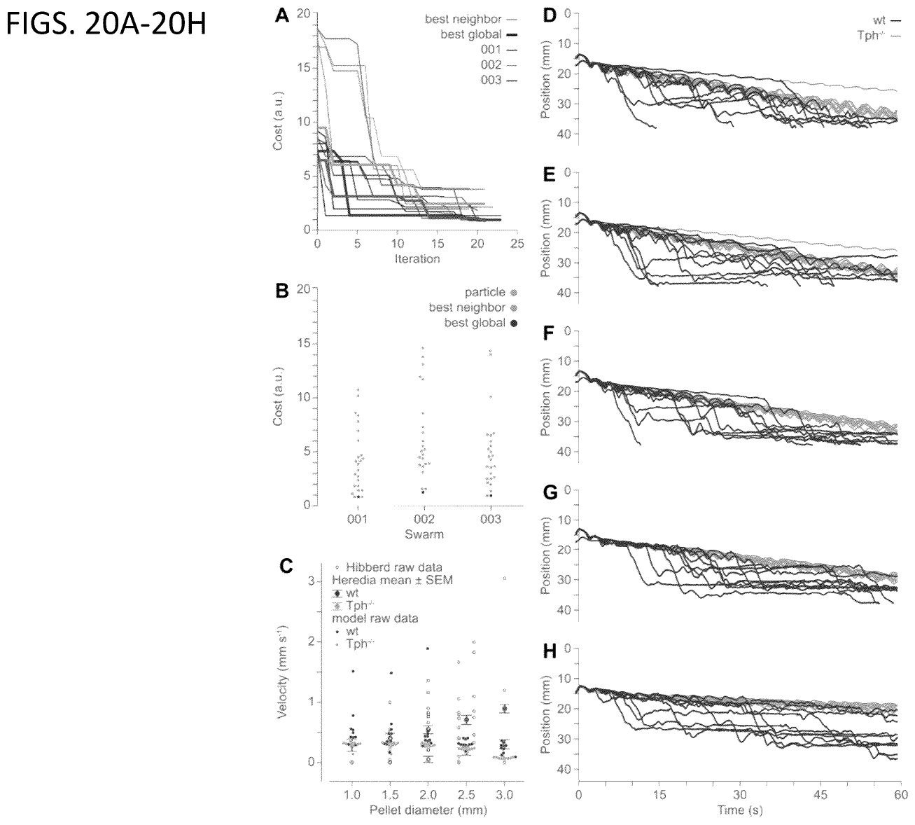

The best performing particle across all swarms recreated average velocity of 1-1.5-, and 2-mm-diameter fecal pellets equal to 0.33±0.08, 0.40±0.04, and 0.46±0.03 mm/s, respectively. The stochasticity within the model created a distribution of pellet velocity for each random instantiation of the model. The model performed better for small diameter pellets (1-2 mm diameter) than it did for large diameter pellets (2.5-3 mm diameter) (FIG. 20; Table 15)

Pressure & pelvic nerve stimulation. Pelvic nerve stimulation was simulated as synaptic inputs to populations of neurons in the biophysical network. The probability of innervation and the synaptic weight were estimated for each postsynaptic population (Table 16). The weight of the pelvic synaptic inputs was modeled as a probability distribution as a function of position of the ith ascending or excitatory motor neuron, xi (Equation 24), where wpelvic is the synaptic weight, and xpelvic and spelvic are the center and the half-span of pelvic innervation, respectively. The position and shape of the innervation probability and the synaptic weight were chosen as the best performing solutions from a Monte Carlo simulation with all parameters varied concurrently and sampled with the Latin hypercube method. The model performance was scored as the sum across five random instantiations of the model, with each score defined as the difference in the rectal pressure trace between the model and the rat.

$$\begin{matrix}Pelvic innervation probability . & \\ {{w}_{pelvic}}^{′} ( {x}_{i} ) = \{ \begin{matrix}{w}_{pelvic} & {x}_{i} - {x}_{pelvic} < {s}_{pelvic} \\ 0 & otherwise\end{matrix} & Equation 24\end{matrix}$$

Colonic emptying outcomes from the computational model. The average velocity for all four pellets were measured in colonic emptying experiments and the average velocity across all four pellets were reported. The pellet trajectory was extrapolated to estimate fecal pellet output in the model. A linear regression was performed on the position of each pellet in time and calculated the average velocity as the slope of the linear regression.

Pivotal clinical trials to treat constipation with prucalopride were limited to patients with two or fewer spontaneous complete bowel movements (SCBM) per week. Each of the three pivotal double-blind, placebo-controlled, 12-week studies defined a clinically relevant improvement as an increase of 1 SCBM per week to at least 3 SCBM per week. Therefore, positive responders to SNS treatment were defined as 50% improvement in average pellet velocity and 50% improvement in fecal pellet output compared to the mean reported in the slow transit model.

The primary tunable parameter of conventional, tonic stimulation is pulse repetition frequency because pulse amplitude and pulse width interchangeably affect the propensity for stimulation to activate a given fiber within a nerve, which are limited by patient sensation and tolerance. Burst-patterned stimulation introduces two additional temporal parameters: burst duration and interburst interval.

Cuff electrode placement and rectal pressure recording in the anesthetized rat. Rats were positioned prone under urethane anesthesia. The gluteus maximus and tensor fascia lata were separated by blunt dissection and the greater sciatic foramen was identified by tracing proximally the sciatic nerve. The L6-S1 nerve trunk was identified deeper and medial to the sciatic nerve, and a 300-μm nerve cuff was placed around the L6-S1 nerve trunk. The cuff placement was confirmed by performing a bilateral sacral laminectomy. The L5, L6, S1, and S2 nerve trunks were exposed on both sides. After confirming the L6-S1 nerve cuff placement, 200-μm nerve cuffs were placed on the medial SI and lateral SI branches. After the rats were anesthetized, colonic and rectal pressure were recorded using 3-cm-long, abdominal pressure balloons. The balloons were deflated, lubricated, and inserted into the rectum in series, occupying the distal-most 6 cm of the colon and rectum. Branches of the lumbosacral nerve roots were mapped to evoked pressure responses in the colon and to evoked EMG responses in the base of the tail.

Computational model of colonic motility. The computational model of colonic motility was built in two parts: a biophysical network model and an immersed boundary model of fluid dynamics. The biophysical network conveyed contractile forces from smooth muscle fibers to the immersed boundary model, and the immersed boundary model conveyed tension, strain, and mucosal reflexes back to the biophysical network.

Biophysical network. The biophysical network included connected biophysical models of enterochromaffin cells (ECC), enteric glial cells (EGC), smooth muscle fibers (SMF), and interstitial cells of Cajal (ICC), which were linearly distributed along the colon and connected to adjacent cells by gap junctions, and enteric neurons (FIG. 9A). Enteric neurons were distributed in the model based on their prevalence along the length of colon in the mouse (FIG. 8), and their connections were generated stochastically based on optimized parameter estimation.

Fluid dynamics. The colon was modeled as a 2-dimensional tube with deformable walls, and the fluid dynamics were solved numerically with a 2-dimensional immersed boundary method. The colon was represented by a cylindrically symmetric set of points, X, in a Lagrangian field, Γ, immersed in a fluid in a fixed Cartesian grid in the Eulerian domain, Ω (FIGS. 9B-9C).

Muscle contractions. Muscle contraction models were based on the excitation-contraction coupling recorded from uterine smooth muscle. Cytosolic Ca2+ determined the percent activation for active contractions (FIG. 9D). Active relaxation in smooth muscle was modeled with a negative offset in active force coefficient to simulate the stiffness of collagen and the extracellular matrix. The force generated by muscle contractions was the sum of active and passive contractile force as a function of the normalized fiber length (FIG. 9E).

Mechanosensation. Mechanosensation was encoded in the model among ascending and descending excitatory interneurons, ECC, and SMF (FIG. 12). Mechanosensitive enteric neurons (MEN) were categorized as rapidly adapting and slowly adapting (FIGS. 12F-12G) based on characterization by intraganglionic fluid injections. The model conferred mechanosensation to neurons with a piezo current. Non-neuronal mechanosensation included ECC (FIG. 10H and FIGS. 12H-12J) and SMF. Myogenic accommodation to distension in SMF (FIGS. 12K-12L) was encoded in the model by stretch-dependent potassium channels.

Mucosal reflex. The mucosal reflex was encoded by spontaneous release of serotonin from ECC. The spontaneous release was modeled as Poisson events, and the interval between events decreased with increasing mechanical strain. The mucosal response to distortion, such as brushing the mucosa or the presence of intraluminal content, was modeled by decreasing the interval of Poisson events. Brushing the mucosa reduced the ECC onset threshold by shifting the response curve to lower mechanical strains (FIG. 9H).

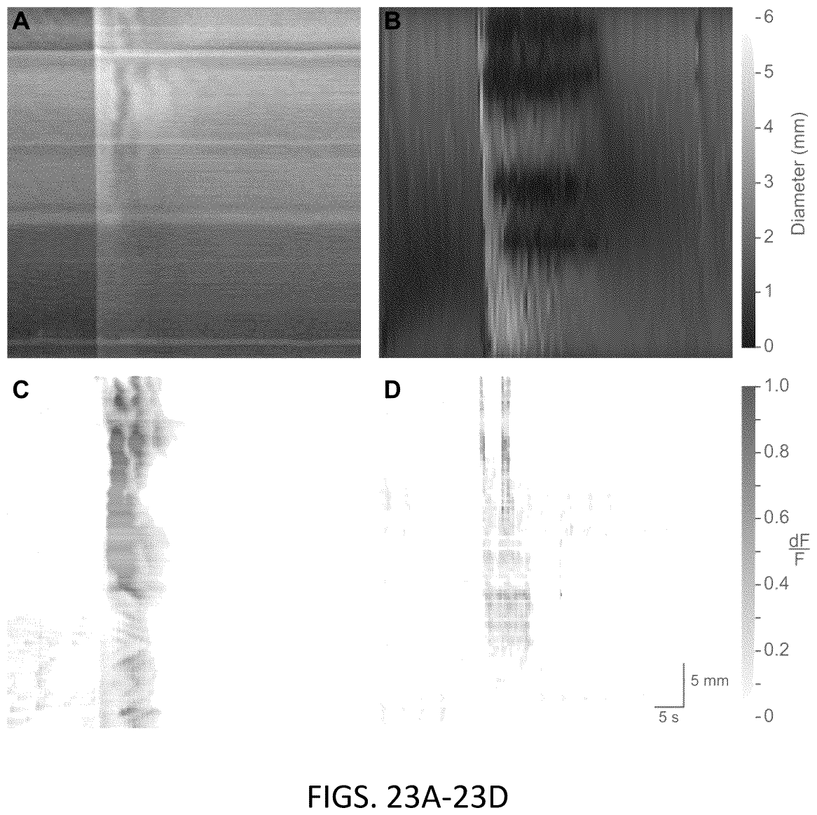

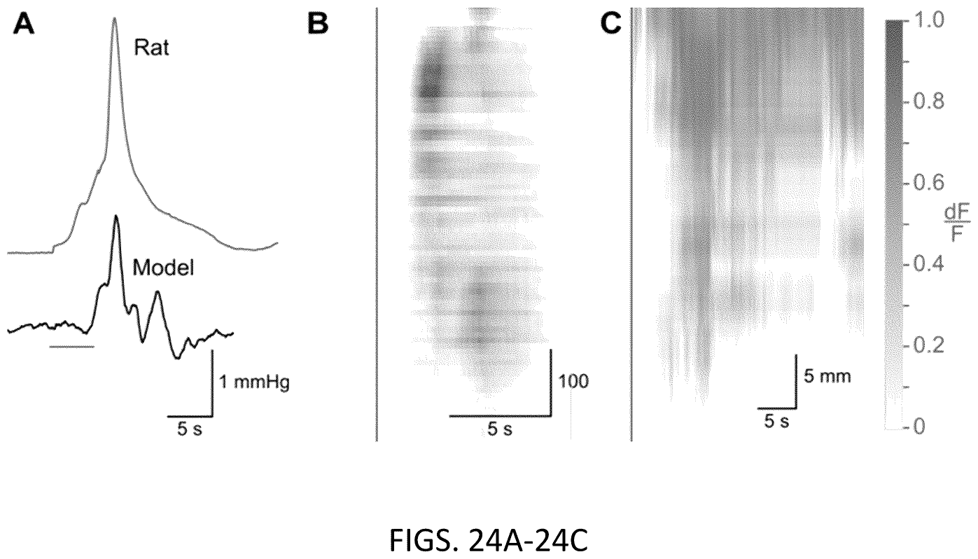

Model validation. The response to fluid distension and pellet propagation were replicated in the computational model of colonic motility. Connectivity parameter values were estimated using particle swarm optimization (PSO; FIG. 23), and the optimized model reproduced spatiotemporal heatmaps of colon diameter and calcium-associated fluorescence measured in the isolated mouse colon (FIG. 16).

The value of mechanical parameters was then estimated to replicate the average pellet velocity. The model closely replicated the average velocity of 1, 1.5, and 2-mm-diameter pellets, but not 2.5 or 3-mm-diameter pellets (FIG. 20). The diameter of naturally formed pellets in the mouse colon is approximately 2 mm (Costa et al., 2021, Heredia et al., 2010, Heredia et al., 2013, Tan et al., 2020). The velocity of 2-mm-diameter pellets in the model was 0.58±0.17 mm s¹, compared to values reported in the literature: 0.67±0.11, 0.55±0.19, and 0.39±0.03 mm s⁻¹.

Sacral nerve stimulation in the computational model of colonic motility. The mean pressure response to L6-S1 nerve stimulation in the urethane-anesthetized rat was used to estimate sacral nerve innervation parameters in the computational model of colonic motility. The mean pressure response to L6-S1 nerve stimulation was calculated in the rat as the average of all responses that reached or exceeded 5 mmHg, and the innervation parameters were estimated in the computational model to replicate the shape of the pressure trace from the rat (FIG. 24A; Table 16). The fluorescent calcium response to pelvic nerve stimulation in the isolated mouse colon was reproduced by the computational model (FIGS. 24B-24C). The rat colon and computational model reached peak pressure 6.9 s and 7.4 s after stimulus onset, respectively. The number of pulses delivered at 20 Hz did not correlate with the peak pressure response in the model (Table 21) or the mean EMG response in the isolated mouse colon (Table 22).

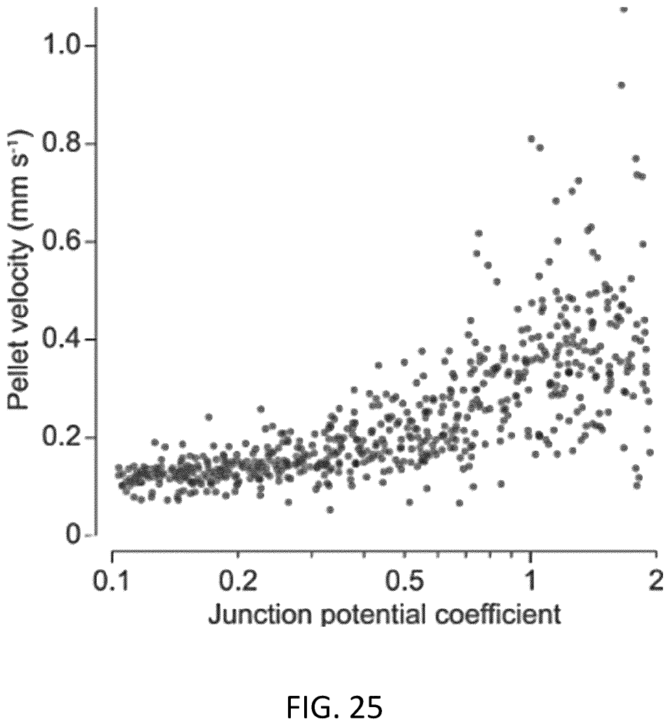

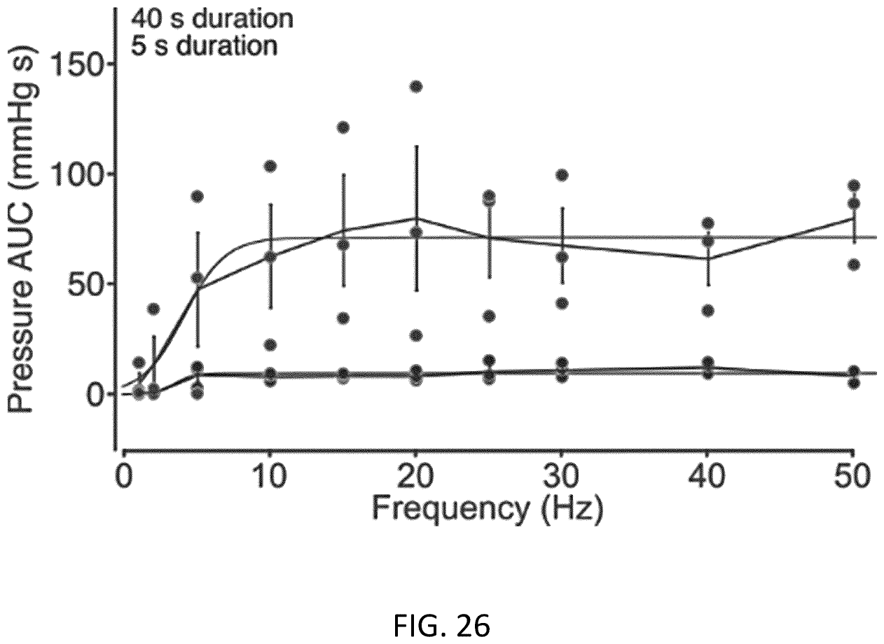

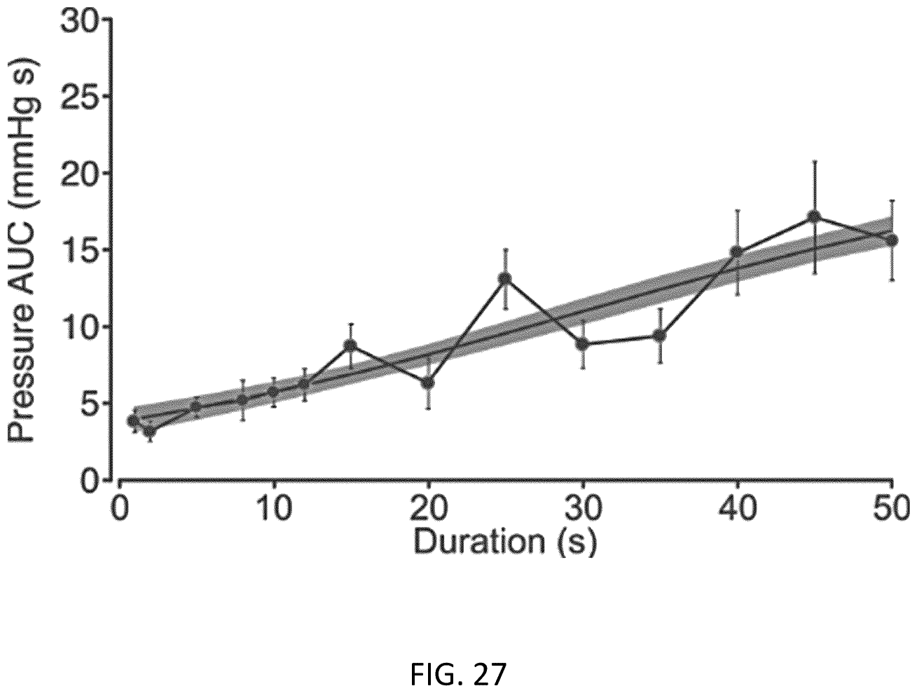

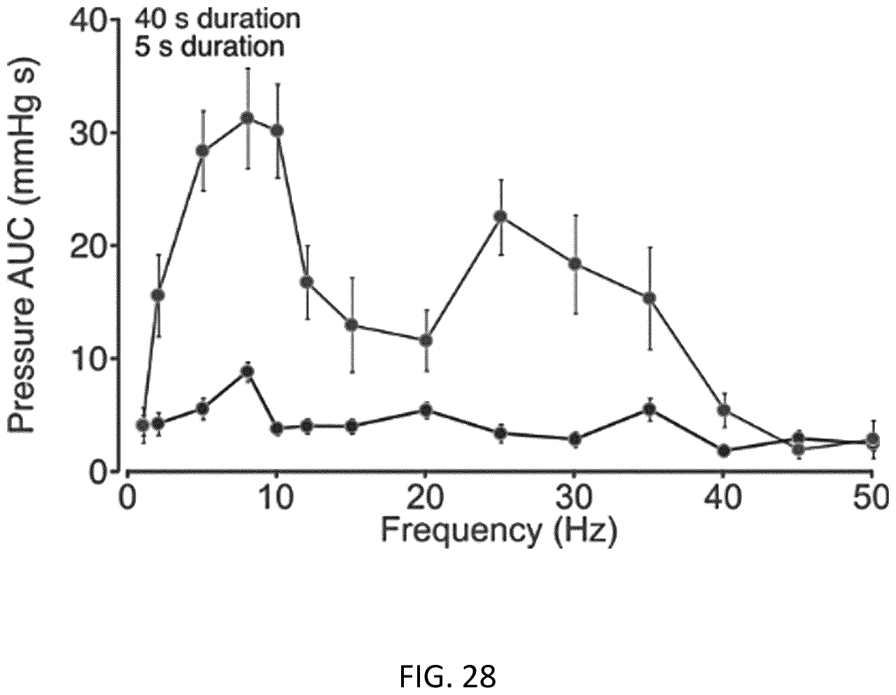

Modeling delayed colonic emptying. Colonic motility was modeled as the propulsion of four pellets in the colon. Two metrics were used to quantify motility: average pellet velocity and fecal pellet output extrapolated from the pellet trajectory. The normal transit model closely matched the fecal pellet output reported in freely behaving mice (1.3±0.3 pellets per hour, n=6). Delayed colonic transit was simulated by reducing the strength of neuromuscular junctions (FIG. 25) The model of slow transit decreased the average pellet velocity from 0.42±0.19 to 0.10±0.05 mm s−1 and decreased the fecal pellet output from 1.38±0.53 to 0.5±0.23 hr−1 compared to the model of normal transit.

### 5. EXAMPLES

As described further herein, disrupted communication along the brain-gut axis contributes to impaired visceral function and debilitating symptoms (e.g., gastrointestinal dysmotility disorders). Colonic dysmotility in particular remains poorly managed by conventional pharmaceuticals. One objective of the present disclosure was to restore proper gastrointestinal motility by electrical stimulation of the sacral nerves, optimize stimulation patterns to relieve one or more symptoms of gastrointestinal dysmotility (e.g., constipation), and elucidate the mechanisms of motor patterns evoked by stimulation. Through combinations of computational, ex vivo, and anesthetized and awake in vivo models, propulsive, prokinetic motility was evoked by burst-patterned sacral nerve stimulation and constipation was relieved in awake, behaving rats. Further, various stimulation parameters were systematically varied, including stimulation frequency, stimulation duration, and interburst interval, and minimum effective parameters were determined to maximize anorectal contractions. As described further below, results of the present disclosure identified precise temporal patterns of sacral nerve stimulation that relieved constipation in rats.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Pelvic nerve stimulation in the isolated mouse colon. Previous work led to the consideration of alternatives to continuous, tonic stimulation used in clinical sacral nerve stimulation. Electrical stimulation of the colon directly temporarily halts colonic motor complexes, and after each colonic motor complex there exists a refractory period before a subsequent motor complex can occur. It was hypothesized that intermittent bursts of electrical stimulation would more efficiently evoke rhythmic, propulsive contractions than tonic stimulation. Thus, experiments were conducted to obtain direct evidence for the effects of stimulation frequency, burst duration, and interburst interval on colonic motility. Temporal patterns of electrical stimulation were systematically optimized for sacral nerve stimulation, which led to the identification of temporal patterns that were more effective than clinical sacral nerve stimulation in the loperamide model of slow-transit constipation in awake, behaving rats.

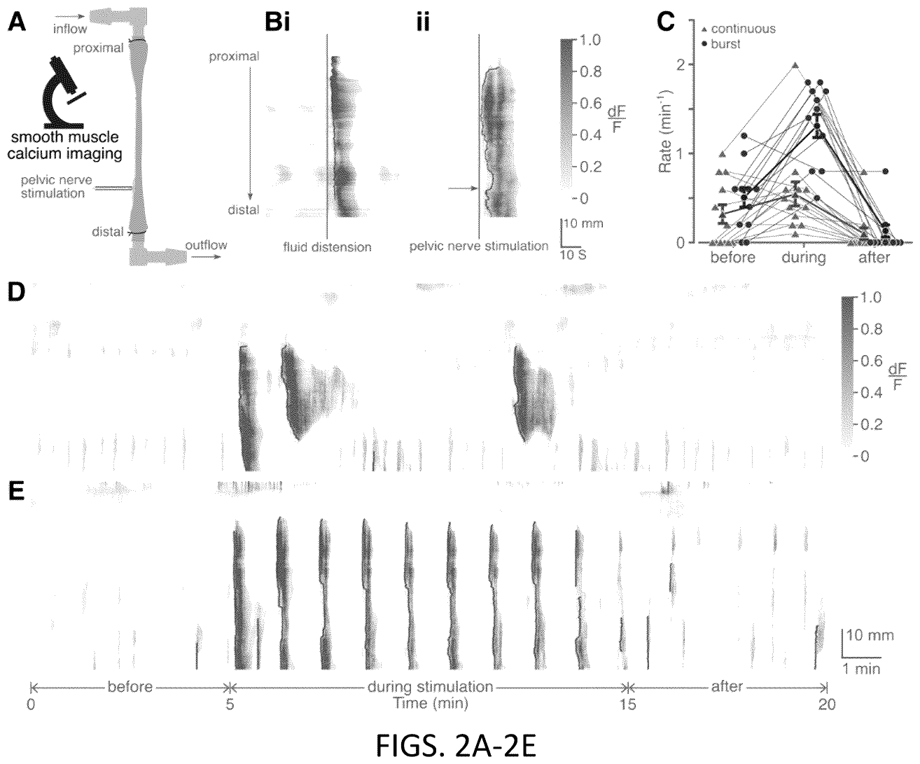

Temporal patterns of nerve stimulation in the isolated colon were compared from acta2-RCaMP1.07 transgenic mice. Smooth muscle calcium imaging revealed spatiotemporal patterns of propagating contractions (FIG. 1A). Calcium waves indicative of propagating contractions were evoked similarly by fluid distension (FIG. 2Bi) and by electrical stimulation of the pelvic nerve (FIG. 2Bii).

The rate of calcium waves was compared before, during, and after electrical stimulation of the pelvic nerve (FIGS. 2E-2C). Tonic 14 Hz stimulation evoked contractions promptly at the onset of stimulation and less frequently thereafter (FIG. 2D). In contrast, burst stimulation, consisting of 20 Hz, 5 s bursts with 60 s between bursts, evoked contractions at the onset of stimulation and continued to evoke contractions with subsequent bursts (FIG. 2E). Burst-patterned pelvic nerve stimulation increased the rate of calcium waves more than tonic stimulation (0.85±0.58 vs. 0.24±0.44 min⁻¹, n≥11, t ratio=2.9, p<0.01).

### Example 2

Exhaustive parameter search in a computational model of colonic motility. Experiments were conducted to determine the most propulsive parameters for burst-patterned sacral nerve stimulation. However, examining parameters individually and empirically is impractical due to the large, three-dimensional parameter space and fails to capture non-linear interactions between parameters. Therefore, a computational model was built and validated to evaluate efficiently and systematically parameters of stimulation and the interactions thereof.

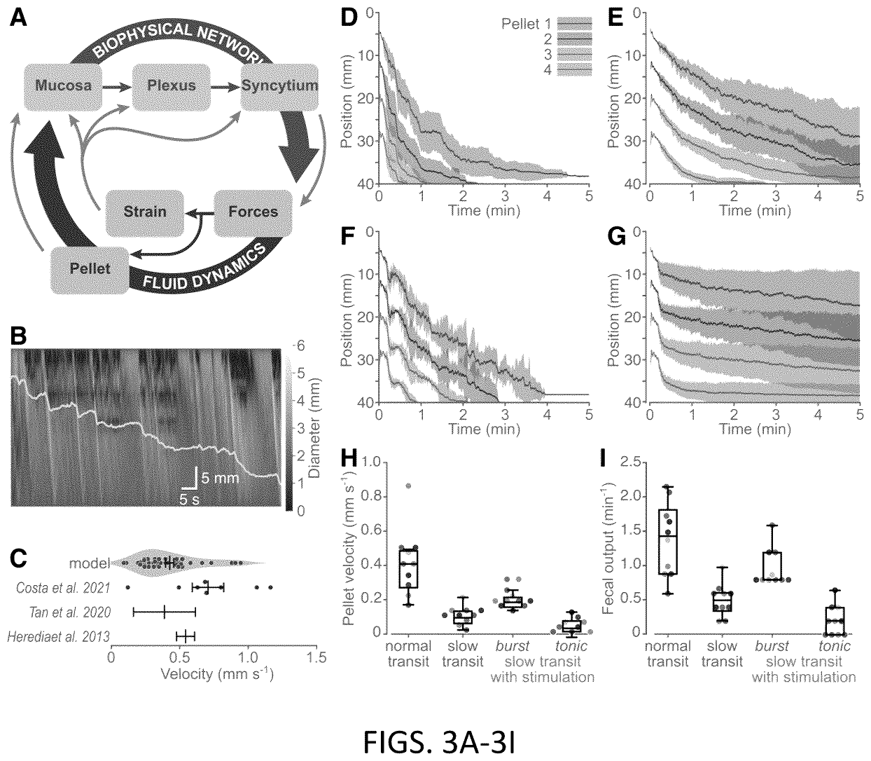

A computational model of colonic motility incorporating a biophysical network of cells with fluid dynamics was built to simulate peristalsis in the colon (FIG. 3A). The model simulated propagation of a pellet through the colon (FIG. 3B), and the average pellet velocity was consistent with the reported average velocity in the isolated mouse colon (FIG. 3C). Colonic emptying was then modeled as four pellets propagating in the virtual colon (FIG. 3D). Slow transit constipation was simulated by reducing the strength of neuromuscular junction potentials (FIG. 3E). Virtual sacral nerve stimulation was applied as extrinsic, presynaptic events into the biophysical network using burst-patterned stimulation (FIG. 3F) and tonic stimulation (FIG. 3G).

The three-dimensional parameter space of pulse repetition frequency, burst duration, and interburst interval was evaluated to identify the most propulsive pattern of burst stimulation. Burst-patterned stimulation, but not tonic stimulation, increased pellet output and average pellet velocity compared to the slow transit model without stimulation (FIGS. 3H-3I). All ten instances of the model responded successfully to at least one pattern of burst stimulation, where success constituted at least 50% increase in pellet output. Optimizing the pattern of stimulation for each instance of the model (using the most effective pattern of stimulation in a given instance of the model) increased pellet velocity and pellet output in each instance of the model.

### Example 3

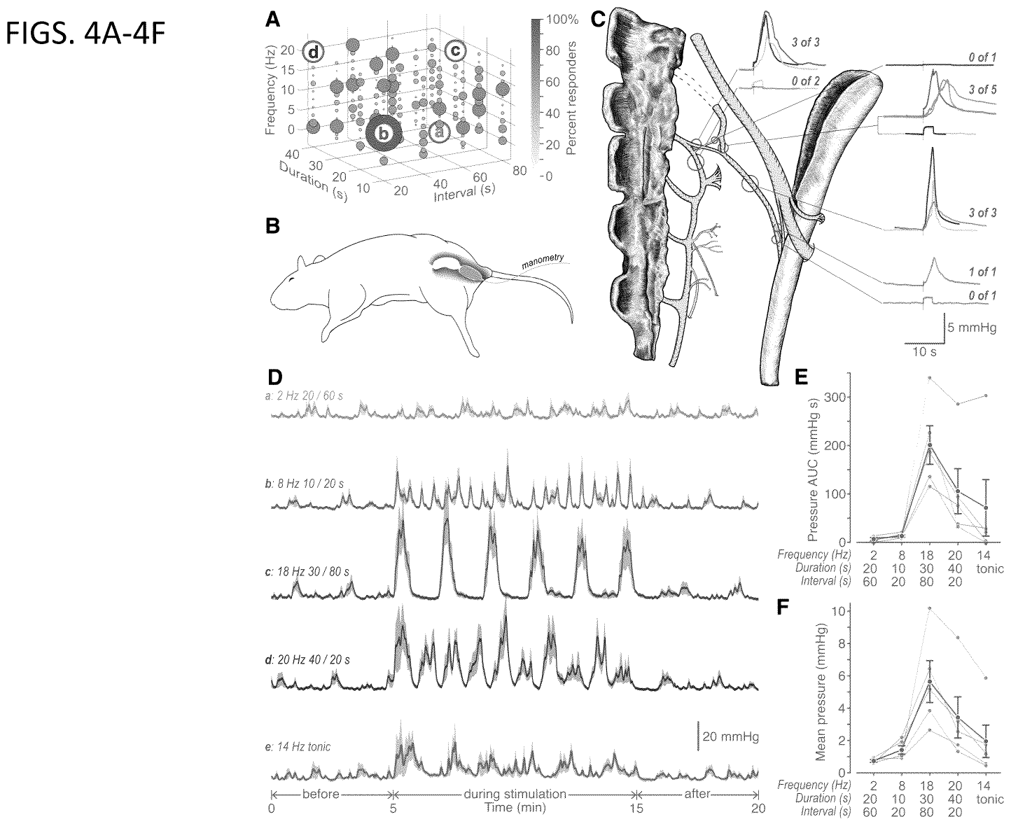

Model-identified burst patterns increase motility in anesthetized rats. For each pattern simulated in the model, the number of successful responders was counted. The best pattern of stimulation produced at least a 50% increase in fecal output across 8 of 10 random instances of the model. The three second best-performing patterns exhibited positive responses in 7 of 10 model instances (FIG. 4A). These four patterns were labeled as model-identified burst patterns and were candidates for further testing and in vivo validation.

Under urethane anesthesia, intraluminal pressure was measured in the rat anorectum at baseline and in response to electrical stimulation of the L6-S1 nerve trunk (the rat homologue of the human S3) (FIGS. 4B-4C). Between the four candidate patterns and tonic stimulation pattern, the burst pattern with 18 Hz, 30 s bursts and 80 s between bursts evoked the most consistent and robust contractions (FIG. 4D). The pattern of sacral nerve stimulation had an effect on the pressure time integral of each contraction (FIG. 4E; n=6, ANOVA F ratio=8.6, p<0.001) and the average pressure of each contraction during stimulation (FIG. 4F; n=6, ANOVA F ratio=8.4, p<0.001). The burst pattern with 18 Hz for 30 s and 80 s interburst interval evoked the largest contractions, and 18 Hz, 30 s on, 80 s off were selected as the most promising stimulation pattern with which to move forward.

### Example 4

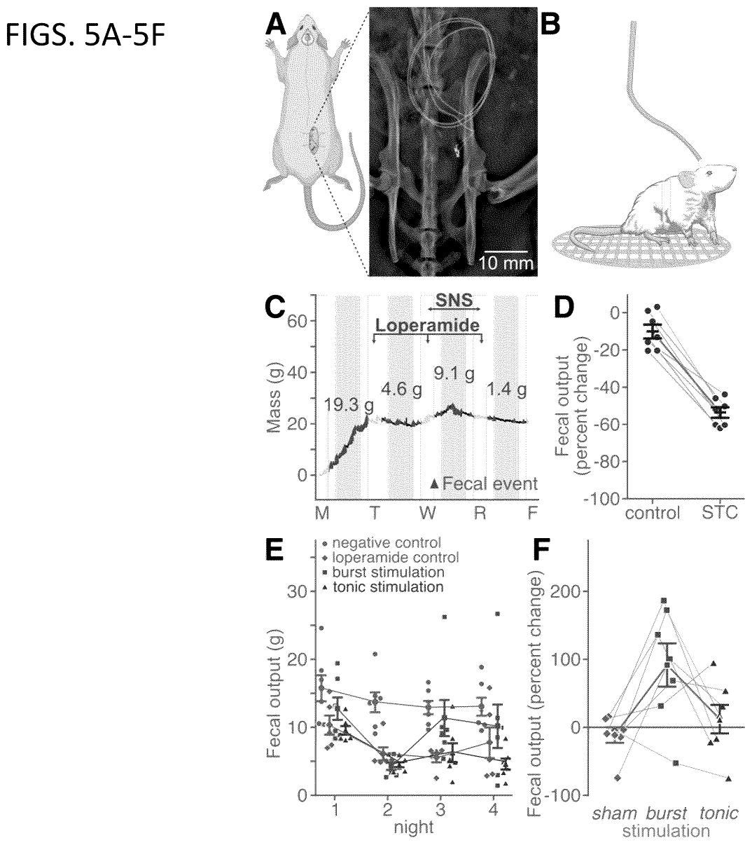

Burst patterns restore fecal output in loperamide model of slow-transit constipation. Changes in fecal output were quantified in awake, behaving rats constipated by loperamide with three conditions: burst stimulation, tonic stimulation, and no stimulation. Rats were surgically implanted with bipolar nerve cuffs on the L6-S1 nerve trunk, and the lead wires were externalized by a threaded, skull-mounted connector (FIG. 5A). Rats were housed in metabolic chambers for five days, and their fecal output was measured overnight from 3:00 pm to the following 9:00 am with 6:00 pm to 6:00 am dark cycle (FIG. 5B). Fecal output over was measured four consecutive nights: baseline on night 1, following midday loperamide gavage on night 2, following loperamide with sacral nerve stimulation (if applicable) on night 3, and following loperamide on night 4 (FIG. 4C). Constipation induced by loperamide gavage reduced fecal output (−54±7.5% with loperamide vs −9.8±9.9% in negative control, FIG. 5D, n=7, t ratio=−10.8, p<0.001). Loperamide also reduced water intake compared to negative control (3.1±43 vs −67±38 mL water per kg rat, n=7, t ratio=3.9, p<0.01) but did not affect body weight (−0.9±1.2 vs 0.7±2.6 percent change in body weight). Loperamide tended to reduce food intake compared to negative control, however the difference was not significant (−28±16 vs 12±46 g chow per kg rat, n=7, t ratio=2.2, p=0.07).

After establishing constipation in the loperamide model on night 2, one of three different interventions was applied on night 3: burst-patterned stimulation, tonic stimulation, or no stimulation. The efficacy of an intervention was quantified as percent change in fecal output from constipated state on night 2 to constipation with treatment on night 3 (FIG. 5E); stimulation pattern had an effect on the change in fecal output (FIG. 5F; n=7, F ratio=5.6, p <0.005). Burst stimulation increased fecal output by 90.7±83.8% compared to 11.6±55.3% with tonic stimulation (p<0.05) and-11.7±29.9% without stimulation (p<0.01).

### Example 5

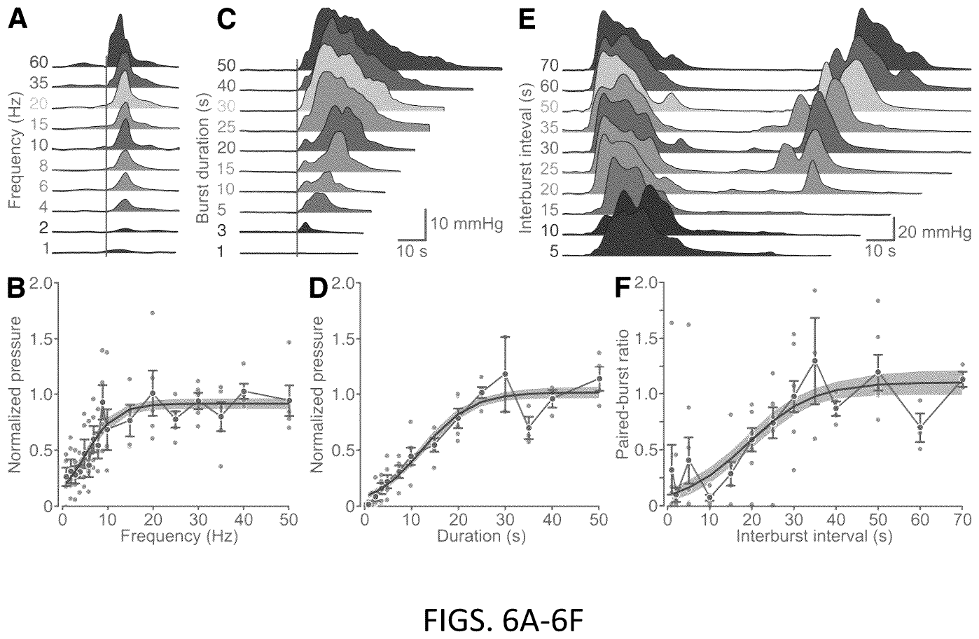

Sensitivity analysis and effects of burst pattern parameters on the anorectum. The robustness of the model-identified burst patterns was assessed by measuring the sensitivity of the anorectal response to changes in stimulation parameters. In the urethane-anesthetized rat, the anorectal response to sacral nerve stimulation increased with increasing pulse repetition frequency for 5 s duration bursts (FIGS. 6A-6B) and increased with increasing burst duration for 20 Hz bursts (FIGS. 6C-6D). Further, there was an interaction between the burst duration and pulse repetition frequency, with longer-duration bursts evoking larger anorectal responses in frequency-matched bursts (n=3, ANOVA F ratio=6.5, p<0.05). These findings are consistent with retrospective comparisons to the computational model of colonic motility. Briefly, effects were found in burst duration (n=10 model instantiations, F ratio=82.7, p<0.0001), burst frequency (n=10, F ratio=9.6, p<0.0001), and interactions between burst duration and burst frequency on the pressure-time integral in the computational model (n=10, F ratio-32.5, p<0.0001).

In the anesthetized rat, the effect of the interburst interval was characterized in a paired-burst ratio experiment. Here, two identical bursts of sacral nerve stimulation were delivered, 20 Hz for 40 s, while varying the delay between the pair of bursts. The rationale was that a sufficiently long delay would evoke two near-identical responses in the anorectum, whereas a shorter delay period between bursts would evoke larger or smaller responses due to facilitation or depression, respectively (FIG. 6E). Sufficiently long interburst intervals yielded consistent responses between the first and second burst of stimulation and decreasing the interburst interval decreased the pressure time integral of the anorectal response relative to the first burst of stimulation (FIG. 5F). These data were fitted with three-parameter logistic functions, and the minimum parameters necessary to evoke the maximum response were determined as the inverse estimate for 95% of the asymptote, bounded by the 95% confidence interval. The minimum value for each parameter to achieve the maximal anorectal response was 15 [10, 20] Hz pulse repetition frequency, 25 [19, 32] s burst duration, and 31 [23, 39] s interburst interval.

### Example 6

Pelvic nerve stimulation in the isolated mouse colon. Pelvic nerve stimulation evoked calcium waves associated with colonic motor complexes (CMCs) in the isolated mouse colon. Calcium waves evoked by pelvic nerve stimulation were similar to those evoked by fluid distension, and the evoked waves had similar latency, propagation distance, and propagation speed across three conditions: fluid distension, and pelvic nerve stimulation in the voided and distended colons (FIG. 7; Table 17). The frequency during stimulation was measured and compared to a 5 min baseline period. The frequency of calcium waves was greater when the colon was constantly distended compared to the voided colon (1.01±0.53 vs. 0.36±0.27 min⁻¹; n=10, t Ratio=−3.63, p-value=0.006), and the analysis was separated by colon state: distended and voided. Tonic pelvic nerve stimulation evoked fewer calcium waves than burst pelvic nerve stimulation in the voided colon (n≥10, F Ratio=3.3, p-value=0.03), but not the distended colon (n≥8, F Ratio=1.68, p-value=0.2). Pelvic nerve stimulation at 20 Hz and 400 μs pulse width, with 60 s and 40 s inter-burst intervals, increased the frequency of calcium waves more than tonic stimulation at 14 Hz and 210 μs pulse width (FIGS. 7D-7E; Table 18).

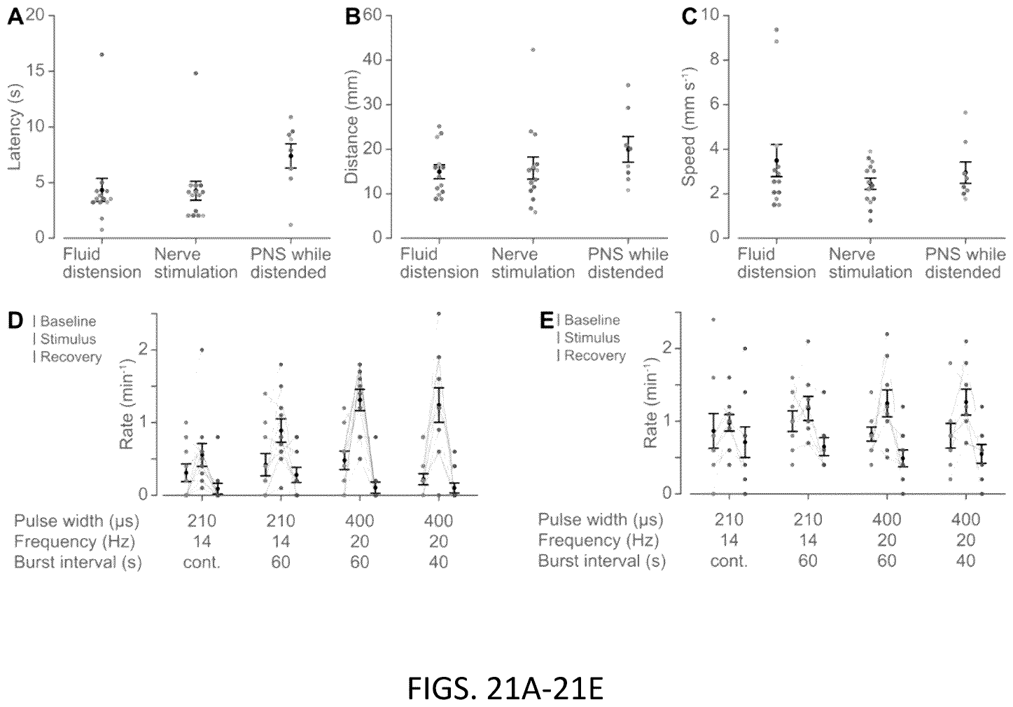

Pelvic nerve stimulation evoked myoelectric colonic motor complexes (CMCs) in the isolated mouse colon (FIGS. 21A-21B). The effect of burst duration and frequency on stimulation threshold were evaluated individually while holding constant the frequency, burst duration, and pulses per burst (FIGS. 21C-21E). Stimulation threshold decreased with increasing pulses per burst up to 150 pulses per burst (n≥6, X²=18.21, p-value=0.001) and with increasing frequency (n≥6, X²=8.57, p-value=0.01). However, an effect of burst duration at constant pulses per burst on stimulation threshold was not observed (n≥6, X²=0.98, p-value=0.6). Stimulation patterns with 5 to 7.5 s bursts had the lowest stimulation threshold among patterns using 20 Hz stimulation (Table 19), and 20 Hz had the lowest threshold among patterns containing 5 s bursts (Table 20).

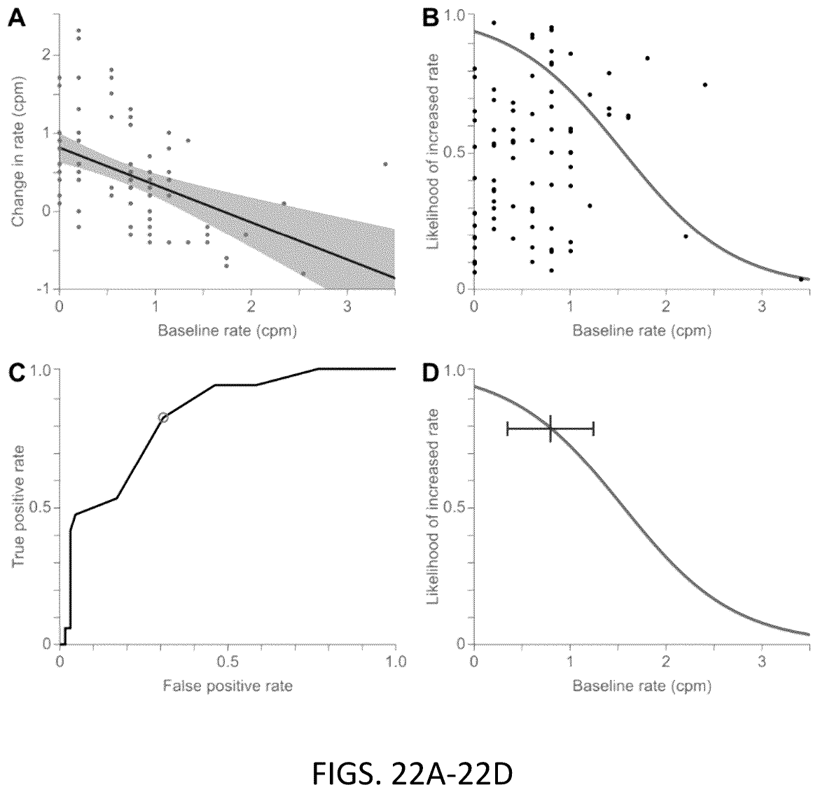

The effect of stimulation, i.e., the likelihood of increasing the rate of calcium waves, was dependent on the ongoing rate of calcium waves (n=82, F Ratio=19.0, p-value<0.0001). The change in rate (cycles per minute, cpm) during pelvic nerve stimulation from baseline was negatively correlated with the baseline rate of calcium waves (FIG. 22). A logistic fit was applied to the Boolean expression “change in rate is non-negative” against the baseline rate across all technical replicates. The logistic fit was statistically significant (n=82, X²=13.01, p-value=0.0003), and the median likelihood of a non-negative change in rate occurred when the baseline rate was 1.54 [1.13, 3.02] cpm. Colon preparations with baseline rate less than 0.8 [0.33, 1.25] cpm were most likely to respond with a positive change in rate to pelvic nerve stimulation, and colons with baseline rate greater than 0.8 cpm were most likely to respond with a negative change in rate to pelvic nerve stimulation.

TABLE 1
Ionic currents of model cells.

| Cell type | Number | Ionic Currents |
|---|---|---|
| S-type neuron | 731 | K_{A}, K_{dr}, K_{M}, Na_{v1.3}, Na_{v1.7} |
| AH-type neuron | 250 | Ca_{N}, Iₕ, K_{A}, K_{CaF}, K_{CaS}, K_{dr}, Na_{v1.3}, Na_{v1.7}, Na_{v1.9}, |
| | | NSCC_{Ca} |
| Enterochromaffin cell | 100 | Artificial spike generator (0.4-1.25 Hz) |
| Enteric glia cell | 99 | K_{v1.1}, K_{v1.2}, K_{ir2.3}, Na_{v1.5} |
| Smooth muscle fiber | 60 | Ca_{L}, Ca_{T}, K_{fi}, Kₙᵢ, K_{sd}, Na—K, Na_{v1.5}, NCX |
| Interstitial cell of Cajal | 50 | Ano₁, Ca_{T}, K_{b}, Na_{b}, Naᵥ, Na_{v1.5}, NSV, SOC, |
| | | SERCA, PMCA |

TABLE 2
Neuron population subsets identified in a single-
cell atlas of the mouse enteric nervous system.

| | | | | | | Projection | |
|---|---|---|---|---|---|---|---|
| Model | Atlas | Abundance | | | | distance (mm) | |
| Subset | Subset | (%) | Neurotransmitters | Receptors | Type | Mean | SD |
| IPAN 1 | PSN 1 | 16.7 | aCh, CGRP | Calcrl, Adra2a, SP, | AH | 0.7 | 0.35 |
| | | | | VIP | | | |
| IPAN 2 | pSN 2 | 8.6 | aCh, CGRP, Cck | SP | AH | 0.7 | 0.35 |
| DIN 1^{†} | PSN 3^{†} | 10.8 | aCh, CGRP, Cck, | Calcrl, Adra2a | S | 5.2 | 0.33 |
| | | | VIP | | | | |
| DIN 3 | pSN 4 | 9.6 | aCh, CGRP, Sst | CckBR, Adra2a, | S | 5.2 | 0.33 |
| | | | | Sstr1, VIP | | | |
| AI 1^{†} | PIN 1^{†} | 3.8 | aCh, NE, Enk | CckAR, Calcrl, | S | −5.9 | 0.33 |
| | | | | Adra2a, VIP | | | |
| AI 2^{†} | pIN 2^{†} | 3 | aCh, Enk, SP | Calcrl, Adra1a, | S | −5.9 | 0.33 |
| | | | | Adra2a | | | |
| AI 3^{†} | pIN 3^{†} | 1.7 | aCh, Enk, SP | Calcrl, Adra2a, | S | −5.9 | 0.33 |
| | | | | Sstr1, VIP | | | |
| IMN 1 | pIMN 1 | 2.4 | NO, VIP | Sstr2, VIP | S | 3.6 | 0.35 |
| IMN 2 | pIMN 2 | 4.2 | NO, VIP | Calcrl, VIP | S | 3.6 | 0.35 |
| IMN 3 | pIMN 3 | 1.9 | NO, NE, VIP | Calcrl, VIP | S | 3.6 | 0.35 |
| IMN 4 | pIMN 4 | 6.6 | NO | VIP | S | 3.6 | 0.35 |
| IMN 5 | pIMN 5 | 4.5 | NO | VIP | S | 3.6 | 0.35 |
| IMN 6 | pIMN 6 | 6.4 | NO | Calcrl, VIP | S | 3.6 | 0.35 |
| IMN 7 | pIMN 7 | 0.5 | NO | VIP | S | 3.6 | 0.35 |
| EMN 1 | pEMN 1 | 3.5 | aCh, Enk, SP | Calcrl, VIP | S | −2.3 | 0.37 |
| EMN 2 | pEMN 2 | 2.7 | aCh, Enk, SP | Calcrl, Sstr1, SP, | S | −2.3 | 0.37 |
| | | | | VIP | | | |
| EMN 3 | pEMN 3 | 6.9 | aCh, Enk, SP | Sstr1, VIP | S | −2.3 | 0.37 |
| EMN 4 | pEMN 4 | 3.5 | aCh, Enk, SP | Calcrl, Sstr1, SP, | S | −2.3 | 0.37 |
| | | | | VIP | | | |
| EMN 5 | pEMN 5 | 3.1 | aCh, Enk, SP | Calcrl, Sstr1, SP, | S | −2.3 | 0.37 |
| | | | | VIP | | | |
| | pSVN 1 | | NE, VIP | Calcrl, Sstr1, Sstr2, | | | |
| | | | | SP, VIP | | | |
| | pSVN 2 | | NO | Calcrl, Sstr1, VIP | | | |

Mechanosensitive subsets are indicated by a dagger (^{†}). aCh, acetylcholine; Enk, enkephalin; SP, substance P; NO, nitric oxide; VIP, vasoactive intestinal peptide; NE, norepinephrine; Cck, cholecystokinin; Sst, somatostatin; Calcrl, calcitonin receptor-like receptor; Sstr1, somatostatin receptor 1; Sstr2, somatostatin receptor 2; CckAR, cholecystokinin receptor type A; Adra2a, alpha-2A adrenergic receptor; Adra1a, alpha-1A adrenergic receptor; CckBR, cholecystokinin receptor type B. The neuron type (AH- and S- type neurons) and projection distance (mm) are given for each model subset.

TABLE 3
Pressure-independent parameters of the Nav1.5 model.

| Parameter | Minimum | Maximum | Calibrated Value |
|---|---|---|---|
| n (number μm⁻²) | 1 | 500 | 14.6 |
| C→C_{A} b (ms⁻¹) | 0.001 | 500 | 258. |
| C→C_{A} h (mV) | −120 | 20 | 2.99 |
| C→C_{A} k (mV) | −50 | 50 | −6.10 |
| C_{A}→C b (ms⁻¹) | 0.001 | 500 | 229. |
| C_{A}→C h (mV) | −120 | 20 | −37.4 |
| C_{A}→C k (mV) | −50 | 50 | 12.5 |
| C_{A}→I_{A} b (ms⁻¹) | 0.001 | 500 | 0.001 |
| C_{A}→I_{A} h (mV) | −120 | 20 | −49.4 |
| C_{A}→I_{A} k (mV) | −50 | 50 | 21.4 |
| I_{A}→C_{A} b (ms⁻¹) | 0.001 | 500 | 238. |
| I_{A}→C_{A} h (mV) | −120 | 20 | −120 |
| I_{A}→C_{A} k (mV) | −50 | 50 | 14.3 |
| I_{O}→O b (ms⁻¹) | 0.001 | 500 | 69.7 |
| I_{O}→O h (mV) | −120 | 20 | −120 |
| I_{O}→O k (mV) | −50 | 50 | 7.62 |
| O→I_{O} b (ms⁻¹1) | 0.001 | 500 | 293. |
| O→I_{O} h (mV) | −120 | 20 | −80.6 |
| O→I_{O} k (mV) | −50 | 50 | 13.7 |
| C_{A}→O R (ms⁻¹) | 0.01 | 100 | 62.6 |
| O→C_{A} R (ms⁻¹) | 0.01 | 100 | 53.4 |
| I_{A}→I_{O} R (ms⁻¹) | 0.01 | 100 | 100 |
| I_{O}→I R (ms⁻¹) | 0.01 | 100 | 56.2 |

TABLE 4
Pressure-sensitive parameters of the Nav1.5 model.

| Parameter | Minimum | Maximum | Calibrated Value |
|---|---|---|---|
| C→C_{A} Pₕ | −1 | 1 | −0.328 |
| (mV/mmHg) | | | |
| C→C_{A} Pₖ | −0.5 | 0.5 | 0.00223 |
| (mV/mmHg) | | | |
| C_{A}→C Pₕ | −1 | 1 | −0.330 |
| (mV/mmHg) | | | |
| C_{A}→C Pₖ | −0.5 | 0.5 | −0.148 |
| (mV/mmHg) | | | |

TABLE 5
Two-state Boltzmann model target parameters.

| | |
|---|---|
| Activation | Inactivation |

| Pressure | mx | hB | kB | mx | hB | kB |
|---|---|---|---|---|---|---|
| (mmHg) | (pA) | (mV) | (mV) | (pA) | (mV) | (mV) |
| 0 | 28.3 | −33.0 | −5.6 | 30.4 | −55.6 | 11.0 |
| −10 | 30.4 | −40.6 | −5.8 | 32.2 | −63.2 | 11.1 |
| −20 | 33.5 | −47.6 | −6.2 | 32.9 | −70.2 | 11.6 |
| −40 | 36.0 | −61.7 | −6.4 | 29.0 | −84.3 | 11.8 |
| −50 | 36.3 | −68.5 | −6.8 | 23.1 | −91.1 | 12.1 |

TABLE 6
Calibration parameters of the ICC model.

| | Minimum | Maximum | Calibrated Value |
|---|---|---|---|
| Parameter | (10^{x}) | (10^{x}) | (10^{x}) |
| Na_{v1.5} n | −7 | 0 | −0.911 |
| (number μm⁻²) | | | |
| Ano₁ g̅ (nS) | −7 | 3 | −1.93 |
| Ca_{T} g̅ (nS) | −7 | 3 | −1.98 |
| K_{b} g̅ (nS) | −7 | 3 | −0.861 |
| Na_{b} g̅ (nS) | −7 | 3 | −1.25 |
| SOC g̅ (nS) | −7 | 3 | −0.873 |
| Ano₁ O₀ | −7 | 0 | −3.40 |
| IPR P₀ | −7 | 0 | −0.315 |
| [Ca²⁺]_{i, 0} (mM) | −7 | 0 | −3.70 |
| [Ca²⁺]_{ER, 0} (mM) | −7 | 0 | −0.699 |

TABLE 7
Target parameters for mechanosensitive enteric neurons based on E.
Drokhlyansky et al., The Human and Mouse Enteric Nervous System at
Single-Cell Resolution. Cell 182, 1606-1622.e1623 (2020).

| | | Target (θₓ) | | |
|---|---|---|---|---|
| | Symbol | AH | S | S |
| Parameter | (x) | RAMEN | RAMEN | SAMEN |
| Firing rate at baseline | b | 0 | 0 | 0 |
| Firing rate during dynamic stretch phase | d | 9.4 | 9.4 | 8.3 |
| Firing rate during static stretch phase | s | 5.4 | 5.4 | 4.3 |
| Time of last action potential | t | 1 | 1 | 10 |
| Resting membrane potential | V | −47.5 | −47.5 | −47.5 |
| Maximum inter-spike interval | Δ | 0 | 0 | 0 |

TABLE 8
Parameters for computational fluid dynamics.

| Parameter | Symbol | Value | |
|---|---|---|---|
| Eulerian grid size | Nx, N_{Y} | 256, 128 | |
| Lagrangian grid size | L_{X}, L_{Y} | 150 mm, 30 mm | |
| Dynamic viscosity | μ | 0.001 | Pa s |
| Density | ρ | 1 | kg m⁻³ |
| Time step | dt | 5 | ms |
| Hookean spring stiffness | k_{SCproximal}, k_{SCdistal} | 3.7, 3.0 | kPa |
| colon | | | |
| Torsion constant colon | k_{BCproximal}, k_{BCdistal} | 7.1, 31.3 | kPa |
| Hookean spring stiffness | k_{SP} | 100 | kPa |
| pellet | | | |
| Torsion constant pellet | k_{BP} | 1 | MPa |
| Maximum contractile force | Fₘₐₓ | 416 | mN mm⁻² |

TABLE 9
Myogenic PSO parameters.

| Parameter | Symbol | Value |
|---|---|---|
| # particles | — | 130 |
| # seeds | s | 3 |
| # neighborhoods | — | 10 |
| # swarms | — | 5 |
| maximum velocity | νₘₐₓ | 0.1 |
| inertia weight | w | 1.2 |
| decay | d | 20 iterations |

TABLE 10
Calibrated myogenic parameters.

| | | Lower | Upper | | Calibrated |
|---|---|---|---|---|---|
| Parameter | Units | bound | bound | Scale | value |
| ICC_{MY} ↔ | nS | 10⁻⁴ | 10¹ | Log | 0.00562 |
| ICC_{MY} | | | | | |
| ICC_{MY} ↔ | nS | 10⁻⁴ | 10¹ | Log | 0.0516 |
| CMF | | | | | |
| ICC_{MY} ↔ | nS | 10⁻⁴ | 10¹ | Log | 0.000108 |
| LMF | | | | | |
| ICC_{IMC} ↔ | nS | 10⁻⁴ | 10¹ | Log | 1.13 |
| CMF | | | | | |
| ICC_{IML} ↔ | nS | 10⁻⁴ | 10¹ | Log | 1.79 |
| LMF | | | | | |
| ICC_{SMP} ↔ | nS | 10⁻⁴ | 10¹ | Log | 0.00779 |
| ICC_{SMP} | | | | | |
| ICC_{SMP} ↔ | nS | 10⁻⁴ | 10¹ | Log | 0.105 |
| CMF | | | | | |
| CMF↔ CMF | nS | 10⁻⁴ | 10¹ | Log | 0.451 |
| LMF ↔ LMF | nS | 10⁻⁴ | 10¹ | Log | 0.163 |
| g̅_{sdk} | S cm⁻² | 5.3 | 530 | Log | 102. |
| g̅ₙₐᵥ₁₅ | pS | 1 | 100 | Log | 19.3 |
| ICC_{MY} | min⁻¹ | 0.5 | 4 | Linear | 2.53 |
| frequency | | | | | |
| ICC_{IM} | min⁻¹ | 18 | 20 | Linear | 19.2 |
| frequency | | | | | |
| ICC_{SMP} | min⁻¹ | 14 | 18 | Linear | 14.8 |
| frequency | | | | | |
| # ICC_{MY} | cells | 20 | 200 | Linear | 89 |
| # ICC_{IMC} | cells | 20 | 200 | Linear | 89 |
| # ICC_{IML} | cells | 20 | 200 | Linear | 89 |
| # ICC_{SMP} | cells | 20 | 200 | Linear | 89 |

TABLE 11
Target characteristics of myogenic activity.

| Pa- | | | | | | | | Per- |
|---|---|---|---|---|---|---|---|---|
| ram- | | | | Lower | Upper | | | cent |
| eter | Units | μ | σ | bound | bound | μ | σ | error |
| v_{cm} | mV | −44.9 | 5.5 | −72.4 | −17.4 | −40.3 | 0.53 | 10% |
| r_{cm} | min⁻¹ | 4.6 | 1.1 | −0.9 | 10.1 | 25.9 | 7.74 | 463% |
| k_{cm} | spikes | 5.5 | 1.1 | 0 | 11 | 3.8 | 0.49 | −31% |
| f_{cm} | min⁻¹ | 23.8 | 4.0 | 3.8 | 43.8 | 42.1 | 3.2 | 77% |
| vₗₘ | mV | −48.1 | 7.2 | −84.1 | −12.1 | −70.2 | 1.46 × | −167% |
| | | | | | | | 10⁻⁷ | |
| rₗₘ | min⁻¹ | 4.3 | 1.3 | −2.2 | 10.8 | 0 | — | — |
| kₗₘ | spikes | 6 | 0.9 | 1.5 | 10.5 | 0 | — | — |
| fₗₘ | min⁻¹ | 23.9 | 5.3 | 2.6 | 50.4 | 0 | — | — |
| d | s | 2.6 | 0.3 | 1.1 | 4.1 | 1.4 | 0.1 | −46% |

TABLE 12
Presynaptic parameters included in fluid distension parameter
optimization. In some cases, the receptor is listed; in fact,
the tuned parameter was the synaptic weight of the neurotransmitter
acting on the postsynaptic population (columns).

| | Postsynaptic population | | | | | | | |
|---|---|---|---|---|---|---|---|---|
| Presynaptic | | | | | | IMN | | |
| Population | | | | | | IMN | IMN | IMN |
| ↓ | ACI | DNI | DEI | EMN | Glia | 5 | 6 | 7 |
| ACI | p | p | p | p | SIJP | p, | p, | p, |
| | | | | | | Enk | Enk | Enk |

| | DNI | Glia | IMN |
|---|---|---|---|
| DNI | p, nAChR | mAChR | p, nAChR |

| | Glia |
|---|---|
| DEI | mAChR |

| | IPAN |
|---|---|
| ECC | f5HT-1p, s5HT-1p |

| | ICC-IMC | ICC-IML | ICC-MY | CMF | LMF |
|---|---|---|---|---|---|
| EMN | p, Enk | p, Enk | p, Enk | p, Enk | p, Enk |

| | ACI | DNI | Glia |
|---|---|---|---|
| IPAN | p, nAChR | p, nAChR | mAChR |

Abbreviations: ACI, ascending cholinergic interneuron; DNI, descending nitrergic interneuron; DEI, descending excitatory interneuron; EMN, excitatory motor neuron; IMN, inhibitory motor neuron; ECC, enterochromaffin cell; IPAN, intrinsic primary afferent neuron; ICC, interstitial cells of Cajal; IMC, intramuscular circular; IML, intramuscular longitudinal; MY, myenteric; CMF, circular muscle fiber; LMF, longitudinal muscle fiber; p, probability; sIJP, slow inhibitory junction potential; fIJP, fast inhibitory junction potential; Enk, enkephalin; nAChR, nicotinic cholinergic receptor; mAChR, muscarinic cholinergic receptor; f5HT-1p, fast serotonergic receptor; s5HT-1p, slow serotonergic receptor.

TABLE 13
Postsynaptic parameters included in fluid
distension parameter optimization.

| Postsynaptic | |
|---|---|
| population | Neuropeptide |
| ICC-IMC | SP |
| ICC-IML | SP |
| ICC-MY | SP |
| ICC-SMP | SP |
| EMN 1 | SP |
| EMN 2 | SP |
| EMN 3 | SP |
| EMN 4 | SP |
| EMN 5 | SP |
| ACI 1 | CGRP, SP, VIP |
| ACI 2 | CGRP, SP, VIP |
| ACI 3 | CGRP, SP, VIP |
| DNI | CGRP, SP |
| DEI | CGRP, SP |
| CMF | SP |
| LMF | SP |

Abbreviations: ACI, ascending cholinergic interneuron; DNI, descending nitrergic interneuron; DEI, descending excitatory interneuron; EMN, excitatory motor neuron; ICC, interstitial cells of Cajal; IMC, intramuscular circular; IML, intramuscular longitudinal; MY, myenteric; SMP, submucosal plexus; CMF, circular muscle fiber; LMF, longitudinal muscle fiber; SP, substance P; CGRP, calcitonin gene-related peptide; VIP, vasoactive intestinal peptide.

TABLE 14
Mechanical parameters included in pellet velocity parameter optimization.
ECC brush response

Maximum contractile force
Torsional spring constant- proximal colon
Torsional spring constant- distal colon
Torsional spring constant- pellet
Hookean spring constant- proximal colon
Hookean spring constant- distal colon
Hookean spring constant- pellet
Gap between colonic wall & pellet boundary

TABLE 15
Pellet velocity and percent error for increasing pellet
diameter. Velocity is reported as mean ± SEM.

| Pellet diameter | Velocity (mm s⁻¹) | | | Percent error (%) | |
|---|---|---|---|---|---|
| (mm) | Mouse (57) | Optimization | Validation | Optimization | Validation |
| 1.0 | 0.29 ± 0.10 | 0.27 ± 0.08 | 0.42 ± 0.02 | −7.4 ± 29.2 | 45.0 ± 6.3 |
| 1.5 | 0.40 ± 0.08 | 0.40 ± 0.02 | 0.35 ± 0.02 | −1.3 ± 3.8 | −13.2 ± 6.1 |
| 2.0 | 0.55 ± 0.07 | 0.40 ± 0.15 | 0.42 ± 0.02 | −26.9 ± 27.1 | −23.8 ± 3.4 |
| 2.5 | 0.72± | | 0.26 ± 0.01 | | −64.0 ± 1.4 |
| 3.0 | 0.91± | | 0.05 ± 0.01 | | −94.6 ± 1.1 |

TABLE 16
Pelvic innervation calibration.

| Parameter | Units | Minimum | Maximum | Scale | Value |
|---|---|---|---|---|---|
| Center | mm | 24 | 40 | Linear | 31.4 |
| Half-span | mm | 4 | 24 | Linear | 20.9 |
| p ACI | % | 0 | 1 | Linear | 70.1 |
| w ACI | μS | 0.01 | 100 | Log | 0.84 |
| p DNI | % | 0 | 1 | Linear | 25.6 |
| w DNI | μS | 0.01 | 100 | Log | 0.23 |
| p DEI | % | 0 | 1 | Linear | 50.8 |
| w DEI | μS | 0.01 | 100 | Log | 2.97 |
| p EMN | % | 0 | 1 | Linear | 2.3 |
| w EMN | μS | 0.01 | 100 | Log | 1.05 |
| p IMN | % | 0 | 1 | Linear | 45.8 |
| w IMN | μS | 0.01 | 100 | Log | 41.8 |
| p IPAN | % | 0 | 1 | Linear | 91.0 |
| w IPAN | μS | 0.01 | 100 | Log | 1.3 |

Abbreviations: ACI, ascending cholinergic interneuron; DNI, descending nitrergic interneuron; DEI, descending excitatory interneuron; EMN, excitatory motor neuron; IMN, inhibitory motor neuron; IPAN, intrinsic primary afferent neuron; p, probability; w, weight.

TABLE 17
Similarities between colonic motor complexes evoked by distension and electrical stimulation.

| | Lognormal | | ANOVA | | | | | | 1^{st}, 3^{rd} |
|---|---|---|---|---|---|---|---|---|---|
| | A2 | p-value | F | p-value | | Scale μ | Shape σ | Median | quartile |
| Latency (s) | 0.48 | 0.23 | 2.65 | 0.09 | Fluid distension | 1.2 | 0.65 | 3.5 | 3.2, 4.3 |
| | | | | | Pelvic nerve stimulation | 1.3 | 0.52 | 4.0 | 2.1, 4.7 |
| | | | | | (voided) | | | | |
| | | | | | Pelvic nerve stimulation | 1.9 | 0.67 | 8.4 | 5.6, 9.4 |
| | | | | | (distended) | | | | |
| Duration (mm) | 0.21 | 0.88 | 1.35 | 0.27 | Fluid distension | 2.6 | 0.37 | 13.2 | 9.4, 19.1 |
| | | | | | Pelvic nerve stimulation | 2.6 | 0.53 | 13.4 | 9.5, 17.5 |
| | | | | | (voided) | | | | |
| | | | | | Pelvic nerve stimulation | 2.9 | 0.38 | 17.5 | 13.0, 26.5 |
| | | | | | (distended) | | | | |
| Velocity (mm s⁻¹) | 0.43 | 0.3 | 0.93 | 0.41 | Fluid distension | 1.1 | 0.57 | 2.6 | 1.9, 3.7 |
| | | | | | Pelvic nerve stimulation | 0.8 | 0.44 | 2.4 | 1.7, 3.3 |
| | | | | | (voided) | | | | |
| | | | | | Pelvic nerve stimulation | 1.0 | 0.38 | 2.5 | 2.0, 4.0 |
| | | | | | (distended) | | | | |

TABLE 18
Increase in the rate of calcium waves from baseline.

| | Stimulation pattern | | t | | | | Effect |
|---|---|---|---|---|---|---|---|
| | Group 1 | Group 2 | Ratio | p-value | α_{adj} | H₀ | Size |
| Voided | Cont. 14 Hz, 210 μs | 60 s, 14 Hz, 210 μ;s | −0.55 | 0.6 | 0.0042 | Accept | −0.2 |
| | Cont. 14 Hz, 210 μs | 60 s, 20 Hz, 400 μs | −3.25 | 0.01 | 0.05 | Reject | −1.15 |
| | Cont. 14 Hz, 210 μs | 40 s, 20 Hz, 400 μs | −3.03 | 0.02 | 0.025 | Reject | −1.07 |
| | 60 s, 14 Hz, 210 μs | 60 s, 20 Hz, 400 μs | −1.33 | 0.22 | 0.0071 | Accept | −0.47 |
| | 60 s, 14 Hz, 210 μs | 40 s, 20 Hz, 400 μs | −2.32 | 0.05 | 0.0167 | Accept | −0.82 |
| | 60 s, 20 Hz, 400 μs | 40 s, 20 Hz, 400 μs | 0.61 | 0.56 | 0.005 | Accept | 0.22 |
| Distended | Cont. 14 Hz, 210 μs | 60 s, 14 Hz, 210 μs | −0.58 | 0.58 | 0.0045 | Accept | −0.2 |
| | Cont. 14 Hz, 210 μs | 60 s, 20 Hz, 400 μs | −1.34 | 0.22 | 0.0083 | Accept | −0.47 |
| | Cont. 14 Hz, 210 μs | 40 s, 20 Hz, 400 μs | −1.70 | 0.13 | 0.0125 | Accept | −0.6 |
| | 60 s, 14 Hz, 210 μs | 60 s, 20 Hz, 400 μs | −0.76 | 0.47 | 0.0056 | Accept | −0.27 |
| | 60 s, 14 Hz, 210 μs | 40 s, 20 Hz, 400 μs | −1.05 | 0.33 | 0.0063 | Accept | −0.37 |
| | 60 s, 20 Hz, 400 μs | 40 s, 20 Hz, 400 μs | 1.34 | 0.22 | 0.01 | Accept | 0.47 |

TABLE 19
The effect of burst duration on stimulation threshold at 20 Hz.

| Burst duration (s) | | | | | | Effect |
|---|---|---|---|---|---|---|
| Group 1 | Group 2 | D | p-value | α_{adj} | H₀ | Size |
| 2.5 | 3.75 | 0.61 | 0.09 | 0.01 | Accept | 0.23 |
| 2.5 | 5 | 0.79 | 0.003 | 0.025 | Reject | 0.39 |
| 2.5 | 7.5 | 0.9 | 0.0002 | 0.05 | Reject | 0.51 |
| 2.5 | 10 | 0.39 | 0.34 | 0.0056 | Accept | 0.1 |
| 3.75 | 5 | 0.57 | 0.13 | 0.0083 | Accept | 0.18 |
| 3.75 | 7.5 | 0.73 | 0.02 | 0.0125 | Accept | 0.32 |
| 3.75 | 10 | 0.23 | 0.96 | 0.005 | Accept | 0.19 |
| 5 | 7.5 | 0.5 | 0.17 | 0.0071 | Accept | 0.18 |
| 5 | 10 | 0.5 | 0.17 | 0.0071 | Accept | 0.17 |
| 7.5 | 10 | 0.7 | 0.01 | 0.0167 | Reject | 0.36 |

TABLE 20
The effect of burst frequency on stimulation
threshold with 5 s bursts.

| Frequency (Hz) | | | | | | Effect |
|---|---|---|---|---|---|---|
| Group 1 | Group 2 | D | p-value | α_{adj} | H₀ | Size |
| 5 | 5 | 0.75 | 0.23 | 0.0167 | Accept | 0.15 |
| 5 | 20 | 1 | 0.007 | 0.05 | Reject | 0.53 |
| 10 | 20 | 0.6 | 0.19 | 0.025 | Accept | 0.18 |

TABLE 21
Peak and time-to-peak pressure response in the model as
function of the number of pulses delivered at 20 Hz.

| Number of pulses | Maximum pressure (Pa) | Time-to-peak (s) |
|---|---|---|
| 50 | 57.6 ± 6.2 | 5.4 ± 1.5 |
| 75 | 53.2 ± 2.4 | 2.6 ± 0.3 |
| 100 | 53 ± 4.5 | 3.2 ± 0.6 |
| 150 | 58 ± 2.9 | 4.9 ± 2 |
| 200 | 53.7 ± 1.8 | 2.6 ± 0.8 |

TABLE 22
Average rectified electromyogram (EMG) of CMCs evoked
by 20 Hz stimulation in the isolated mouse colon.

| Number of pulses | Mean rectified EMG (μV) |
|---|---|
| 50 | 18.4 ± 1.9 |
| 75 | 15.4 ± 1.4 |
| 100 | 24.1 ± 1.8 |
| 150 | 21.7 ± 2.2 |
| 200 | 18.5 ± 1.1 |

TABLE 23
Excitation-contraction coupling parameters.

| Parameter | Units | Value |
|---|---|---|
| α | mN mm⁻² | 416 |
| n | | 3.5 |
| K_{d} | μM | 1 |
| t1 | s | 4 |
| t2 | s | 0.235 |

1. A method of treating a gastrointestinal dysmotility disorder in a subject in need thereof, the method comprising:
applying a temporal pattern of electrical stimulation comprising burst-patterned stimulation to a target nerve or a set of target nerves in a subject having at least one symptom of a gastrointestinal dysmotility disorder, wherein application of the temporal pattern of electrical stimulation modulates gastrointestinal motility in the subject.

2-5. (canceled)

6. The method of claim 3, wherein the pulse repetition frequency is from about 0.1 Hz to about 30 Hz.

7. (canceled)

8. The method of claim 3, wherein the burst duration is from about 10 seconds to about 60 seconds.

9. (canceled)

10. The method of claim 3, wherein the interburst interval is from about 10 seconds to about 120 seconds.

11-15. (canceled)

16. The method of claim 1, wherein the gastrointestinal motility disorder comprises colonic dysmotility and/or constipation.

17-21. (canceled)

22. The method of claim 1, wherein the target nerve or a set of target nerves comprises the sacral nerve, and wherein the gastrointestinal dysmotility disorder comprises constipation.

23. The method of claim 1, wherein the target nerve or a set of target nerves comprises the sacral nerve, and wherein the gastrointestinal dysmotility disorder comprises disorders of gut-brain interactions.

24. The method of claim 1, wherein the at least one symptom of a gastrointestinal dysmotility disorder comprises early satiety, nausea, vomiting, bloating, diarrhea, constipation, involuntary weight loss, abdominal pain, abdominal swelling (distention), and/or intrarectal pressure.

25-27. (canceled)

28. The method of claim 1, further comprising:
programming a pulse generator to output the temporal pattern of electrical stimulation, wherein the step of applying the temporal pattern of electrical stimulation is performed by delivering the temporal pattern of electrical stimulation to the subject from the pulse generator.

29-37. (canceled)

38. A method of selecting a temporal pattern of electrical stimulation to treat a gastrointestinal dysmotility disorder in a human subject in need thereof, the method comprising:
delivering a first burst-patterned stimulation to a target nerve or a set of target nerves in a subject having at least one symptom of a gastrointestinal dysmotility disorder and assessing efficacy of stimulation and/or a degree of relief of the at least one symptom;
determining a second burst-patterned stimulation by adjusting a stimulation parameter of the first burst-patterned stimulation;
delivering the second burst-patterned stimulation to the target nerve or the set of target nerves in the subject and reassessing the efficacy of stimulation and/or the degree of relief of the at least one symptom; and
selecting for treatment one of the first burst-patterned stimulation or the second burst-patterned s stimulation based on the efficacy of stimulation and/or the degree of relief.

39-40. (canceled)

41. The method of claim 38, wherein the method further comprises adjusting a second stimulation parameter and/or readjusting the adjusted stimulation parameter.

42-44. (canceled)

45. The method of claim 38, wherein assessing the efficacy of stimulation and/or the degree of relief of the at least one symptom comprises determining a paired-burst response ratio corresponding to quantification of the first response to the first burst-patterned stimulation as compared to quantification of the second response of the second burst-patterned stimulation.

46. The method of claim 38, wherein the method selects a first treatment for at least one symptom of a first gastrointestinal dysmotility disorder, and wherein the method is repeated to select at least a second treatment for at least one symptom of a second gastrointestinal disorder.

47. A system for treating gastrointestinal dysmotility disorder in a subject in need thereof, the system comprising:
a pulse generator that comprises a processor;
a lead electrically coupled to the device; and
an electrode electrically coupled to the lead and positioned to transmit an electrical stimulation signal to a target nerve or set of target nerves in the subject;
wherein the processor is configured to control the pulse generator to provide the electrical stimulation signal to the target nerve or the set of target nerves in the subject in a first temporal pattern comprising burst-patterned stimulation; and
wherein the application of the first temporal pattern modulates gastrointestinal motility in the subject, thereby treating the gastrointestinal dysmotility disorder.

48. The system of claim 47, wherein the pulse generator is implantable, and wherein the gastrointestinal motility disorder comprises constipation.

49-54. (canceled)

55. The system of claim 47, further comprising a programmer in communication with the pulse generator and configured to control the processor to modify one or more stimulation parameters of the electrical stimulation signal.

56. The system of claim 47, wherein the first temporal pattern comprises an interburst interval between bursts during which the burst-patterned stimulation is provided, and wherein the interburst interval is not dependent on the length of a refractory period of the subject's colon.

57. The system of claim 56, wherein the interburst interval is from about 10 seconds to about 120 seconds.

58. (canceled)

59. The system of claim 56, wherein pulse repetition frequency during each burst duration is from about 1 Hz to about 30 Hz.

60-61. (canceled)

62. The system of claim 56, wherein each burst duration is from about 20 seconds to about 60 seconds.

63. (canceled)
